(19) United States
(12) Patent Application Publication
(10) Pub. No.: US 2025/0387400 A1
(43) Pub. Date: Dec. 25, 2025

(54) TYK2 INHIBITORS

(71) Applicant: BIOGEN MA INC., Cambridge, MA (US)

(72) Inventors: Tamara Halkina Levin, Belmont, MA (US); Nathan Edward Genung, Charlestown, MA (US)

(21) Appl. No.: 18/877,312

(22) PCT Filed: Jun. 30, 2023
(86) PCT No.: PCT/US2023/026670

§ 371 Date: Dec. 20, 2024

Related U.S. Application Data
(60) Provisional application No. 63/357,762, filed on Jul. 1, 2022.

(51) Int. Cl. A61K 31/53; A61K 31/4995; A61K 31/5025; A61K 31/519; C07D 519/00

(52) U.S. Cl. CPC A61K 31/53; A61K 31/4995; A61K 31/5025; A61K 31/519; C07D 519/00

(57) ABSTRACT

This disclosure relates to compounds of Formula (I), or pharmaceutically acceptable salt thereof: Formula (I) in which all of the variables are as defined in the application. The compounds of the present disclosure are capable of inhibiting the activity of tyrosine kinase 2 (TYK2). The disclosure further provides methods of preparing the compounds of the disclosure, and methods for their therapeutic use.

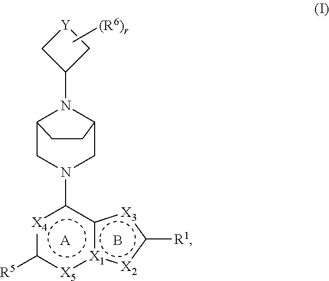

### RELATED APPLICATIONS

This application claims priority to U.S. Provisional Application No. 63/357,762 filed on Jul. 1, 2022. The entire contents of the foregoing application are expressly incorporated herein by reference.

### BACKGROUND

Cytokines are small secreted proteins released by cells and have a specific effect on the interactions and communications between cells. Cytokine pathways mediate a broad range of biological functions including many aspects of inflammation and immunity through mostly extracellular signaling.

Tyrosine kinase 2 (TYK2) is a member of Janus kinases (JAK) that are cytoplasmic protein kinases associated with cytokine receptors and play a central role in mediating cytokine signaling (Kisseleva et al., Gene, 2002, 285, 1; and Yamaoka et al. Genome Biology 2004, 5, 253). The JAK family also includes JAK1, JAK2 and JAK3. More specifically, cytokine's engagement with cognate receptors triggers activation of receptors associate with JAK, which leads to JAK mediated tyrosine phosphorylation of signal transducer and activator of transcription (STAT) proteins and ultimately transcriptional activation of specific gene sets (Schindler et al, 2007, J. Biol. Chem. 282: 20059-63). Numerous cytokines known to activate the JAK family include the interferon (IFN) family (IFN-alpha, IFN-beta, IFN-omega, Limitin, IFN-gamma, IL-10, IL-19, IL-20, IL-22), the glycoprotein (gp) 130 family (IL-6, IL-11, OSM, LlF, CNTF, NNT-1/BSF-3, G-CSF, CT-1, Leptin, IL-12, IL-23), the gamma C family (IL-2, IL-7, TSLP, IL-9, IL-15, IL-21, IL-4, IL-13), IL-3 family (IL-3, IL-5, GM-CSF), the single chain family (EPO, GH, PRL, TPO), receptor tyrosine kinases (EGF, PDGF, CSF-1, HGF), and G-protein coupled receptors (AT1).

TYK2 is important in the signaling of the type I interferons (e.g., IFN-alpha), IL-6, IL-10, IL-12 and IL-23 (Liang, Y. et al., Expert Opinion on Therapeutic Targets, 2014, 18, 5, 571-580; Kisseleva et al., 2002, Gene 285:1-24; and Watford, W. T. & O'Shea, J. J., 2006, Immunity 25:695-697). Consistent with this, primary cells derived from a TYK2 deficient human are defective in type I interferon, IL-6, IL-10, IL-12 and IL-23 signaling. TYK2 signals with other members of the JAK family in the following combinations: TYK2/JAK1, TYK2/JAK2, TYK2/JAK1/JAK2.

Studies have shown that inappropriate JAK activities can arise from mutation, over-expression, or inappropriate regulation, dys-regulation or de-regulation, as well as over- or under-production of growth factors or cytokines, and therefore trigger a variety of biological cellular responses relating to cell growth, cell differentiation, cell function, survival, apoptosis, and cell mobility. The inappropriate JAK activities are implicated in many diseases that include but not limited to cancer, cardiovascular diseases, allergies, asthma and other respiratory diseases, autoimmune diseases, inflammatory diseases, bone diseases, metabolic disorders, and neurological and neurodegenerative disorders such as Alzheimer's disease.

Small molecule JAK inhibitors have emerged as a major therapeutic advancement in treating autoimmune diseases. To date, all known small molecule JAK inhibitors that have progressed into development are active site-directed inhibitors that bind to the adenosine triphosphate (ATP) site of the catalytic domain (also referred to as the JH1 or “Janus Homology 1” domain) of the JAK protein, which prevents catalytic activity of the kinase by blocking ATP, downstream phosphorylation, and resulting pathway signal transduction (Bryan et al., J. Med. Chem. 2018, 61, 9030-9058).

Because of the high homology of the ATP active site across the kinome and especially within the JAK family, it is a significant challenge to achieve high selectivity for a specific JAK family member while also maintaining selectivity within the kinome. As a result, many JAK inhibitors that have been developed are pan-JAK inhibitors or are modestly selective for one or more JAK family members. While these inhibitors have shown encouraging results in treating autoimmune diseases, undesirable side effects leading to a narrow therapeutic index have been observed and suggest the need for improved treatments.

TYK2 has been shown to be important in the differentiation and function of multiple cell types important in inflammatory disease and autoimmune disease including natural killer cells, B cells, and T helper cell types. Aberrant TYK2 expression is associated with multiple autoimmune or inflammatory conditions.

There remains a need for potent compounds that demonstrate high selectivity for TYK2 over other members of the JAK family.

### SUMMARY

One aspect of the present disclosure is a compound of formula (I):

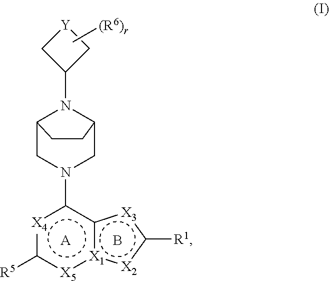

or a pharmaceutically acceptable salt thereof, wherein:
ring A and ring B together form a bicyclic heteroaryl ring;
X₁ is N or C;
X₂ is N, NH or CR²;
X₃ is N or CR³;
X₄ is N or CR⁴;
X₅ is N or CH;
Y is CR⁷R⁸, O or NR⁹;
R¹ and R⁵ are each independently selected from H, halo, CN, —NR^{1a}R^{1b}, —OR^{1c}, C₁₋₆ alkyl, C₃₋₈ cycloalkyl, C₆₋₁₀ aryl, 4 to 10 membered heterocycloalkyl and 5 to 10 membered heteroaryl, wherein the C₁₋₆ alkyl, C₃₋₈ cycloalkyl, C₆₋₁₀ aryl, 4 to 10 membered heterocycloalkyl and 5 to 10 membered heteroaryl represented by R¹ and R⁵ are each optionally substituted with one or more R¹⁰;
R², R³, and R⁴ are each independently selected from H, halo, —CN, —NR^{1a}R^{1b}, —OR^{1c}, C₁₋₄ alkyl and C₁₋₄ haloalkyl;
R⁶, for each occurrence, is independently selected from H, halo, CN, —NR^{1a}R^{1b}, —OR^{c}, —SO₂R^{1c} or C₁₋₆ alkyl optionally substituted with one or more substituents independently selected from halo, CN, —NR^{1a}R^{1b}, and —OR^{1c}; or two R⁶ together with the carbon atom from which they are attached form a 3 to 6 membered heterocycloalkyl or a C₃₋₆ cycloalkyl;
R⁷ and R⁸ are each independently selected from H, halo, CN, —NR^{1a}R^{1b}, —OR^{1c}, —SO₂R^{1c} or C₁₋₆ alkyl optionally substituted with one or more substituents independently selected from halo, CN, —NR^{1a}R^{1b}, and —OR^{1c}, or R⁷ and R⁸ together with the carbon atom from which they are attached form a 3 to 6 membered heterocycloalkyl or a C₃₋₆ cycloalkyl;
R⁹ is H or C₁₋₆alkyl optionally substituted with one or more substituents independently selected from halo, CN, —NR^{1a}R^{1b}, and —OR^{1c};
R¹⁰, for each occurrence, is independently selected from halo, —CN, —NR^{1a}R^{1b}, —OR^{1c}, —C(O)OR^{1c}, C₁₋₆ alkyl, C₁₋₆ haloalkyl, C₃₋₆ cycloalkyl, C₆₋₁₀ aryl, 4 to 7 membered monocyclic heterocycloalkyl, or 5 to 6 membered heteroaryl; wherein the C₁₋₆ alkyl, C₁₋₆ haloalkyl, C₃₋₆ cycloalkyl, C₆₋₁₀ aryl, 4 to 7 membered monocyclic heterocycloalkyl, and 5 to 6 membered heteroaryl represented by R¹⁰ are each optionally substituted with one or more substituents independently selected from halo, C₁₋₄ alkyl, C₁₋₄haloalkyl, C₃₋₆ cycloalkyl, —NR^{1a}R^{1b}, —OR^{1c} and 4 to 6 membered monocyclic heterocycloalkyl;
R^{1a} and R^{1b} are each independently H or C₁₋₄ alkyl;
R^{1c} is H, C₁₋₄ alkyl or C₁₋₄ haloalkyl; and
r is 0 or an integer from 1 to 4.

In one aspect, the present disclosure is a pharmaceutical composition comprising at least one compound described herein, or a pharmaceutically acceptable salt thereof, and at least one pharmaceutically acceptable carrier.

Another aspect of the present disclosure is a method of inhibiting TYK2 activity in a subject in need thereof comprising administering to the subject an effective amount of at least one compound described herein, or a pharmaceutically acceptable salt thereof, or a pharmaceutical composition described herein.

In some aspect, the present disclosure is a method of treating a disease or disorder responsive to inhibition of TYK2 in a subject comprising administering to the subject an effective amount of at least one compound described herein, or a pharmaceutically acceptable salt thereof, or a pharmaceutical composition described herein.

The present disclosure also includes the use of at least one compound described herein, or a pharmaceutically acceptable salt thereof, or a pharmaceutically composition described herein for the manufacture of a medicament for inhibiting TYK2 activity. Also included is the use of at least one compound described herein, or a pharmaceutically acceptable salt thereof, or a pharmaceutically composition described herein for the manufacture of a medicament for treating a disease or disorder responsive to inhibition of TYK2.

The disclosure also provides a compound described herein, or a pharmaceutically acceptable salt thereof, or a pharmaceutical composition described herein for use in inhibiting TYK2 activity. Also provided is a compound described herein, or a pharmaceutically acceptable salt thereof, or a pharmaceutical composition described herein for use in treating a disease or disorder responsive to inhibition of TYK2.

Other features or advantages will be apparent from the following detailed description of several embodiments, and also from the appended claims.

### DETAILED DESCRIPTION

The compounds or pharmaceutically acceptable salts thereof described herein demonstrate high potency against TYK2. In addition, the compounds or pharmaceutically acceptable salts thereof of the present disclosure have high selectivity for inhibiting TYK2 over other members of JAK family, such as JAK1, JAK2 and JAK3.

### I. Definitions

As used herein, the phrase “optionally substituted” is used interchangeably with the phrase “substituted or unsubstituted.” In general, the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. In some embodiments, an optionally substituted group can be substituted with one or more substituents, each of which can the same or different. In some embodiments, the “one or more” substituents can be 1, 2, 3, 4, 5, 6, etc. substituents, each of which can the same or different. In some embodiment, the “one or more” substituents can be 1 to 6, 1 to 4, 1 to 3 or 1 to 2 substituents, each of which can the same or different.

As used herein, “halogen” or “halo” may be fluorine, chlorine, bromine or iodine.

As used herein, “hydroxyl” or “hydroxy” refers to the group —OH.

As used herein, the number of carbon atoms in a group is specified herein by the prefix “Cₓ₋ₓₓ”, wherein x and xx are integers. For example, “C₁₋₄ alkyl” is an alkyl group which has from 1 to 4 carbon atoms.

As used herein, the term “alkyl” refers to a fully saturated branched or unbranched hydrocarbon moiety. Alkyl groups with 1-6 carbons, i.e., C₁₋₆ alkyl, can be preferred.

Representative examples of “alkyl” include, but are not limited to, methyl, ethyl, n-propyl, iso-propyl, n-butyl, sec-butyl, iso-butyl, tert-butyl, n-pentyl, isopentyl and neopentyl. In some embodiments, an alkyl group is a C₁₋₄ alkyl. In some embodiments, an alkyl group is a C₁₋₃ alkyl

As used herein, the term “alkenyl” refers to an unsaturated hydrocarbon group which may be linear or branched and has at least one carbon-carbon double bond. Alkenyl groups with 2-6 carbon atoms can be preferred. The alkenyl group may contain 1, 2 or 3 carbon-carbon double bonds, or more. Examples of alkenyl groups include ethenyl, n-propenyl, iso-propenyl, n-but-2-enyl, n-hex-3-enyl and the like.

As used herein, the term “haloalkyl” refers to an alkyl group as defined herein, wherein at least one of the hydrogen atoms is replaced by a halo atom. Haloalkyl groups with 1-6 carbons, i.e., C₁₋₆ haloalkyl, can be preferred. C₁₋₆ haloalkyl can be C₁₋₆ monohaloalkyl, C₁₋₆ dihaloalkyl or C₁₋₆ polyhaloalkyl including C₁₋₆ perhaloalkyl. A C₁₋₆ monohaloalkyl can have one iodo, bromo, chloro or fluoro within the alkyl group. C₁₋₆ dihaloalkyl and C₁₋₆ polyhaloalkyl groups can have two or more of the same halo atoms or a combination of different halo groups within the alkyl. Typically, the C₁₋₆ polyhaloalkyl group contains 2 to 14 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A C₁₋₆ perhaloalkyl group refers to a C₁₋₆ alkyl group having all hydrogen atoms replaced with halo atoms.

As used herein, the term “oxo” (═O) refers to an oxygen atom connected to a carbon or sulfur atom by a double bond. Examples include carbonyl, sulfinyl, or sulfonyl groups (—C(O)—, —S(O)— or —S(O)₂—) such as, a ketone, aldehyde, or part of an acid, ester, amide, lactone, or lactam group and the like.

As used herein, the terms “aryl”, “aryl group”, “aryl ring”, “aromatic group” and “aromatic ring” are used interchangeably to refer to an aromatic 6 to 12 membered monocyclic or bicyclic carbon ring system. Examples of aryl systems include, but are not limited to, phenyl, naphthyl and the like. Aryl groups with 6 to 10 membered ring system, i.e., C₆₋₁₀ aryl, can be preferred.

As used herein, the terms “heteroaryl”, “heteroaryl group”, “heteroaromatic” and “heteroaromatic ring” are used interchangeably to refer to an aromatic 5 to 12 membered monocyclic or bicyclic ring system, having at least one heteroatom (e.g., oxygen, sulfur, nitrogen, or combinations thereof), and wherein N can be oxidized (e.g., N(O)) or quaternized, and S can be optionally oxidized to sulfoxide and sulfone. Heretoaryl groups with 5 to 10 membered ring system can be preferred. “Heteroaryl” includes a heteroaromatic group that is fused to a phenyl group or non-aromatic heterocycle such as tetrahydrofuran, pyran, pyrrolidine, piperidine, and the like. Examples of heteroaryls include pyrrole, pyridyl, pyrazole, thienyl, furanyl, oxazolyl, imidazole, oxazole, isoxazole, thiazole, isothiazole, triazole, tetrazolyl, triazinyl, pyrimidyl, pyrazinyl, thiazolyl, indolyl, indazolyl, benzofuranyl, quinoxalinyl and the like. In some embodiments, heteroaryl is selected from pyrazole, imidazole, oxazole, isoxazole, thiazole, isothiazole, triazole and pyrrole.

As used herein, the term “cycloalkyl” refers to completely saturated monocyclic or bicyclic (e.g., fused, spiro or bridged) hydrocarbon groups of 3-12 carbon atoms, 3-6 carbon atoms or 5-7 carbon atoms. Cycloalkyl groups with 3-8 carbons, i.e., C₃₋₈ cycloalkyl, can be preferred. Examples of C₃₋₈ cycloalkyl include, but are not limited to, cyclopropyl, cyclobutyl, cyclopentyl, and cyclohexyl.

As used herein, the term “heterocycloalkyl” refers to completely saturated 4 to 12 membered monocyclic or bicyclic (e.g., fused) ring system, having at least one heteroatom (e.g., oxygen, sulfur, nitrogen, or combinations thereof). In some embodiments, the heterocycloalkyl group has 1, 2, 3 or 4 heteroatoms (preferably, 1 or 2 heteroatoms). In some embodiments, Heterocycloalkyl groups with 4 to 10 membered ring system can be preferred.

In some embodiments, the heterocycloalkyl group has 4 to 6 ring atoms (i.e., 4 to 6 membered heterocycloalkyl) with 1 or 2 heteroatoms independently selected from oxygen and nitrogen). In some embodiments, the heterocycloalkyl group has 4 to 6 ring atoms with 1 heteroatom that is oxygen. In some embodiments, the heterocycloalkyl group is oxetane.

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds described herein, for example, compound of formula (I), (II), (III), (IV), (V), (VI), (VII), (IIA), (IIB), (IIIA), (IIIB), (IVA) or (IVB), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts. The compounds of the present disclosure, may inherently or by design form salts, hydrates and solvates, polymorphs thereof.

As used herein, the term “a,” “an,” “the” and similar terms used in the context of the present disclosure (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the disclosure and does not pose a limitation on the scope of the disclosure otherwise claimed.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

It will be recognized by those skilled in the art that the compounds of the present disclosure may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

Certain of the compounds described herein contain one or more asymmetric centers or axes and may thus give rise to enantiomers, diastereomers, and other stereoisomeric forms that may be defined, in terms of absolute stereochemistry, as (R)- or (S)-. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase). In some embodiment, the compounds described herein are isolated stereoisomers wherein each of the compounds has one stereocenter and the stereoisomer is in the R configuration. In other embodiment, the compounds described herein are isolated stereoisomers wherein each of the compounds has one stereocenter and the stereoisomer is in the S configuration. In one embodiment, the compounds described herein are isolated stereoisomers wherein each of the compounds has two stereocenters and the stereoisomer is in the R R configuration. In one embodiment, the compounds described herein are isolated stereoisomers wherein each of the compounds has two stereocenters and the stereoisomer is in the R S configuration. In one embodiment, the compounds described herein are isolated stereoisomers stereoisomer wherein each of the compounds has two stereocenters and the stereoisomer is in the S R configuration. In one embodiment, the compounds described herein are isolated stereoisomers stereoisomer wherein each of the compounds has two stereocenters and the stereoisomer is in the S S configuration. In one embodiment, the compounds described herein each have one or two stereocenters and are racemic mixtures.

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a particular enantiomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired enantiomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers is at least 20%, 30%, 40%, 50%, 60%, 70%, 80%, 90%, 95%, 97%, 99%, 99.5% or 99.9%. The stereoisomeric purity means the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or IPLC chromatography columns, such as CHIRALPAK™ and CHIRALCEL™ available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated. All such forms are embraced within the scope of the disclosure. In addition, some compounds may exhibit polymorphism. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

Furthermore, the compounds of the present disclosure, including their salts, may also be obtained in the form of their hydrates, or include other solvents used for their crystallization. The compounds of the present disclosure may inherently or by design form solvates with pharmaceutically acceptable solvents (including water); therefore, it is intended that the disclosure embrace both solvated and unsolvated forms. The term “solvate” refers to a molecular complex of a compound of the present disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Compounds of the disclosure that contain groups capable of acting as donors and/or acceptors for hydrogen bonds may be capable of forming co-crystals with suitable co-crystal formers. These co-crystals may be prepared from the compounds by known co-crystal forming procedures. Such procedures include grinding, heating, co-subliming, co-melting, or contacting in solution the compounds with the co-crystal former under crystallization conditions and isolating co-crystals thereby formed. Suitable co-crystal formers include those described in WO 2004/078163. Hence the disclosure further provides co-crystals comprising a compound described herein.

In one embodiment, the disclosure provides deuterated compounds disclosed herein, in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance.

### II. Compounds of the Disclosure

In a first embodiment, the compound of the disclosure is represented by Formula (I), or a pharmaceutically acceptable salt thereof, wherein the variables are as described above.

In some embodiments, X₅ is N.

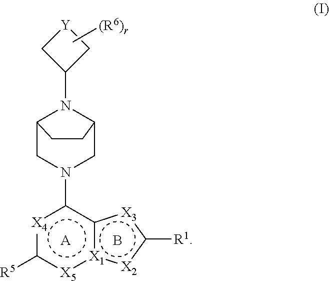

In a second embodiment, for compounds of formula (I) or pharmaceutically acceptable salts thereof, X₂ is NH or CR²; and the remaining variables are as described in the first embodiment. In some embodiments, X₂ is NH or CH.

In a third embodiment, the compound is represented by Formula (II), (III), (IV), (V), (VI) or (VII):

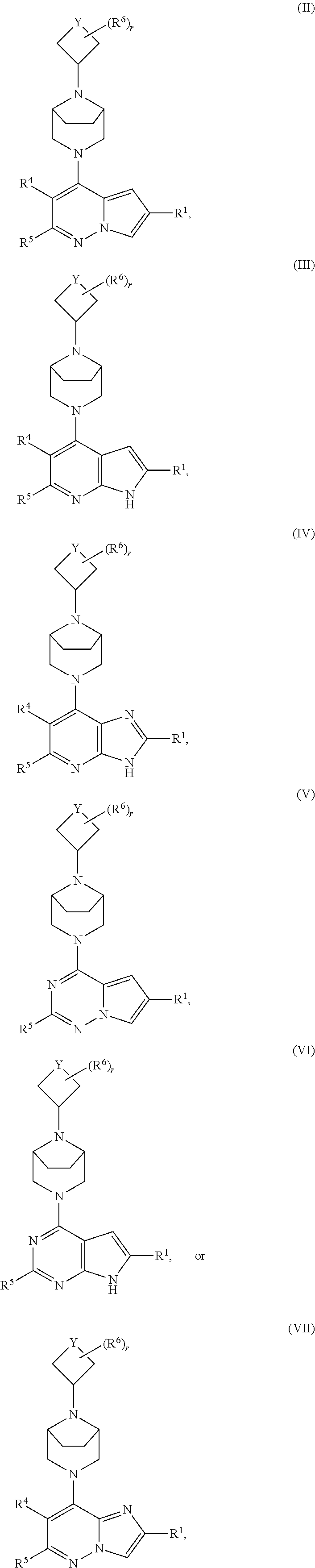

or a pharmaceutically acceptable salt thereof, and the variables in Formulae (II), (III), (IV), (V), (VI) and (VII) are as described in the first embodiment.

In a fourth embodiment, for compounds of formula (I), (II), (III), (IV), (V), (VI) or (VII), or pharmaceutically acceptable salts thereof, Y is CR⁷R⁸; and the remaining variables are as described in the first, second or third embodiment.

In a fifth embodiment, for compounds of formula (I), (II), (III), (IV), (V), (VI) or (VII), or pharmaceutically acceptable salts thereof, Y is O; and the remaining variables are as described in the first, second or third embodiment.

In a sixth embodiment, for compounds of formula (I), (II), (III), (IV), (V), (VI) or (VII), or pharmaceutically acceptable salts thereof, Y is NR⁹; and the remaining variables are as described in the first, second or third embodiment.

In a seventh embodiment, for compounds of formula (I), (II), (III), (IV), (V), (VI) or (VII), or pharmaceutically acceptable salts thereof, R¹ is a 5 to 6-membered heteroaryl optionally substituted with 1, 2 or 3 R¹⁰; and the remaining variables are as described in the first, second, third, fourth, fifth or sixth embodiment.

In an eighth embodiment, for compounds of formula (I), (II), (III), (IV), (V), (VI) or (VII), or pharmaceutically acceptable salts thereof, R¹ is a 5 to 6-membered heteroaryl selected from pyrazole, imidazole, oxazole, isoxazole, thiazole, isothiazole, triazole, pyrrole, pyridine, pyridazine, pyrimidine and pyrazine, each of which is optionally substituted with 1, 2 or 3 R¹⁰; and the remaining variables are as described in the first, second, third, fourth, fifth, sixth or seventh embodiment.

In a ninth embodiment, for compounds of formula (I), (II), (III), (IV), (V), (VI) or (VII), or pharmaceutically acceptable salts thereof, R¹ is a 5 to 6-membered heteroaryl selected from pyrazole, isoxazole, pyridine and pyridazine, each of which is optionally substituted with 1 or 2 R¹⁰; and the remaining variables are as described in the first, second, third, fourth, fifth, sixth or seventh embodiment.

In a tenth embodiment, for compounds of formula (I), (II), (III), (IV), (V), (VI) or (VII), or pharmaceutically acceptable salts thereof, R¹ is a 5 to 6-membered heteroaryl selected from:

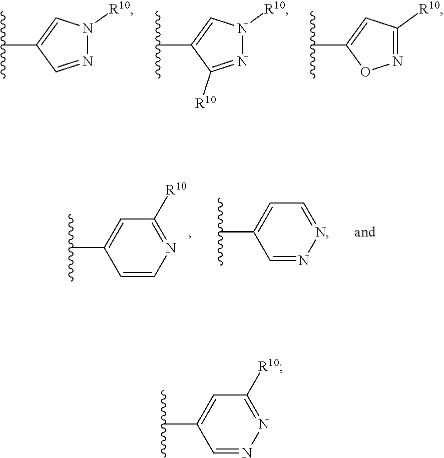

wherein

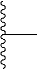

represents a point of attachment to the ring B; and the remaining variables are as described in the first, second, third, fourth, fifth, sixth, seventh, eighth or ninth embodiment.

In an eleventh embodiment, for compounds of formula (I), (II), (III), (IV), (V), (VI) or (VII), or pharmaceutically acceptable salts thereof, R¹⁰ is independently selected from halo, —OR^{1c}, C₁₋₆ alkyl and C₁₋₆ haloalkyl; and R^{1c} is C₁₋₄ alkyl; and the remaining variables are as described in the first, second, third, fourth, fifth, sixth, seventh, eighth, ninth or tenth embodiment.

In a twelfth embodiment, for compounds of formula (I), (II), (III), (IV), (V), (VI) or (VII), or pharmaceutically acceptable salts thereof, R¹⁰ is independently selected from F, —OCH₃, —CH₃ and —CHF₂; and the remaining variables are as described in the first, second, third, fourth, fifth, sixth, seventh, eighth, ninth, tenth or eleventh embodiment.

In a thirteenth embodiment, for compounds of formula (I), (II), (III), (IV), (V), (VI) or (VII), or pharmaceutically acceptable salts thereof, R², R³, and R⁴ are each independently selected from H, halo, C₁₋₄ alkyl and C₁₋₄ haloalkyl; and the remaining variables are as described in the first, second, third, fourth, fifth, sixth, seventh, eighth, ninth, tenth, eleventh or twelfth embodiment.

In a fourteenth embodiment, for compounds of formula (I), (II), (III), (IV), (V), (VI) or (VII), or pharmaceutically acceptable salts thereof, R², R³, and R⁴ are H; and the remaining variables are as described in the first, second, third, fourth, fifth, sixth, seventh, eighth, ninth, tenth, eleventh, twelfth or thirteenth embodiment.

In a fifteenth embodiment, for compounds of formula (I), (II), (III), (IV), (V), (VI) or (VII), or pharmaceutically acceptable salts thereof, R⁵ is H, halo, C₁₋₄ alkyl, C₁₋₄haloalkyl or 5 to 6-membered heteroaryl optionally substituted with 1, 2 or 3 R¹⁰; and the remaining variables are as described in the first, second, third, fourth, fifth, sixth, seventh, eighth, ninth, tenth, eleventh, twelfth, thirteenth or fourteenth embodiment.

In a sixteenth embodiment, for compounds of formula (I), (II), (III), (IV), (V), (VI) or (VII), or pharmaceutically acceptable salts thereof, R⁵ is H; and the remaining variables are as described in the first, second, third, fourth, fifth, sixth, seventh, eighth, ninth, tenth, eleventh, twelfth, thirteenth, fourteenth or fifteenth embodiment.

In a seventeenth embodiment, for compounds of formula (I), (II), (III), (IV), (V), (VI) or (VII), or pharmaceutically acceptable salts thereof, R⁷ and R⁸ are each independently selected from H, halo, CN, —OR^{1c}, —SO₂R^{1c}, and C₁₋₆ alkyl optionally substituted with one or more substituents independently selected from halo, CN, and —OR^{1c}, or R⁷ and R⁸ together with the carbon atom from which they are attached form a 4-6 membered heterocycloalkyl; and R^{1c} is H or C₁₋₃alkyl; and the remaining variables are as described in the first, second, third, fourth, fifth, sixth, seventh, eighth, ninth, tenth, eleventh, twelfth, thirteenth, fourteenth, fifteenth or sixteenth embodiment.

In an eighteenth embodiment, for compounds of formula (I), (II), (III), (IV), (V), (VI) or (VII), or pharmaceutically acceptable salts thereof, R⁷ and R⁸ are each independently selected from H, F, CN, —OH, —OCH₃, —SO₂CH₃, —CH₃, —CHF₂, —CF₃, —CH₂—CN, —CH₂—O—CH₃, and —CH₂CH₂—O—CH₃, or R⁷ and R⁸ together with the carbon atom from which they are attached form:

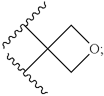 [SMILES: *C1(*)COC1]

and the remaining variables are as described in the first, second, third, fourth, fifth, sixth, seventh, eighth, ninth, tenth, eleventh, twelfth, thirteenth, fourteenth, fifteenth, sixteenth or seventeenth embodiment.

In a nineteenth embodiment, for compounds of formula (I), (II), (III), (IV), (V), (VI) or (VII), or pharmaceutically acceptable salts thereof, r is 0, 1 or 2; and the remaining variables are as described in the first, second, third, fourth, fifth, sixth, seventh, eighth, ninth, tenth, eleventh, twelfth, thirteenth, fourteenth, fifteenth, sixteenth, seventeenth or eighteenth embodiment.

In a twentieth embodiment, for compounds of formula (I), (II), (III), (IV), (V), (VI) or (VII), or pharmaceutically acceptable salts thereof, R⁶, for each occurrence, is independently selected from H, —OR^{1c}, C₁₋₄haloalkyl and C₁₋₄ alkyl optionally substituted with CN, or two R⁶ together with the carbon atoms from which they are attached form a 4-6 membered heterocycloalkyl; and R^{1c} is H or C₁₋₃alkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth or nineteenth embodiment.

In a twenty-first embodiment, for compounds of formula (I), (II), (III), (IV), (V), (VI) or (VII), or pharmaceutically acceptable salts thereof, R⁶, for each occurrence, is independently selected from H, —OH, —OCH₃, —CH₂—CN, and —CH₃, or two R⁶ together with the carbon atom from which they are attached form:

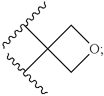 [SMILES: *C1(*)COC1]

and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth or twentieth embodiment.

In a twenty-second embodiment, for compounds of formula (I), (II), (III), (IV), (V), (VI) or (VII), or pharmaceutically acceptable salts thereof, R¹ is a 5 to 6-membered heteroaryl optionally substituted with 1, 2 or 3 R¹⁰; R², R³, and R⁴ are each independently selected from H, halo, C₁₋₄ alkyl and C₁₋₄ haloalkyl; R⁵ is H, halo, C₁₋₄ alkyl, C₁₋₄ haloalkyl or 5 to 6-membered heteroaryl optionally substituted with 1, 2 or 3 R¹⁰; R⁶, for each occurrence, is independently selected from H, —OR^{1c}, C₁₋₄haloalkyl and C₁₋₄alkyl optionally substituted with CN, or two R⁶ together with the carbon atoms from which they are attached form a 4 membered heterocycloalkyl; R⁷ and R⁸ are each independently selected from H, halo, CN, —OR^{1c}, —SO₂R^{1c}, and C₁₋₆ alkyl optionally substituted with one or more substituents independently selected from halo, CN, and —OR^{1c}, or R⁷ and R⁸ together with the carbon atom from which they are attached form a 4 membered heterocycloalkyl; R⁹ is H or C₁₋₆ alkyl; R¹⁰ is independently selected from halo, —OR^{1c}, C₁₋₆ alkyl and C₁₋₆ haloalkyl; R^{1a} and R^{1b} are each independently H or C₁₋₄ alkyl; R^{1c} is H, C₁₋₄ alkyl; and r is 0, 1 or 2; and the remaining variables are as described in the first, second, third, fourth, fifth or sixth embodiment.

In a twenty-third embodiment, for compounds of formula (I), (II), (III), (IV), (V), (VI) or (VII), or pharmaceutically acceptable salts thereof, R¹ is a 5 to 6-membered heteroaryl selected from pyrazole, imidazole, oxazole, isoxazole, thiazole, isothiazole, triazole, pyrrole, pyridine, pyridazine, pyrimidine and pyrazine, each of which is optionally substituted with 1, 2 or 3 R¹⁰; R², R³, and R⁴ are H; R⁵ is H, halo, C₁₋₄ alkyl or C₁₋₄ haloalkyl; R⁶, for each occurrence, is independently selected from H, —OR^{1c}, C₁₋₄haloalkyl and C₁₋₄ alkyl optionally substituted with CN, or two R⁶ together with the carbon atoms from which they are attached form a 4-6 membered heterocycloalkyl; R⁷ and R⁸ are each independently selected from H, halo, CN, —OR^{1c}, —SO₂R^{1c}, and C₁₋₆ alkyl optionally substituted with one or more substituents independently selected from halo, CN, and —OR^{1c}, or R⁷ and R⁸ together with the carbon atom from which they are attached form a 4-6 membered heterocycloalkyl; R⁹ is H or C₁₋₄ alkyl; R¹⁰ is independently selected from halo, —OR^{1c}, C₁₋₄alkyl and C₁₋₄haloalkyl; and R^{1c} is H or C₁₋₄alkyl; and the remaining variables are as described in the first, second, third, fourth, fifth or sixth embodiment.

In a twenty-fourth embodiment, the compound is represented by Formula (IIA), (IIB), (IIIA), (IIIB), (IVA) or (IVB):

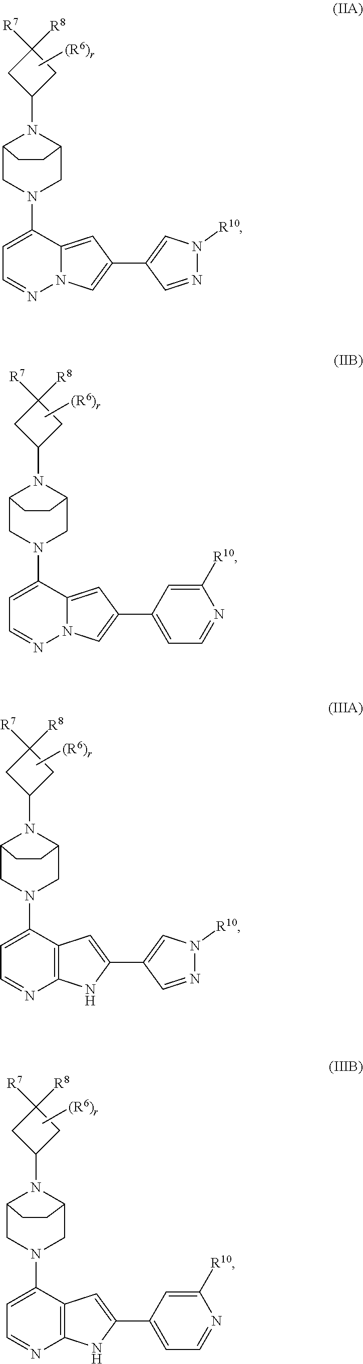

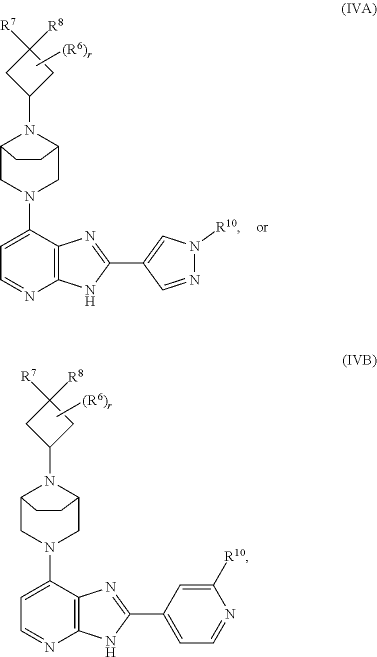

or a pharmaceutically acceptable salt thereof, and the variables of Formulae (IIA), (IIB), (IIIA), (IIIB), (IVA) are (IVB) are as described in the first, eleventh, twelfth, seventeenth, eighteenth, nineteenth, twentieth, twenty-first, twenty-second or twenty-third embodiment.

In a twenty-fifth embodiment, for compounds of formula (I), (II), (IIA), (IIB), (III), (IIIA), (IIIB), (IV), (IVA), (IVB), (V), (VI) or (VII), or pharmaceutically acceptable salts thereof, R⁶, for each occurrence, is independently selected from H and C₁₋₄alkyl; R⁷ and R⁸ are each independently selected from H, halo, CN, and C₁₋₄alkyl; R¹⁰ is selected from —OR^{1c}, C₁₋₄alkyl, and C₁₋₄haloalkyl; R^{1c} is C₁₋₄alkyl; and r is 0, 1 or 2; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third or twenty-fourth embodiment.

In a twenty-sixth embodiment, for compounds of formula (I), (II), (IIA), (IIB), (III), (IIIA), (IIIB), (IV), (IVA), (IVB), (V), (VI) or (VII), or pharmaceutically acceptable salts thereof, R⁶ is H or CH₃; R⁷ and R⁸ are each independently selected from H, F, CH₃ and CN; R¹⁰ is selected from CH₃, CHF₂ and OCH₃; and r is 0, 1 or 2; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third or twenty-fourth embodiment.

In a twenty-seventh embodiment, for compounds of formula (I), (II), (IIA), (IIB), (III), (IIIA), (IIIB), (IV), (IVA), (IVB), (V), (VI) or (VII), or pharmaceutically acceptable salts thereof, r is 0; and the remaining variables are as described in the twenty-fourth, twenty-fifth or twenty-sixth embodiment.

In a twenty-eighth embodiment, the compound selected from any one of of the following
(1R,3r)-1-methyl-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile; (1S,3s)-1-methyl-3-(3-(2-(1-methyl-1H-pyrazol-4-yl)-3H-imidazo[4,5-b]pyridin-7-yl)-3,8-diazabicyclo[3.2.1]octan-8-yl)cyclo butane-1-carbonitrile; (1R,3r)-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile; (1S,3s)-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile; 7-(8-((1r,3R)-3-methoxycyclobutyl)-3,8-diazabicyclo[3.2.1]octan-3-yl)-2-(1-methyl-1H-pyrazol-4-yl)-3H-imidazo[4,5-b]pyridine; 7-(8-((1s,3S)-3-methoxycyclobutyl)-3,8-diazabicyclo[3.2.1]octan-3-yl)-2-(1-methyl-1H-pyrazol-4-yl)-3H-imidazo[4,5-b]pyridine; (1RS,2RS)-2-(3-(2-(1-methyl-1H-pyrazol-4-yl)-3H-imidazo[4,5-b]pyridin-7-yl)-3,8-diaza bicyclo[3.2.1]octan-8-yl)cyclobutan-1-ol; (1RS,2SR)-2-(3-(2-(1-methyl-1H-pyrazol-4-yl)-3H-imidazo[4,5-b]pyridin-7-yl)-3,8-diazabicyclo[3.2.1]octan-8-yl)cyclobutan-1-ol; 2-(1-methyl-1H-pyrazol-4-yl)-7-(8-(oxetan-3-yl)-3,8-diazabicyclo[3.2.1]octan-3-yl)-3H-imidazo[4,5-b]pyridine; 7-((1R,5S)-8-(3-(difluoromethyl)cyclobutyl)-3,8-diazabicyclo[3.2.1]octan-3-yl)-2-(1-methyl-1H-pyrazol-4-yl)-3H-imidazo[4,5-b]pyridine; 7-(8-(azetidin-3-yl)-3,8-diazabicyclo[3.2.1]octan-3-yl)-2-(1-methyl-1H-pyrazol-4-yl)-3H-imidazo[4,5-b]pyridine hydrochloride; 2-(1-methyl-1H-pyrazol-4-yl)-7-(8-(1-methylazetidin-3-yl)-3,8-diazabicyclo[3.2.1]octan-3-yl)-3H-imidazo[4,5-b]pyridine trifluoroacetate; 3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile; (1S,3s)-3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile;
(1R,3r)-3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile; (1S,3s)-3-(3-(2-(1-methyl-1H-pyrazol-4-yl)-1H-pyrrolo[2,3-b]pyridin-4-yl)-3,8-diazabicyclo[3.2.1]octan-8-yl)cyclobutane-1-carbonitrile; (1S,3s)-3-(3-(2-(1-methyl-1H-pyrazol-4-yl)-1H-pyrrolo[2,3-b]pyridin-4-yl)-3,8-diazabicyclo[3.2.1]octan-8-yl)cyclobutane-1-carbonitrile; 2-(2-methoxypyridin-4-yl)-4-(8-(oxetan-3-yl)-3,8-diazabicyclo[3.2.1]octan-3-yl)-1H-pyrrolo[2,3-b]pyridine; 4-(8-(3-(difluoromethyl)cyclobutyl)-3,8-diazabicyclo[3.2.1]octan-3-yl)-2-(2-methoxypyridin-4-yl)-1H-pyrrolo[2,3-b]pyridine; 2-[3-[3-[6-(1-methylpyrazol-4-yl)pyrrolo[1,2-b]pyridazin-4-yl]-3,8-diazabicyclo[3.2.1]octan-8-yl]oxetan-3-yl]acetonitrile; 3-[3-[6-(1-methylpyrazol-4-yl)pyrrolo[1,2-b]pyridazin-4-yl]-3,8-diazabicyclo[3.2.1]octan-8-yl]cyclobutane-1-carbonitrile; 3-[3-[6-(1-methylpyrazol-4-yl)pyrrolo[1,2-b]pyridazin-4-yl]-3,8-diazabicyclo[3.2.1]octan-8-yl]-1-(trifluoromethyl) cyclobutan-1-ol; 6-(1-methylpyrazol-4-yl)-4-[8-(oxetan-3-yl)-3,8-diazabicyclo[3.2.1]octan-3-yl]pyrrolo[1,2-b]pyridazine; 4-[8-(3-methoxycyclobutyl)-3,8-diazabicyclo[3.2.1]octan-3-yl]-6-(1-methylpyrazol-4-yl)pyrrolo[1,2-b]pyridazine; 3-[3-[6-(1-methylpyrazol-4-yl)pyrrolo[1,2-b]pyridazin-4-yl]-3,8-diazabicyclo[3.2.1]octan-8-yl]cyclobutan-1-ol; 4-(8-cyclobutyl-3,8-diazabicyclo[3.2.1]octan-3-yl)-6-(1-methylpyrazol-4-yl)pyrrolo[1,2-b]pyridazine; (1R,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; (1S,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; 4-(8-((1r,3R)-3-methoxycyclobutyl)-3,8-diazabicyclo[3.2.1]octan-3-yl)-6-(1-methyl-1H-pyrazol-4-yl) pyrrolo[1,2-b]pyridazine; 4-(8-((1s,3S)-3-methoxycyclobutyl)-3,8-diazabicyclo[3.2.1]octan-3-yl)-6-(1-methyl-1H-pyrazol-4-yl)pyrrolo[1,2-b]pyridazine; (1R,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol; (1S,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol; 6-(1-methylpyrazol-4-yl)-4-[8-(2-oxaspiro[3.3]heptan-6-yl)-3,8-diazabicyclo[3.2.1]octan-3-yl]pyrrolo[1,2-b]pyridazine; (1r,3r)-1-methyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; (1s,3s)-1-methyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; 6-(1-methyl-1H-pyrazol-4-yl)-4-(8-((1s,3s)-3-(methylsulfonyl)cyclobutyl)-3,8-diazabicyclo[3.2.1]octan-3-yl)pyrrolo[1,2-b]pyridazine; 6-(1-methyl-1H-pyrazol-4-yl)-4-(8-((1r,3r)-3-(methylsulfonyl)cyclobutyl)-3,8-diazabicyclo[3.2.1]octan-3-yl)pyrrolo[1,2-b]pyridazine; (1r,3r)-1-(2-methoxyethyl)-3-(3-(6-(1-methyl-1H-pyrazol-4-yl)pyrrolo[1,2-b]pyridazin-4-yl)-3,8-diazabicyclo[3.2.1]octan-8-yl) cyclobutane-1-carbonitrile; (1s,3s)-1-(2-methoxyethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; 4-(8-((S)-2-oxaspiro[3.3]heptan-5-yl)-3,8-diazabicyclo[3.2.1]octan-3-yl)-6-(1-methyl-1H-pyrazol-4-yl)pyrrolo[1,2-b]pyridazine; 4-(8-((R)-2-oxaspiro[3.3]heptan-5-yl)-3,8-diazabicyclo[3.2.1]octan-3-yl)-6-(1-methyl-1H-pyrazol-4-yl)pyrrolo[1,2-b]pyridazine; (1s,3s)-1-(methoxymethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; (1r,3r)-1-(methoxymethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; 2-((1s,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutyl)acetonitrile; 2-((1r,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutyl)acetonitrile; Rac-(1R,3R)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; (1R,3S)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; (1S,3R)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile; rac-(1S,2S)-2-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol; rac-(1R,2S)-2-(3-(6-(1-methyl-1H-pyrazol-4-yl)pyrrolo[1,2-b]pyridazin-4-yl)-3,8-diaza bicyclo[3.2.1]octan-8-yl)cyclobutan-1-ol; Rac-4-(8-((1S,2S)-2-methoxycyclobutyl)-3,8-diaza bicyclo[3.2.1]octan-3-yl)-6-(1-methyl-1H-pyrazol-4-yl)pyrrolo[1,2-b]pyridazine; 4-[8-(3,3-difluorocyclobutyl)-3,8-diazabicyclo[3.2.1]octan-3-yl]-6-(1-methylpyrazol-4-yl)pyrrolo[1,2-b]pyridazine; 6-(6-methoxypyridazin-4-yl)-4-[8-(oxetan-3-yl)-3,8-diazabicyclo[3.2.1]octan-3-yl]pyrrolo[1,2-b]pyridazine; 3-[3-[6-(6-methoxypyridazin-4-yl)pyrrolo[1,2-b]pyridazin-4-yl]-3,8-diazabicyclo[3.2.1]octan-8-yl]cyclobutane-1-carbonitrile; 3-[3-(6-pyridazin-4-ylpyrrolo[1,2-b]pyridazin-4-yl)-3,8-diazabicyclo[3.2.1]octan-8-yl]cyclobutane-1-carbonitrile; 4-[8-(oxetan-3-yl)-3,8-diazabicyclo[3.2.1]octan-3-yl]-6-pyridazin-4-ylpyrrolo[1,2-b]pyridazine; 3-[3-[6-(6-methylpyridazin-4-yl)pyrrolo[1,2-b]pyridazin-4-yl]-3,8-diazabicyclo[3.2.1]octan-8-yl]cyclobutane-1-carbonitrile; (1R,3r)-3-(3-(6-(3-methylisoxazol-5-yl)pyrrolo[1,2-b]pyridazin-4-yl)-3,8-diazabicyclo[3.2.1]octan-8-yl)cyclobutane-1-carbonitrile; (1S,3s)-3-(3-(6-(3-methyl isoxazol-5-yl)pyrrolo[1,2-b]pyridazin-4-yl)-3,8-diazabicyclo[3.2.1]octan-8-yl)cyclobutane-1-carbonitrile; (1S,3s)-3-(3-(6-(1-(difluoromethyl)-1H-pyrazol-4-yl)pyrrolo[1,2-b]pyridazin-4-yl)-3,8-diazabicyclo[3.2.1]octan-8-yl)cyclobutane-1-carbonitrile; (1S,3s)-3-(3-(6-(1-methyl-1H-pyrazol-4-yl)pyrrolo[2,1-f][1,2,4]triazin-4-yl)-3,8-diazabicyclo[3.2.1]octan-8-yl)cyclobutane-1-carbonitrile; (1R,3r)-3-(3-(6-(1-methyl-1H-pyrazol-4-yl)pyrrolo[2,1-f][1,2,4]triazin-4-yl)-3,8-diazabicyclo[3.2.1]octan-8-yl)cyclobutane-1-carbonitrile; (1R,3r)-3-(3-(6-(1-methyl-1H-pyrazol-4-yl)-7H-pyrrolo[2,3-d]pyrimidin-4-yl)-3,8-diazabicyclo[3.2.1]octan-8-yl)cyclobutane-1-carbonitrile; (1S,3s)-3-(3-(6-(1-methyl-1H-pyrazol-4-yl)-7H-pyrrolo[2,3-d]pyrimidin-4-yl)-3,8-diazabicyclo[3.2.1]octan-8-yl)cyclobutane-1-carbonitrile; (1S,3s)-3-(3-(2-(1-methyl-1H-pyrazol-4-yl)imidazo[1,2-b]pyridazin-8-yl)-3,8-diazabicyclo[3.2.1]octan-8-yl)cyclobutane-1-carbonitrile; and (1R,3r)-3-(3-(2-(1-methyl-1H-pyrazol-4-yl)imidazo[1,2-b]pyridazin-8-yl)-3,8-diaza bicyclo[3.2.1]octan-8-yl)cyclobutane-1-carbonitrile, or a pharmaceutically acceptable salt thereof.

### III. Pharmaceutical Compositions

In another embodiment, the present disclosure is a pharmaceutical composition comprising at least on compound described herein, or a pharmaceutically acceptable salt thereof, and at least one pharmaceutically acceptable carrier.

As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The formulations may be prepared using conventional dissolution and mixing procedures. For example, the bulk drug substance (i.e., compound of the present disclosure or stabilized form of the compound (e.g., complex with a cyclodextrin derivative or other known complexation agent)) is dissolved in a suitable solvent in the presence of one or more of the excipients described above. The compound of the present disclosure is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

### IV. Use of Compounds and Compositions of the Disclosure

The compounds, or pharmaceutically acceptable salts thereof described herein may be used to decrease or inhibit the activity of TYK2 or to otherwise affect the properties and/or behavior of TYK2, e.g., stability, phosphorylation, kinase activity, interactions with other proteins, etc.

In some embodiment, the present disclosure provides a method of inhibiting TYK2 activity in a subject in need thereof comprising administering to the subject an effective amount of at least one compound described herein, or a pharmaceutically acceptable salt thereof, or a pharmaceutical composition described herein.

As used herein, the term “inhibit”, “inhibition” or “inhibiting” refers to the reduction or suppression or a significant decrease in the baseline activity of a biological activity or process.

One embodiment of the present disclosure is a method of treating a disease or disorder responsive to inhibition of TYK2 in a subject comprising administering to the subject an effective amount of at least one compound described herein, or a pharmaceutically acceptable salt thereof, or a pharmaceutical composition described herein. In some embodiment, the method described herein treats the disease or disorder responsive to inhibition of TYK2, wherein the disease or disorder includes inflammation, autoimmune disease, neuroinflammation, arthritis, rheumatoid arthritis, spondyloarthropathies, systemic lupus erythematous, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis and sunburn.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis.

The term “inflammatory disorders” includes diseases or disorders involving acute or chronic inflammation such as allergies, asthma, atopic dermatitis, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer).

As used herein, the term “subject” and “patient” may be used interchangeably, and means a mammal in need of treatment, e.g., human, companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 g-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The a compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F.

Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)). The protection of functional groups by protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present disclosure having at least one salt-forming group may be prepared in a manner known to those skilled in the art. For example, acid addition salts of compounds of the present disclosure are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a commercially available chiral HPLC column.

The disclosure further includes any variant of the present processes, in which the reaction components are used in the form of their salts or optically pure material. Compounds of the disclosure and intermediates can also be converted into each other according to methods generally known to those skilled in the art.

For illustrative purposes, the reaction described below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### EXEMPLIFICATIONS

The compounds of the Examples described below were analyzed or purified according to one of the Purification Methods referred to below unless otherwise described.

### Abbreviations

Aq. means aqueous;
Bn means benzyl;
Boc means tert-butoxy carbonyl;
Boc₂O means di-tert-butyl dicarbonate
br means broad;
t-BuOH means tertiary butanol
n-BuLi means n-butyl lithium;
° C. means degrees Celsius;
CDCl₃ means deutero-chloroform;
δ means chemical shift;
d means doublet;
dd means double doublet;
DCM means dichloromethane;
DEA means diethylamine
DIPEA means N-ethyldiisopropylamine or N,N-diisopropylethylamine;
DMA means N,N-dimethylacetamide;
DMF means N,N-dimethylformamide;
DMSO means Dimethylsulfoxide;
DMSO-d₆ means hexadeuterodimethyl sulfoxide;
Et means ethyl;
EtOH means ethanol;
EtOAc means ethyl acetate;
Eq. means equivalent;
g means gram;
HCl means hydrochloric acid;
HCO₂H means formic acid
¹H NMR means proton nuclear magnetic resonance;
H₂O means water;
HPLC means high pressure liquid chromatography;
h means hour;
IPA means 2-propanol
K₂CO₃ means potassium carbonate;
KF means potassium fluoride
KOH means potassium hydroxide;
L means litre;
LCMS means liquid chromatography mass spectrometry;
LDA means lithium diisopropylamide;
m means multiplet;
M means molar;
Me means methyl;
MeCN means acetonitrile;
Mel means iodomethane
MeOH means methanol;
MeOH-d₄ means deutero-methanol;
mg means milligram;
MgSO₄ means magnesium sulfate;
MHz means mega Hertz;
mins means minutes;
mL means millilitres;
mmol means millimole;
MS m/z means mass spectrum peak;
MsCl means methanesulfonyl chloride;
N₂ means nitrogen;
NaBH₄ means sodium borohydride;
NaBH₃CN means sodium cycanoborohydride;
Na₂CO₃ means sodium carbonate;
NaH means sodium hydride;
NaHCO₃ means sodium bicarbonate;
NaIO₄ means sodium periodate;
NaOH means sodium hydroxide;
Na₂SO₄ means sodium sulfate;
NBS means N-bromosuccinimide;
NH₃ means ammonia;
NH₄Cl means ammonium chloride;
NH₄OH is ammonium hydroxide;
NOE means nuclear Overhauser effect spectroscopy;
PE means petroleum ether;
Pd(amphos)Cl₂ means bis(di-tert-butyl(4-dimethylaminophenyl)phosphine)dichloropalladium(II)
Pd₂(dba)₃ means tris(dibenzylideneacetone)dipalladium (0);
Pd(dppf)Cl₂ means [1,1′-bis(diphenylphosphino)ferrocene]dichloropalladium(II);
PdCl₂(PPh₃)₂ means bis(triphenylphosphine)palladium(II) dichloride;
Pd/C means palladium on charcoal;
POCl₃ means phosphorous oxychloride;
q means quartet;
rt means room temperature;
RuCl₃ means ruthenium (III) chloride;
s means singlet;
sat. means saturated;
SFC means supercritical fluid chromatography;
soln. means solution;
t means triplet;
TBME means tert-butyl methyl ether;
TEA means triethylamine;
TFA means trifluoroacetic acid;
THF means tetrahydrofuran;
TLC means thin layer chromatography;
TsCl means para-toluenesulfonyl chloride;
TsOH means para-toluenesulfonic acid
μL means micro litres;
μmol means micromole; and
RuPhos Pd G3 means (2-dicyclohexylphosphino-2′,6′-diisopropoxy-1,1′-biphenyl)[2-(2′-amino-1,1′-biphenyl)]palladium(II) methanesulfonate.

### General Synthetic Methods

According to a first process, compounds of Formula (I) may be prepared from compounds of Formulae (AII), (AIII), (AIV) and (AV) as illustrated in Scheme 1.

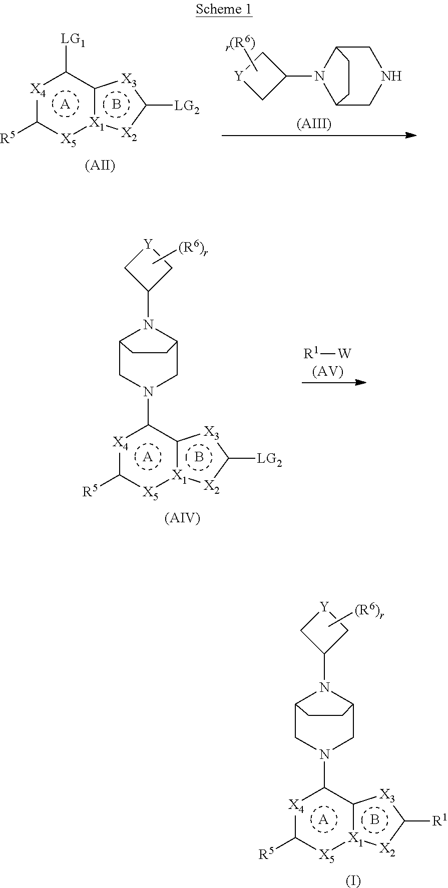

LG₁ and LG₂ are a suitable leaving groups, typically a halogen such as Cl, Br or I or a sulfonate such as mesylate, tosylate or triflate.

W is a boronic acid or suitable boronate ester such as boronic acid pinacol ester.

Compounds of Formula (AIV) may be prepared from the compound of Formula (AII) and the compound of the Formula (AIII) using a suitable nucleophilic substitution reaction such as an S_{N}Ar under thermal or transition metal catalysed conditions. Typical conditions comprise, reaction of the amine of Formula (AIII) with the compound of Formula (AII) in the presence of a suitable inorganic base, a suitable palladium catalyst in the presence of suitable phosphine ligands, in a suitable solvent at elevated temperature, optionally under microwave irradiation. Preferred conditions comprise, reaction of the compounds of Formulae (AII) and (AIII) in the presence of RuPhos PdG3 or Xantphos, optionally in combination with Pd₂(dba)₃, in the presence of a suitable base such as K₂CO₃, K₃PO₄ or Cs₂CO₃ in a suitable solvent such as dioxane, THE or toluene at between 70° C. and 110° C., optionally under microwave irradiation. Alternatively, the compounds of Formulae (AII) and (AIII) may be reacted together in the presence of a suitable base, such as TEA, in an appropriate aprotic solvent at between 70° C. and 110° C., optionally under microwave irradiation.

The compound of Formula (I) may be prepared from the compounds of Formulae (AIV) and (AV) by a palladium catalysed, cross-coupling reaction, such as a Suzuki reaction. Typical cross-coupling reaction conditions comprise a palladium catalyst containing suitable phosphine ligands, in the presence of an inorganic base, in a suitable solvent at between rt and the reflux temperature of the reaction, optionally in the presence of microwave irradiation. Preferred conditions comprise, reaction of the compound of Formula (AIV) and (AV) in the presence of Pd(dppf)Cl₂ or Pd(amphos)Cl₂, and a suitable base such as Na₂CO₃, K₂CO₃ or KF in a suitable solvent such as dioxane, optionally in the presence of water, at between 70° C. and 100° C.

According to a second process, compounds of Formula (I) may be prepared from compounds of Formulae (AII), (AVI), (AV), (AVII), (AVIII) and (AIX) as illustrated in Scheme 2.

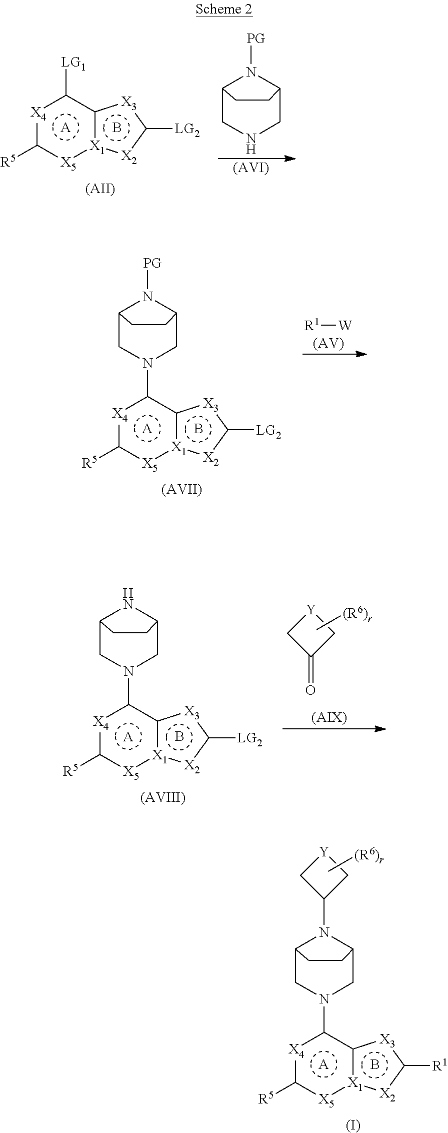

LG₁ and LG₂ are a suitable leaving groups, typically a halogen such as Cl, Br or I or a sulfonate such as mesylate, tosylate or triflate.

W is a boronic acid or suitable boronate ester such as boronic acid pinacol ester.

PG is a typical amine protecting group such as carbamate and preferably Boc.

Compounds of Formula (AVII) may be prepared from the compound of Formula (AII) and the compound of the Formula (AVI) using a suitable nucleophilic substitution reaction such as an S_{N}Ar under thermal or transition metal catalysed conditions. Typical conditions comprise, reaction of the amine of Formula (AVI) with the compound of Formula (AII) in the presence of a suitable inorganic base, a suitable palladium catalyst in the presence of suitable phosphine ligands, in a suitable solvent at elevated temperature, optionally under microwave irradiation. Preferred conditions comprise, reaction of the compounds of Formulae (AII) and (AVI) in the presence of RuPhos PdG3 or Xantphos, optionally in combination with Pd₂(dba)₃, in the presence of a suitable base such as K₂CO₃, K₃PO₄ or Cs₂CO₃ in a suitable solvent such as dioxane, THE or toluene at between 70° C. and 110° C., optionally under microwave irradiation. Alternatively, the compounds of Formulae (AII) and (AVI) may be reacted together in the presence of a suitable base, such as TEA, in an appropriate aprotic solvent at between 70° C. and 110° C., optionally under microwave irradiation.

The compound of Formula (AVIII) may be prepared from the compounds of Formulae (AV) and (AVII) by a palladium catalysed, cross-coupling reaction, such as a Suzuki reaction. Typical cross-coupling reaction conditions comprise a palladium catalyst containing suitable phosphine ligands, in the presence of an inorganic base, in a suitable solvent at between rt and the reflux temperature of the reaction, optionally in the presence of microwave irradiation. Preferred conditions comprise, reaction of the compound of Formula (AV) and (AVII), in the presence of Pd(dppf)Cl₂ or Pd(amphos)Cl₂, and a suitable base such as Na₂CO₃, K₂CO₃ or KF in a suitable solvent such as dioxane, optionally in the presence of water, at between 70° C. and 100° C. A subsequent amine deprotection step affords compounds of Formula (AVIII) preferably via a typical removal of Boc reaction under acidic conditions such as HCl or TFA in an aprotic solvent such as DCM at 0° C. to rt.

Compounds of Formula (I) may be prepared from the compounds of Formulae (AVIII) and (AIX) using a reductive amination process in the presence of a suitable reducing agent in a suitable solvent at a temperature between 0° C. and elevated temperature. Preferred conditions involve reacting an amine of Formula (AVIII) with a ketone of Formula (AIX) in the presence of an appropriate reducing agent, such as NaBH₃CN, in a suitable protic solvent such as MeOH at a temperature from rt to reflux temperature, preferably at 50° C.

According to a third process, compounds of Formula (I) may be prepared from compounds of Formulae (AIV), (AX) and (AXI) as illustrated in Scheme 3.

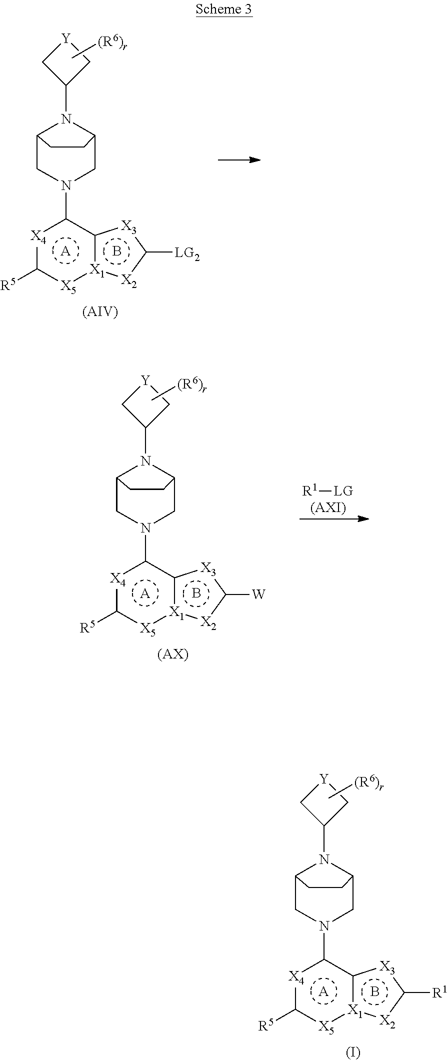

LG₁ and LG₂ are a suitable leaving groups, typically a halogen such as Cl, Br or I or a sulfonate such as mesylate, tosylate or triflate.

W is a boronic acid or suitable boronate ester such as boronic acid pinacol ester.

The compound of Formula (AIV) may be transformed into a compound of Formula (AX) using a boronate ester formation reaction achieved by treatment with a suitable boronate such as (BPin)₂, in the presence of a suitable inorganic base, such as K₂CO₃ or KOAc and a suitable catalyst, such as, Pd(dppf)Cl₂ or Pd₂(dba)₃ in a suitable non-polar solvent, such as MeCN at between rt and elevated temperature.

The compound of Formula (I) may be prepared from compounds of Formulae (AX) and (AXI) using a palladium catalysed, cross-coupling reaction, such as a Suzuki reaction. Typical cross-coupling reaction conditions comprise a palladium catalyst containing suitable phosphine ligands, in the presence of an inorganic base, in a suitable solvent at between rt and the reflux temperature of the reaction, optionally in the presence of microwave irradiation. Preferred conditions comprise, reaction of the compound of Formula (AX) and (AXI), in the presence of Pd(dppf)Cl₂ or Pd(amphos)Cl₂, and a suitable base such as Na₂CO₃, K₂CO₃ or KF in a suitable solvent such as dioxane, optionally in the presence of water, at between 70° C. and 100° C.

According to a fourth process, compounds of Formula (I) may be prepared from compounds of Formulae (AVII), (AXII), (AXI), (AVIII) and (AIX) as illustrated in Scheme 4.

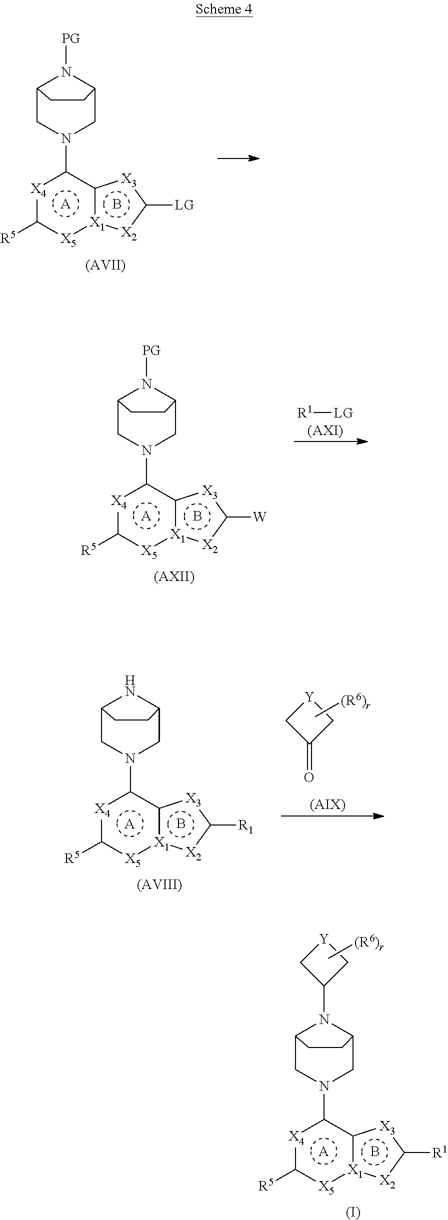

LG₁ and LG₂ are a suitable leaving groups, typically a halogen such as Cl, Br or I or a sulfonate such as mesylate, tosylate or triflate.

W is a boronic acid or suitable boronate ester such as boronic acid pinacol ester.

The compound of Formula (AVII) may be transformed into a compound of Formula (AXII) using a boronate ester formation reaction achieved by treatment with a suitable boronate such as (BPin)₂, in the presence of a suitable inorganic base, such as K₂CO₃ or KOAc and a suitable catalyst, such as, Pd(dppf)Cl₂ or Pd₂(dba)₃ in a suitable non-polar solvent, such as MeCN at between rt and elevated temperature.

The compound of Formula (AVIII) may be prepared from compounds of Formulae (AXI) and (AXII) using a palladium catalysed, cross-coupling reaction, such as a Suzuki reaction. Typical cross-coupling reaction conditions comprise a palladium catalyst containing suitable phosphine ligands, in the presence of an inorganic base, in a suitable solvent at between rt and the reflux temperature of the reaction, optionally in the presence of microwave irradiation. Preferred conditions comprise, reaction of the compound of Formula (AXI) and (AXII), in the presence of Pd(dppf)Cl₂ or Pd(amphos)Cl₂, and a suitable base such as Na₂CO₃, K₂CO₃ or KF in a suitable solvent such as dioxane, optionally in the presence of water, at between 70° C. and 100° C. A subsequent amine deprotection step affords compounds of Formula (AVIII) preferably via a typical removal of Boc reaction under acidic conditions such as HCl or TFA in an aprotic solvent such as DCM at 0° C. to rt.

Compounds of Formula (I) may be prepared from the compounds of Formulae (AVIII) and (AIX) using a reductive amination process in the presence of a suitable reducing agent in a suitable solvent at a temperature between 0° C. and elevated temperature. Preferred conditions involve reacting an amine of Formula (AVIII) with a ketone of Formula (AIX) in the presence of an appropriate reducing agent, such as NaBH₃CN, in a suitable protic solvent such as MeOH at a temperature from rt to reflux temperature, preferably at 50° C.

According to a fifth process, wherein, X₂ and X₃ are N and X₁ is C, compounds of Formula (I), may be prepared from compounds of Formulae (AIII), (AXIII), (AXIV), (AXV) and (AXVI) as illustrated in Scheme 5.

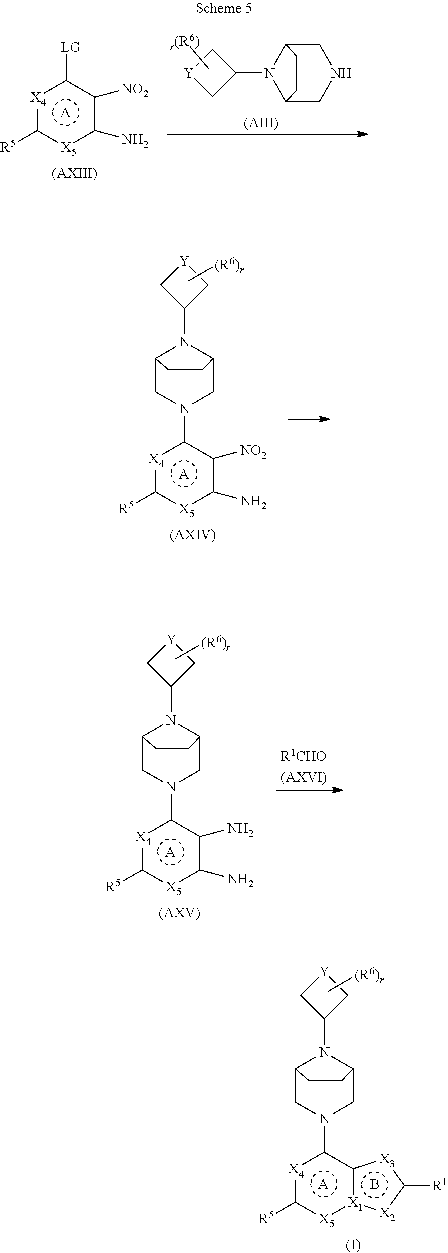

LG is a suitable leaving group, typically a halogen such as Cl, Br or I or a sulfonate such as mesylate, tosylate or triflate.

The compound of Formula (AXIV) may be prepared by reaction of the compounds of Formulae (AIII) and (AXIII), in the presence of a suitable base and a suitable aprotic polar solvent at between 0° C. and elevated temperature. Preferred conditions, comprise reaction of the compound of Formula (AIII) with the compound of Formula (AXIII) in the presence of TEA in DMSO or DMF at ambient temperature to 90° C.

The compound of Formula (AXV) may be prepared from the compound of Formula (AXIV) by a reduction reaction, typically in the presence of a suitable hydrogenation catalyst, such as Pd/C in a suitable alcoholic solvent such as MeOH under an atmosphere of H₂ at room temperature.

The compound of Formula (I) may be prepared from the di-amine of Formula (AXV) and the aldehyde of Formula (AXVI) via a condensation reaction in the presence of a suitable acidic catalyst, such as TsOH in a suitable polar aprotic solvent, such as DMF at elevated temperature, such as between 50° C. and 80° C.

According to a sixth process, compounds of Formula (I)(B) wherein one occurrence of R⁶ is CH₂EWG may be prepared from a compound of Formulae (AXIII) (or AVIII) and (AYY) as illustrated in Scheme 6.

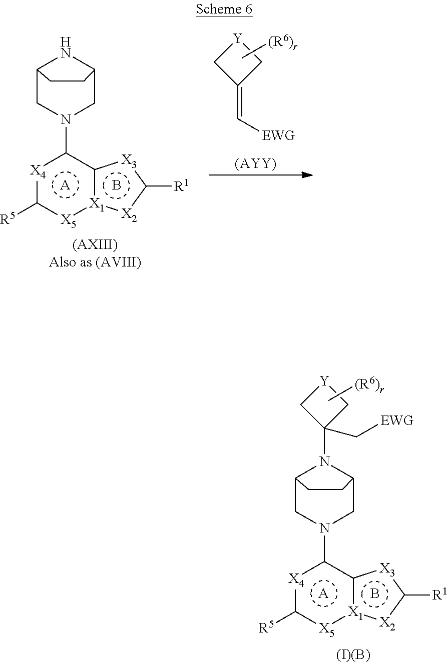

EWG is an electron withdrawing group as defined within R⁶.

The compound of Formula (I)(B) may be prepared by reaction of the compounds of Formulae (AXIII) (or AVIII) and (AYY), in the presence of a suitable base and a suitable aprotic polar solvent at a suitable temperature optionally in a sealed tube. Preferred conditions, comprise reaction of the compound of Formula (AXIII) (or AVIII) with the compound of Formula (AYY) in the presence of TEA in DMSO or DMF at 90° C. in a sealed vessel.

According to a seventh process, compounds of Formula (AIII) may be prepared from compounds of Formulae (AXVIII), (AXIX), (AXX), (AXXI) and (AXXII) as illustrated in Scheme 7.

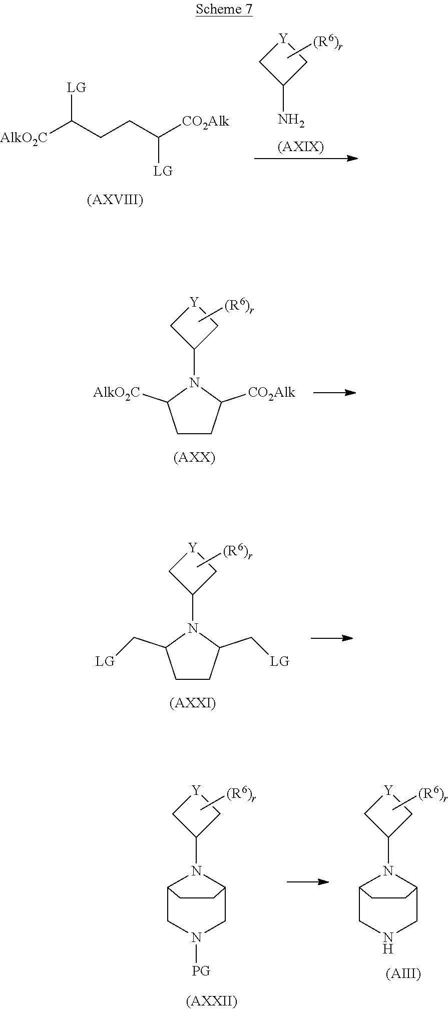

LG is a suitable leaving group.

The compound of Formula (AXX) may be prepared by reaction of an amine of Formula (AXIX) with a compound of Formula (AXVIII) in the presence of a suitable base in an aprotic solvent at ambient to elevated temperature. Preferred conditions, comprise reaction of the compound of Formula (AXVIII) with the compound of Formula (AXIX) in the presence of K₂CO₃ in MeCN under reflux. Compound of the Formula (AXXI) may be obtained after function group interconversion of the compound of Formula (AXX) using methods selected a skilled person in the art. In particular, a compound of Formula (AXX) may be prepared using a sequential exhaustive hydride reduction using an appropriate reducing agent such as LiAlH₄ in THF. Subsequent conversion of the intermediate alcohols into appropriate leaving groups, LG, to afford compound of the Formula (AXXI). Compound of the Formula (AXXII), where PG is benzyl, may be prepared from a compound of Formula (AXXI) and benzylamine amine in the presence of a suitable base, such as K₂CO₃, in an aprotic solvent, such as MeCN, at ambient to reflux temperature. Subsequent hydrogenolysis of the benzyl group (where PG is benzyl) in the presence of a suitable catalyst, such as Pd/C in a suitable protic solvent such as MeOH under H₂ in an autoclave at 50° C. affords a compound of Formula (AIII).

Compounds of Formula (I) or (AIV) may be converted to alternative compounds of Formula (I) or (AIV), by standard chemical transformations such as for example, alkylation of a heteroatom such as N, via reductive amination, or halogenation, such as fluorination, using methods well known to those skilled in the art.

The compounds of Formulae (AII), (AIII), (AV), (AVI), (AVII), (AIX), (AXI), (AXIV) and (AXVII) are commercially available, may be prepared by analogy to methods known in the literature, or the methods described in the Experimental section below.

It will be appreciated by those skilled in the art that it may be necessary to utilise a suitable protecting group strategy for the preparation of compounds of Formula (I). Typical protecting groups may comprise, carbamate and preferably Boc for the protection of amines, or a Tosyl group for the protection of imidazole N atoms.

It will be appreciated by those skilled in the art that it may be necessary to utilise a suitable functional group interconversion strategy for intermediates en route to the preparation of compounds of Formula (I).

Chromatography on silica gel was carried out using 20-40 μM (particle size), 250-400 mesh, or 400-632 mesh silica gel using either a Teledyne ISCO Combiflash RF or a Grace Reveleris X₂ with ELSD purification systems.

### Analytical Methods

### ESI-MS:

ESI-MS data (also reported herein as simply MS) were recorded using Waters System (Acquity HPLC and a Micromass ZQ mass spectrometer); all masses reported are the m/z of the protonated parent ions unless recorded otherwise.

### LCMS:

The sample is dissolved in a suitable solvent such as MeCN, DMSO or MeOH and is injected directly into the column using an automated sample handler. The analysis using one of the following methods:

### Analytical HPLC:

Acidic HPLC: Conducted on a Shimadza 20A instrument with an ultimate C18 3.0×50 mm, 3 μm column eluting with 2.75 mL/4 L TFA in water (solvent A) and 2.5 mL/4 L TFA in acetonitrile (solvent B).

### Analytical LCMS:

Acidic LCMS: Conducted on a Shimadza 2010 Series, Shimadza 2020 Series, or Waters Acquity UPLC BEH. (MS ionization: ESI) instrument equipped with a C18 column (2.1 mm×30 mm, 3.0 mm or 2.1 mm×50 mm, C18, 1.7 μm), eluting with 1.5 mL/4 L TFA in water (solvent A) and 0.75 mL/4 L TFA in acetonitrile (solvent B).

### SFC Analytical Separation:

Instrument: Waters UPC2 analytical SFC (SFC-H). Column: ChiralCel OJ, 150×4.6 mm I.D., 3 μm. Mobile phase: A for CO₂ and B for Ethanol (0.05% DEA). Gradient: B 40%. Flow rate: 2.5 mL/min. Back pressure: 100 bar. Column temperature: 35° C. Wavelength: 220 nm

### Preparative HPLC Purification:

The following codes refer to the preparative HPLC conditions used as indicated in the examples and preparation sections. Individual gradients were optimised for each example as appropriate.

TABLE 1
HPLC Conditions

| Prep-HPLC | |
|---|---|
| Code | Conditions |
| HPLC-1 | Phenomenex Synergi C18 150 × 30 mm, 4 μm; |
| | MeCN/0.05% HCl; gradient 0-100% optimised for |
| | each sample |
| HPLC-2 | Phenomenex Synergi C18; 150 × 30 mm, 4 μm; |
| | MeCN/0.225% HCO₂H; gradient |
| | 0-100% optimised for each example |
| HPLC-3 | Welch Xtimate C18 150 × 25 mm; 5 μm; MeCN/(10 mM |
| | NH₄HCO₃); gradient 0-100% |
| | optimised for each sample |
| HPLC-4 | Welch Xtimate C18; 150 × 30 mm, 5 μm; MeCN/H₂O + |
| | 10 mM NH₄HCO₃; gradient 0-100% |
| | optimised for each example |
| HPLC-5 | YMC-Actus Triart C18 100 × 30 mm; 5 μm, MeCN/H₂O |
| | (0.1% TFA); gradient 0-100% optimised for each sample |
| HPLC-6 | Boston Prime C18 150 × 30 mm, 5 μm; MeCN/(0.05% |
| | NH₄OH + 10 mM NH₄HCO₃); |
| | gradient 0-100% optimised for each sample |

### Preparative SFC Purification

Instrument: MG III preparative SFC (SFC-1). Column, mobile phase and gradient are shown in Table 3. Flow rate: 40 mL/min. Back pressure: 100 bar. Column temperature: 38° C. Wavelength: 220 nm. Cycle time: ˜8 min.

### ¹H-NMR:

TABLE 2
SFC Purification Conditions

| Prep-SFC | |
|---|---|
| Code | Conditions |
| SFC-1 | Daicel Chiralpak AD 250 mm × 30 mm, 10 μm; ROH |
| | (+0.1% NH₄OH) in CO₂. |
| | ROH optimised for each example. |
| SFC-2 | Diacel Chiralpak AS-H, 250 × 30 mm, 10 μm; 30% |
| | IPA (+0.1% DEA) in CO₂ |
| SFC-3 | Diacel Chiralcel OJ-H, 250 × 30 mm, 5 mm; 35% EtOH |
| | (+0.1% DEA) in CO₂ |
| SFC-4 | LUX Cellulose-4 LC, 30 × 250 mm, 5 mm; 40% MeOH |
| | (+0.1% DEA) in CO₂. |
| SFC-5 | CHIRALPAK IB 30 × 250 mm, 5 mm; 35% MeOH |
| | (+0.1% DEA) in CO₂. |
| SFC-6 | Diacel Chiralpak OD-H, 250 × 30 mm, 5 mm); 60% EtOH |
| | (+0.1% NH₄OH) in CO₂ |
| SFC-7 | Diacel Chiralcel OJ-H, 250 × 30 mm, 5 mm; 35% EtOH |
| | (+0.1% NH₄OH) in CO₂ |

The NMR spectra were recorded on Bruker Avance III HD 500 MHz, Bruker Avance III 500 MHz, Bruker Avance III 400 MHz, Varian-400 VNMRS, or Varian-400 MR. Chemical shifts are expressed in parts per million (ppm) units. Coupling constants (J) are in units of hertz (Hz). Splitting patterns describe apparent multiplicities and are designated as s (single), d (double), t (triplet), dd (double doublet), dt (double triplet), dq (double quartet), m (multiplet), br (broad).

Typically, the compounds described herein can be prepared according to the schemes provided below. The following examples serve to illustrate the disclosure without limiting the scope thereof. Methods for preparing such compounds are described hereinafter.

### GENERAL PROCEDURES

### Preparation Methods

### Preparation 1

### 1-(methoxymethyl)-3-oxocyclobutane-1-carbonitrile

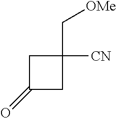

Part 1: 3-Methylenecyclobutane-1-carbonitrile (5 g, 53.7 mmol) was added dropwise to a stirred solution of LDA (60 mmol) in THE (50 mL) at −78° C. under an inert atmosphere and the resulting mixture stirred for 1 h. Chloro(methoxy)methane (5.2 g, 64.6 mmol) was added dropwise at −78° C. and the reaction mixture warmed to rt and stirred overnight. The resulting mixture was partitioned between saturated NH₄Cl solution (100 mL) and EtOAc (100 mL). The organic layer was separated, washed with brine (100 mL), dried (Na₂SO₄) and evaporated to dryness in vacuo to afford 1-(methoxymethyl)-3-methylenecyclobutane-1-carbonitrile (3.7 g) which was used without further purification.

Part 2: A single crystal of RuCl₃ was added to a stirred solution of 1-(methoxymethyl)-3-methylenecyclobutane-1-carbonitrile (Preparation 1, Part 1, 3.7 g) and NaIO₄ (23 g, 107.5 mmol) in a mixture of MeCN/CCl₄ (40 mL, 1:1) and water (30 mL) and the mixture was stirred overnight at rt. The solids were removed by filtration and the filtrate separated, washed with brine (20 mL), dried (Na₂SO₄.) and evaporated to dryness in vacuo. The residue was purified by column chromatography (EtOAc/Hex, 1:4) to give 1-(methoxymethyl)-3-oxocyclobutane-1-carbonitrile (1.2 g, 16% over 2 steps). ¹H NMR (500 MHz, DMSO-d₆) δ: 3.65 (s, 2H), 3.61-3.48 (m, 2H), 3.46 (s, 3H), 3.38-3.28 (m, 2H).

### Preparation 2

### 1-(2-methoxyethyl)-3-oxocyclobutane-1-carbonitrile

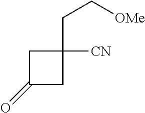 [SMILES: COCCC1(C#N)CC(=O)C1]

The title compound was prepared from 3-methylenecyclobutane-1-carbonitrile and 1-bromo-2-methoxyethane using an analogous method to that described for Preparation 1. Yield: 1.5 g, 18% over 2 steps. ¹H NMR (400 MHz, CDCl₆) δ: 3.67-3.55 (m, 4H), 3.35-3.23 (m, 5H), 2.11 (t, 2H).

### Preparation 3

### 3-methylene-1-((methylsulfonyl)methyl)cyclobutane-1-carbonitrile

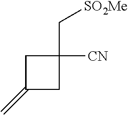

Part 1: n-BuLi (2.5 M, 200 mL, 500 mmol) was added dropwise to diisopropylamine (52.15 g, 515.4 mmol) in THF (1500 mL) at 0° C. and the resulting mixture stirred for 0.5 h at the same temperature. The reaction mixture was cooled to −78° C. and 3-methylenecyclobutane-carbonitrile (40 g, 429.5 mmol) in THE (300 mL) was added dropwise and the mixture stirred for 1 h. (Chloromethyl)(methyl)sulfane (49.8 g, 515.4 mmol) in THE (300 mL) was added dropwise and the reaction mixture stirred overnight. The reaction was quenched with aq. Sol. of NH₄Cl and the organic phase separated. The combined organics were dried (Na₂SO₄) and evaporated to dryness in vacuo. The residue was distilled (1 mBar, 50° C.) to obtain 3-methylene-1-((methylthio)methyl)cyclobutane-1-carbonitrile (12 g).

Part 2: RuCl₃ (0.1 g) was added to a solution of 3-methylene-1-((methylthio)methyl)cyclo-butenecarbonitrile (12 g, 78 mmol) in a mixture of MeCN/H₂O (400 mL) and the solution cooled to 0° C. Sodium periodate (90 g, 468 mmol) was added in portions and the resulting mixture stirred overnight at rt. The solids were removed by filtration and the filtrate evaporated to dryness in vacuo. The residue was washed with EtOAc and the solvent was evaporated under reduced pressure. The residue was recrystallized from MeCN/TPS to afford 3-methylene-1-((methylsulfonyl)methyl)cyclobutane-1-carbonitrile (5.05 g). ¹H NMR (400 MHz, DMSO-d₆) δ: 4.05 (s, 2H), 3.87-3.75 (m, 2H), 3.67-3.58 (m, 2H), 3.13 (s, 3H).

### Preparation 4

### Cis diethyl 1-(3,3-difluorocyclobutyl)pyrrolidine-2,5-dicarboxylate

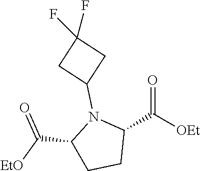 [SMILES: CCOC(=O)[C@H]1CC[C@@H](C(=O)OCC)N1C1CC(F)(F)C1]

Solid K₂CO₃ (57.7 g, 418 mmol) was added to a stirred solution of 3,3-difluorocyclobutan-1-amine hydrochloride (20 g, 139 mmol) and diethyl 2,5-dibromohexanedioate (50.2 g, 139 mmol) in MeCN (250 mL) and the resulting mixture heated under reflux for 16 h. The reaction mixture was diluted with water (500 mL) and extracted with EtOAc (250 mL). The combined organics were washed with brine (200 mL), dried (Na₂SO₄) and evaporated to dryness in vacuo give diethyl 1-(3,3-difluorocyclobutyl)pyrrolidine-2,5-dicarboxylate as a mixture of diastereomers (40 g) which were separated by flash column chromatography (hexane/MTBE gradient elution) to afford cis diethyl 1-(3,3-difluorocyclobutyl)pyrrolidine-2,5-dicarboxylate as a yellow oil (17.4 g, 40.9%).

### Preparation 5

### Cis-(1-(3,3-difluorocyclobutyl)pyrrolidine-2,5-diyl)dimethanol

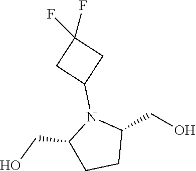 [SMILES: OC[C@@H]1CC[C@H](CO)N1C1CC(F)(F)C1]

A solution of cis diethyl 1-(3,3-difluorocyclobutyl)pyrrolidine-2,5-dicarboxylate (Preparation 4, 17.4 g, 57 mmol) in THE (40 mL) was added dropwise at 0 C to a stirred solution of LiAlH₄ (4.33 g, 114 mmol) in THE (160 mL) and the resulting mixture stirred for 16 h. The reaction was quenched by the dropwise addition of water (4.3 mL), followed by 50% aq. NaOH (4.3 g) and water (13 mL). The precipitate was removed by filtration through celite and washed with THF. The filtrate was evaporated under reduced pressure and the residue dissolved in DCM (100 mL), dried (Na₂SO₄) evaporated to afford cis-(1-(3,3-difluorocyclobutyl)pyrrolidine-2,5-diyl)dimethanol (11 g, 87.4%).

### Preparation 6

### Cis 1-(3,3-difluorocyclobutyl)pyrrolidine-2,5-diyl)bis(methylene) dimethanesulfonate

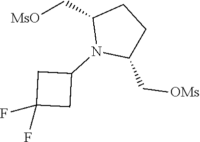 [SMILES: CS(=O)(=O)OC[C@H]1CC[C@@H](COS(C)(=O)=O)N1C1CC(F)(F)C1]

Methanesulfonyl chloride (14.25 g, 124.5 mmol) was added dropwise to a stirred solution of cis-(1-(3,3-difluorocyclobutyl)pyrrolidine-2,5-diyl)dimethanol (Preparation 5, 11 g, 49.8 mmol) and TEA (20.8 mL, 149 mmol) in DCM (150 mL) at 0° C. and stirring was continued for 1 h at this temperature. The reaction mixture was diluted with H₂O (200 mL) and DCM (100 mL). The combined organics were washed with aq. Na₂CO₃, dried (Na₂SO₄) and evaporated under reduced pressure to give cis 1-(3,3-difluorocyclobutyl)pyrrolidine-2,5-diyl)bis(methylene) dimethanesulfonate (18.3 g, 97.3%).

### Preparation 7

### 3-benzyl-8-(3,3-difluorocyclobutyl)-3,8-diazabicyclo[3.2.1]octane

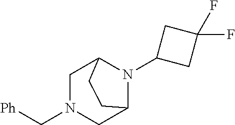 [SMILES: FC1(F)CC(N2C3CCC2CN(Cc2ccccc2)C3)C1]

Potassium carbonate (20.7 g, 150 mmol) and NaI (0.75 g, 5 mmol) were added to a stirred solution of cis 1-(3,3-difluorocyclobutyl)pyrrolidine-2,5-diyl)bis(methylene) dimethanesulfonate (Preparation 6, 18.3 g, 48.5 mmol) and benzylamine (5.24 g, 49 mmol) in MeCN (250 mL) and the mixture heated under reflux for 16 h. The reaction mixture was diluted with water (250 mL) and extracted with EtOAc (2×150 mL). The combined organics were washed with brine (150 mL), dried (Na₂SO₄) and evaporated to dryness in vacuo. The residue was purified by flash column chromatography (MTBE/MeOH; gradient elution) to afford 3-benzyl-8-(3,3-difluorocyclobutyl)-3,8-diazabicyclo[3.2.1]octane (4.8 g, 33.8%).

### Preparation 8

### 8-(3,3-difluorocyclobutyl)-3,8-diazabicyclo[3.2.1]octane dihydrochloride

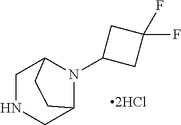 [SMILES: FC1(F)CC(N2C3CCC2CNC3)C1]

HCl in Et₂O (20% wt., 15 mL) was added to a solution of 3-benzyl-8-(3,3-difluorocyclobutyl)-3,8-diazabicyclo[3.2.1]octane (Preparation 7, 4.8 g, 16.4 mmol) in MeOH (25 mL) and the solvents were evaporated in vacuo. The residue was dissolved in MeOH (50 mL) and 10% Pd/C (1 g) was added and the resulting mixture was stirred under 50 atm of H₂ at 50° C. in autoclave for 64 h. Then the mixture was filtered through celite and the filtrate evaporated under reduced pressure and the residue was triturated with Et₂O (100 mL) to obtain 8-(3,3-difluorocyclobutyl)-3,8-diazabicyclo[3.2.1]octane dihydrochloride (2.21 g, 48.8%). ¹H NMR (500 MHz, DMSO-d₆) δ: 12.85 (s, 1H), 10.24 (s, 1H), 10.04 (s, 1H), 4.05 (s, 2H), 3.79-3.74 (m, 3H), 3.4-3.1 (m, 4H), 2.94 (s, 2H), 2.28 (s, 4H).

### Preparation 9

### tert-butyl 3-(2-amino-3-nitropyridin-4-yl)-3,8-diazabicyclo[3.2.1]octane-8-carboxylate

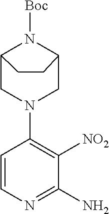 [SMILES: CC(C)(C)OC(=O)N1C2CCC1CN(c1ccnc(N)c1[N+](=O)[O-])C2]

To a solution of tert-butyl 3,8-diazabicyclo[3.2.1]octane-8-carboxylate (200.0 mg, 0.942 mmol) in DMF (5.0 mL) was added DIPEA (146.1 mg, 1.13 mmol) and 4-chloro-3-nitropyridin-2-amine (163.5 mg, 0.942 mmol) and the reaction stirred at 90° C. for 2 h. The cooled mixture was concentrated in vacuo and the residue purified by silica gel column chromatography (PE/EtOAc=2/1) to give tert-butyl 3-(2-amino-3-nitropyridin-4-yl)-3,8-diazabicyclo[3.2.1]octane-8-carboxylate (260.0 mg, 79.0% yield) as a yellow solid.

¹H NMR (400 MHz, MeOH-d₄) δ: 7.78 (d, 1H), 6.48 (d, 1H), 4.25 (s, 2H), 3.20-3.14 (m, 4H), 1.93-1.81 (m, 4H), 1.49 (s, 9H).

### Preparation 10

### tert-butyl 3-(2,3-diaminopyridin-4-yl)-3,8-diazabicyclo[3.2.1]octane-8-carboxylate

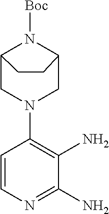 [SMILES: CC(C)(C)OC(=O)N1C2CCC1CN(c1ccnc(N)c1N)C2]

To a solution of tert-butyl 3-(2-amino-3-nitropyridin-4-yl)-3,8-diazabicyclo[3.2.1]octane-8-carboxylate (Preparation 9, 215.0 mg, 0.615 mmol) in MeOH (3.0 mL) was added Pd/C (39.3 mg, 0.037 mmol, 10% purity) under 15 psi of H₂ and the reaction stirred at 25° C. for 1 h. The reaction was filtered and the filtrate concentrated in vacuo to afford tert-butyl 3-(2,3-diaminopyridin-4-yl)-3,8-diazabicyclo[3.2.1]octane-8-carboxylate (160.0 mg, 81.4% yield) as a yellow solid.

¹H NMR (500 MHz, MeOH-d₄) δ: 7.39-7.37 (m, 1H), 6.47-6.45 (m, 1H), 4.29 (s, 2H), 3.00-2.84 (m, 4H), 2.08-1.98 (m, 4H), 1.49 (s, 9H).

### Preparation 11

### tert-butyl 3-(2-(1-methyl-1H-pyrazol-4-yl)-3H-imidazo[4,5-b]pyridin-7-yl)-3,8-diazabicyclo[3.2.1]octane-8-carboxylate

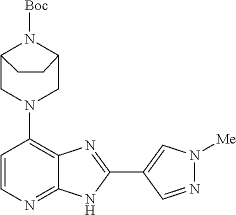 [SMILES: Cn1cc(-c2nc3c(N4CC5CCC(C4)N5C(=O)OC(C)(C)C)ccnc3[nH]2)cn1]

To a solution of tert-butyl 3-(2,3-diaminopyridin-4-yl)-3,8-diazabicyclo[3.2.1]octane-8-carboxylate (Preparation 10, 150.0 mg, 0.470 mmol) in DMF (3.0 mL) was added TsOH (24.3 mg, 0.141 mmol) and 1-methyl-1H-pyrazole-4-carbaldehyde (56.9 mg, 0.517 mmol) and the reaction stirred at 80° C. for 2 h. The cooled reaction was concentrated in vacuo and the residue purified by column chromatography on silica gel (DCM/MeOH=10/1) to give tert-butyl 3-(2-(1-methyl-1H-pyrazol-4-yl)-3H-imidazo[4,5-b]pyridin-7-yl)-3,8-diazabicyclo[3.2.1]octane-8-carboxylate (95.0 mg, 49.4% yield) as a yellow solid.

¹H NMR (400 MHz, MeOH-d₄) δ: 8.22 (s, 1H), 8.06 (s, 1H), 7.89-7.87 (m, 1H), 6.50 (d, 1H), 4.64-4.38 (m, 4H), 3.98 (s, 3H), 3.21-3.18 (m, 2H), 2.00-1.98 (m, 4H), 1.51 (s, 9H).

### Preparation 12

### 7-(3,8-diazabicyclo[3.2.1]octan-3-yl)-2-(1-methyl-1H-pyrazol-4-yl)-3H-imidazo[4,5-b]pyridine hydrochloride

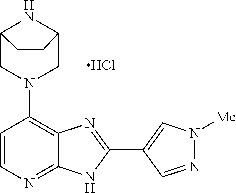 [SMILES: Cn1cc(-c2nc3c(N4CC5CCC(C4)N5)ccnc3[nH]2)cn1 ; Cl]

To a solution of tert-butyl 3-(2-(1-methyl-1H-pyrazol-4-yl)-3H-imidazo[4,5-b]pyridin-7-yl)-3,8-diazabicyclo[3.2.1]octane-8-carboxylate (Preparation 11, 95.0 mg, 0.232 mmol) in DCM (2.0 mL) was added HCl/dioxane (4 M, 5.0 mL) and the reaction stirred at 25° C. for 30 mins. The mixture was evaporated under reduced pressure to give 7-(3,8-diazabicyclo[3.2.1]octan-3-yl)-2-(1-methyl-1H-pyrazol-4-yl)-3H-imidazo[4,5-b]pyridine hydrochloride (70.0 mg, 87.3% yield) as a yellow solid. LCMS m/z=310.2 [M+H]⁺.

### Preparation 13

### 4-chloro-1-tosyl-1H-pyrrolo[2,3-b]pyridine

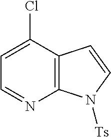 [SMILES: Cc1ccc(S(=O)(=O)n2ccc3c(Cl)ccnc32)cc1]

To a solution of 4-chloro-1H-pyrrolo[2,3-b]pyridine (5.0 g, 32.77 mmol) in DMF (80.0 mL) was added NaH (1.75 g, 60% purity, 39.32 mmol) at 0° C., the mixture stirred at 20° C. for 30 mins, then TsCl (9.4 g, 49.16 mmol) added. The reaction was stirred at 20° C. for 1 h then quenched with water (30 mL) and extracted with EtOAc (20 mL×3). The combined organic layers were washed with brine (20 mL), dried over Na₂SO₄, filtered and concentrated in vacuo. The crude product was purified by column chromatography on silica gel (PE/EtOAc=10/1 to 5/1) to afford 4-chloro-1-tosyl-1H-pyrrolo[2,3-b]pyridine (6.1 g, 60.7% yield) as a white solid. ¹H NMR (400 MHz, CDCl₃) δ: 8.29 (d, 1H), 8.04 (d, 2H), 7.75 (d, 1H), 7.26 (d, 2H), 7.17 (d, 1H), 6.68 (d, 1H), 2.36 (s, 3H).

### Preparation 14

### 4-chloro-2-iodo-1-tosyl-1H-pyrrolo[2,3-b]pyridine

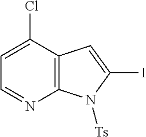 [SMILES: Cc1ccc(S(=O)(=O)n2c(I)cc3c(Cl)ccnc32)cc1]

To a solution of 4-chloro-1-tosyl-1H-pyrrolo[2,3-b]pyridine (Preparation 13, 2.0 g, 6.52 mmol) in THE (50.0 mL) was added LDA (2 M, 3.91 mL) at −70° C. and the solution stirred for 30 mins. I₂ (2.0 g, 7.82 mmol) was added and the reaction stirred at 20° C. for 1 h. The mixture was quenched with water (30 mL) and extracted with EtOAc (20 mL×3). The combined organic layers were washed with brine (20 mL), dried over Na₂SO₄, filtered and concentrated in vacuo. The crude was purified by column chromatography on silica gel (PE/EtOAc=10/1 to 5/1) to give 4-chloro-2-iodo-1-tosyl-1H-pyrrolo[2,3-b]pyridine (800.0 mg, 28.4% yield) as a white solid. ¹H NMR (400 MHz, CDCl₃) δ: 8.27 (d, 1H), 8.09 (d, 2H), 7.28 (d, 2H), 7.15 (d, 1H), 7.10 (s, 1H), 2.38 (s, 3H).

### Preparation 15

### 4-chloro-2-(2-methoxypyridin-4-yl)-1-tosyl-1H-pyrrolo[2,3-b]pyridine

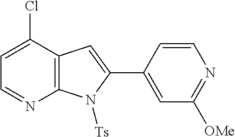 [SMILES: COc1cc(-c2cc3c(Cl)ccnc3n2S(=O)(=O)c2ccc(C)cc2)ccn1]

To a solution of 4-chloro-2-iodo-1-tosyl-1H-pyrrolo[2,3-b]pyridine (Preparation 14, 2.0 g, 4.62 mmol) in dioxane (20 mL) and H₂O (2 mL) was added (2-methoxypyridin-4-yl)boronic acid (848.4 mg, 5.54 mmol), Pd(dppf)Cl₂ (338.2 mg, 0.462 mmol) and K₂CO₃ (1.3 g, 9.24 mmol) at 25° C. and the mixture stirred at 100° C. for 1.5 hours. The reaction mixture was diluted with water (30 mL) and extracted with EtOAc (3×20 mL). The combined organics were washed with brine (30 mL), dried (Na₂SO₄) and evaporated to dryness in vacuo. The residue was purified by SiO₂ chromatography (15/1 to 2/1 PE/EtOAc) give 4-chloro-2-(2-methoxypyridin-4-yl)-1-tosyl-1H-pyrrolo[2,3-b]pyridine as a yellow solid (1.6 g, 83.2%). LCMS m/z=414.2 [M+H]⁺

### Preparation 16

### 4-chloro-2-(1-methyl-1H-pyrazol-4-yl)-1-tosyl-1H-pyrrolo[2,3-b]pyridine

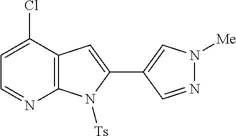 [SMILES: Cc1ccc(S(=O)(=O)n2c(-c3cnn(C)c3)cc3c(Cl)ccnc32)cc1]

To a solution of 4-chloro-2-iodo-1-tosyl-1H-pyrrolo[2,3-b]pyridine (Preparation 14, 500.0 mg, 1.16 mmol) in dioxane (3.0 mL) and H₂O (0.3 mL) were added 1-methylpyrazole-4-boronic acid pinacol ester (289.6 mg, 1.39 mmol), K₂CO₃ (320.7 mg, 2.32 mmol), Pd(dppf)Cl₂ (84.9 mg, 0.116 mmol) and the reaction stirred at 100° C. for 3 h. The cooled mixture was concentrated in vacuo and the residue purified by column chromatography on silica gel (PE/EtOAc=15/1 to 1/1) to afford 4-chloro-2-(1-methyl-1H-pyrazol-4-yl)-1-tosyl-1H-pyrrolo[2,3-b]pyridine (287.0 mg, 64.0% yield) as yellow solid.

¹H NMR (400 MHz, CDCl₃) δ: 8.35-8.30 (m, 1H), 7.75-7.64 (m, 4H), 7.18-7.14 (m, 3H), 6.55 (s, 1H), 4.00 (s, 3H), 2.32 (s, 3H).

### Preparation 17

### tert-butyl 3-(2-(2-methoxypyridin-4-yl)-1-tosyl-1H-pyrrolo[2,3-b]pyridin-4-yl)-3,8-diazabicyclo[3.2.1]octane-8-carboxylate

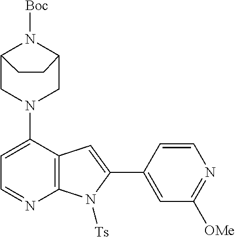 [SMILES: COc1cc(-c2cc3c(N4CC5CCC(C4)N5C(=O)OC(C)(C)C)ccnc3n2S(=O)(=O)c2ccc(C)cc2)ccn1]

To a solution of 4-chloro-2-(2-methoxypyridin-4-yl)-1-tosyl-1H-pyrrolo[2,3-b]pyridine (Preparation 15, 800 mg, 1.93 mmol) and tert-butyl 3,8-diazabicyclo[3.2.1]octane-8-carboxylate (492.4 mg, 2.32 mmol) in t-BuOH (10 mL) was added Cs₂CO₃ (1.3 g, 3.87 mmol) and RuPhos Pd G3 (161.7 mg, 0.193 mmol) at 25° C. and the mixture stirred at 110° C. for 2 h under microwave and N₂. The mixture was diluted with H₂O (10 mL) and extracted with EtOAc (3×15 mL). The combined organics were washed with brine (15 mL), dried (Na₂SO₄) and evaporated to dryness in vacuo. The residue was purified by SiO₂ chromatography (15/1 to 1/1 PE/EtOAc) to give tert-butyl 3-(2-(2-methoxypyridin-4-yl)-1-tosyl-1H-pyrrolo[2,3-b]pyridin-4-yl)-3,8-diazabicyclo[3.2.1]octane-8-carboxylate as a yellow solid (800 mg, 70.5% yield). LCMS m/z=590.3 [M+H]⁺

### Preparation 18

### tert-butyl 3-(2-(1-methyl-1H-pyrazol-4-yl)-1-tosyl-1H-pyrrolo[2,3-b]pyridin-4-yl)-3,8-diazabicyclo[3.2.1]octane-8-carboxylate

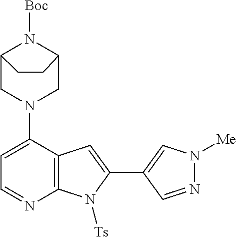 [SMILES: Cc1ccc(S(=O)(=O)n2c(-c3cnn(C)c3)cc3c(N4CC5CCC(C4)N5C(=O)OC(C)(C)C)ccnc32)cc1]

To a solution of 4-chloro-2-(1-methyl-1H-pyrazol-4-yl)-1-tosyl-1H-pyrrolo[2,3-b]pyridine (Preparation 16, 250.0 mg, 0.646 mmol) in t-BuOH (5.0 mL) were added tert-butyl 3,8-diazabicyclo[3.2.1]octane-8-carboxylate (137.2 mg, 0.646 mmol), Ruphos Pd G3 (54.1 mg, 0.065 mmol) and Cs₂CO₃ (421.1 mg, 1.29 mmol) and the reaction stirred at 110° C. for 2 h under microwave irradiation. The mixture was poured into water (20.0 mL) and extracted with EtOAc (20.0 mL×3). The combined organic layers were washed with brine (20.0 mL), dried over Na₂SO₄, filtered and concentrated in vacuo. The residue was purified by column chromatography on silica gel (PE/EtOAc=15/1 to 1/2) to afford tert-butyl 3-(2-(1-methyl-1H-pyrazol-4-yl)-1-tosyl-1H-pyrrolo[2,3-b]pyridin-4-yl)-3,8-diazabicyclo[3.2.1]octane-8-carboxylate (302.0 mg, 83.1% yield) as yellow gum. LCMS m/z=563.7 [M+H]⁺.

### Preparation 19

### tert-butyl 3-(2-(1-methyl-1H-pyrazol-4-yl)-1H-pyrrolo[2,3-b]pyridin-4-yl)-3,8-diazabicyclo[3.2.1]octane-8-carboxylate

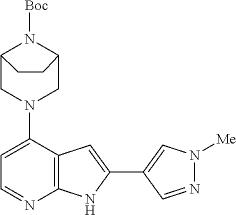 [SMILES: Cn1cc(-c2cc3c(N4CC5CCC(C4)N5C(=O)OC(C)(C)C)ccnc3[nH]2)cn1]

To a solution of tert-butyl 3-(2-(1-methyl-1H-pyrazol-4-yl)-1-tosyl-1H-pyrrolo[2,3-b]pyridin-4-yl)-3,8-diazabicyclo[3.2.1]octane-8-carboxylate (Preparation 18, 280.0 mg, 0.498 mmol) in MeOH (4.0 mL) was added NaOH (5 N, 0.1 mL) and the reaction stirred at 50° C. for 15 h. The mixture was concentrated in vacuo, EtOAc added and the mixture filtered. The filtrate was evaporated under reduced pressure to afford tert-butyl 3-(2-(1-methyl-1H-pyrazol-4-yl)-1H-pyrrolo[2,3-b]pyridin-4-yl)-3,8-diazabicyclo[3.2.1]octane-8-carboxylate (215.0 mg, crude) as yellow gum. LCMS m/z=409.3 [M+H]⁺.

### Preparation 20

### 4-(3,8-diazabicyclo[3.2.1]octan-3-yl)-2-(1-methyl-1H-pyrazol-4-yl)-1H-pyrrolo[2,3-b]pyridine hydrochloride

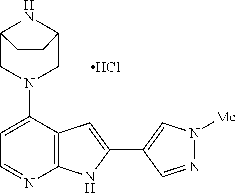 [SMILES: Cn1cc(-c2cc3c(N4CC5CCC(C4)N5)ccnc3[nH]2)cn1 ; Cl]

To a solution of tert-butyl 3-(2-(1-methyl-1H-pyrazol-4-yl)-1H-pyrrolo[2,3-b]pyridin-4-yl)-3,8-diazabicyclo[3.2.1]octane-8-carboxylate (Preparation 19, 215.0 mg, 0.526 mmol) in EtOAc (2.0 mL) was added HCl/EtOAc (4 M, 2.0 mL) and the reaction stirred at 20° C. for 1 h. The mixture was evaporated under reduced pressure to afford 4-(3,8-diazabicyclo[3.2.1]octan-3-yl)-2-(1-methyl-1H-pyrazol-4-yl)-1H-pyrrolo[2,3-b]pyridine hydrochloride (173 mg, 95.3%) as yellow solid. LCMS m/z=309.1 [M+H]⁺.

### Preparation 21

### 4-(3,8-diazabicyclo[3.2.1]octan-3-yl)-2-(2-methoxypyridin-4-yl)-1-tosyl-1H-pyrrolo[2,3-b]pyridine hydrochloride

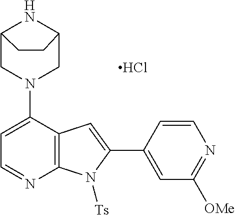 [SMILES: COc1cc(-c2cc3c(N4CC5CCC(C4)N5)ccnc3n2S(=O)(=O)c2ccc(C)cc2)ccn1 ; Cl]

The title compound was prepared as a yellow solid (80 mg) from tert-butyl 3-(2-(2-methoxypyridin-4-yl)-1-tosyl-1H-pyrrolo[2,3-b]pyridin-4-yl)-3,8-diazabicyclo[3.2.1]octane-8-carboxylate (Preparation 17, 100 mg, 0.170 mmol) using an analogous method to that described for Preparation 12. LCMS m/z=490.2 [M+H]⁺

### Preparation 22

### 4-(3,8-diazabicyclo[3.2.1]octan-3-yl)-2-(2-methoxypyridin-4-yl)-1H-pyrrolo[2,3-b]pyridine hydrochloride

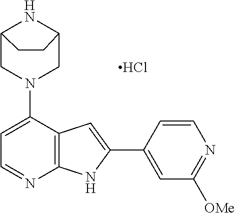 [SMILES: COc1cc(-c2cc3c(N4CC5CCC(C4)N5)ccnc3[nH]2)ccn1 ; Cl]

HCl/EtOAc (4 M, 0.3 mL) was added to a solution of tert-butyl 3-(2-(2-methoxypyridin-4-yl)-1-tosyl-1H-pyrrolo[2,3-b]pyridin-4-yl)-3,8-diazabicyclo[3.2.1]octane-8-carboxylate (Preparation 17, 700 mg, 1.19 mmol) in EtOAc (5 mL) and the mixture was stirred at 20° C. for 1 h. The reaction mixture was evaporated to dryness and the residue was purified by Prep-HPLC-1 (gradient 20-50%) to give 4-(3,8-diazabicyclo[3.2.1]octan-3-yl)-2-(2-methoxypyridin-4-yl)-1H-pyrrolo[2,3-b]pyridine hydrochloride as a yellow solid (280 mg, 63.4%). LCMS m/z=336.4 [M+H]⁺

### Preparation 23

### 1-(4-bromo-1H-pyrrol-2-yl)ethan-1-one

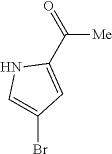

Amberlyst® 15 (3.97 g) was added to a solution of 1-(1H-pyrrol-2-yl)ethan-1-one 1 (44 g, 403 mmol) in THE (442 mL) at rt, the mixture cooled to −30° C. and NBS (71.8 g, 403 mmol) added portion wise. The resulting mixture was stirred for 2 h before warming to rt and filtered. The filtrate was diluted with saturated aq. sodium sulfite and the resulting mixture extracted with DCM (2×). The combined organics were evaporated to dryness in vacuo and the residue dissolved in TBME. The solution was washed with NaHCO₃ (2×), brine, dried (Na₂SO₄) and concentrated under reduced pressure to give 1-(4-bromo-1H-pyrrol-2-yl)ethan-1-one as a white solid which was used without any further purification (76.2 g). LCMS m/z=188.0 [M+H]⁺.

### Preparation 24

### (E)-1-(4-bromo-1H-pyrrol-2-yl)-3-(dimethylamino)prop-2-en-1-one

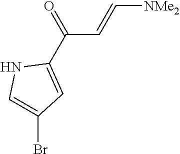 [SMILES: CN(C)/C=C/C(=O)c1cc(Br)c[nH]1]

A solution of 1-(4-bromo-1H-pyrrol-2-yl)ethan-1-one (Preparation 23, 76.2 g, 0.36 mol) in DMF-DMA (390 mL) was heated to 85° C. overnight. The yellow suspension was diluted with heptane and the resulting solid was collected by filtration. The filter cake was washed with heptane and dried to give (E)-1-(4-bromo-1H-pyrrol-2-yl)-3-(dimethylamino)prop-2-en-1-one as a light brown solid (61.5 g, 69%). LCMS m/z=243.1 [M+H]⁺.

### Preparation 25

### 6-bromopyrrolo[1,2-b]pyridazin-4-ol

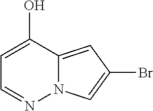 [SMILES: Oc1ccnn2cc(Br)cc12]

KO^{t}Bu (42.6 g, 379 mmol) was added portion wise to a solution of (E)-1-(4-bromo-1H-pyrrol-2-yl)-3-(dimethylamino)prop-2-en-1-one (Preparation 24, 61.5 g, 253 mmol) in NMP (1.85 L) and the mixture stirred at rt for 30 mins. The reaction mixture was cooled on an ice/water bath and O-(4-nitrobenzoyl)hydroxylamine (69.1 g, 379 mmol) was added and the resulting mixture was stirred at 0° C. for 1 h and then overnight at rt. The mixture was cooled to 0° C. and saturated aq. NH₄Cl (500 mL) was added dropwise. The mixture was diluted with H₂O (500 mL) and the pH adjusted to 3-4 with aq. 2 N HCl and extracted with TBME (3×). The combined organics were concentrated to half of the volume and washed with H₂O (2×), brine and then evaporated to dryness in vacuo. The residue was filtered through a pad of silica (0-100% EtOAc/heptane) to afford 6-bromopyrrolo[1,2-b]pyridazin-4-ol (58 g, 54%) which was used without any further purification. LCMS m/z=213.0 [M+H]⁺.

### Preparation 26

### 6-bromopyrrolo[1,2-b]pyridazin-4-yl trifluoromethanesulfonate

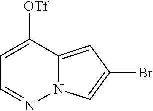 [SMILES: O=S(=O)(Oc1ccnn2cc(Br)cc12)C(F)(F)F]

Trifluoromethanesulfonic anhydride (88.3 g, 313 mmol) was added to a solution of 6-bromopyrrolo[1,2-b]pyridazin-4-ol (Preparation 25, 58 g, −140 mmol, ˜50% pure) and TEA (32.5 g, 321 mmol) in DCM (870 mL) at 0° C. and the mixture stirred for 1 h with cooling and then at rt for 2 h. The mixture was diluted with DCM and washed with Na₂CO₃ (2×), brine, dried (Na₂SO₄) and evaporated to dryness in vacuo. The residue was purified by silica gel chromatography (0-2% TBME/heptane) to give 6-bromopyrrolo[1,2-b]pyridazin-4-yl trifluoromethanesulfonate as a dark oil (21.1 g, 43%). LCMS m/z=344.6 [M+H]⁺.

### Preparation 27

### tert-butyl 3-(6-bromopyrrolo[1,2-b]pyridazin-4-yl)-3,8-diazabicyclo[3.2.1]octane-8-carboxylate

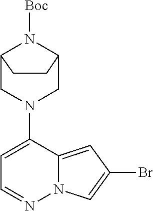 [SMILES: CC(C)(C)OC(=O)N1C2CCC1CN(c1ccnn3cc(Br)cc13)C2]

A solution of 6-bromopyrrolo[1,2-b]pyridazin-4-yl trifluoromethanesulfonate (Preparation 26, 5.0 g, 14.49 mmol), tert-butyl 3,8-diazabicyclo[3.2.1]octane-8-carboxylate (3.4 g, 15.9 mmol) and TEA (2.2 g, 21.7 mmol) in NMP (50 mL) was stirred at 100° C. for 30 mins. The mixture was diluted with water (50 mL) and extracted with EtOAc (3×40 mL). The combined organics were washed with brine (60 mL), dried (Na₂SO₄) and evaporated to dryness in vacuo. The residue was purified by silica gel column chromatography (0-20% EtOAc/PE) to give tert-butyl 3-(6-bromopyrrolo[1,2-b]pyridazin-4-yl)-3,8-diazabicyclo[3.2.1]octane-8-carboxylate as a white solid (4.2 g, 70.3% yield). LCMS m/z=409.2 [M+H]⁺.

### Preparation 28

### tert-butyl 3-(6-(1-methyl-1H-pyrazol-4-yl)pyrrolo[1,2-b]pyridazin-4-yl)-3,8-diazabicyclo[3.2.1]octane-8-carboxylate

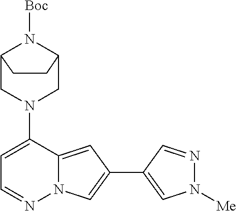 [SMILES: Cn1cc(-c2cc3c(N4CC5CCC(C4)N5C(=O)OC(C)(C)C)ccnn3c2)cn1]

A mixture of tert-butyl 3-(6-bromopyrrolo[1,2-b]pyridazin-4-yl)-3,8-diazabicyclo[3.2.1]octane-8-carboxylate (Preparation 27, 1 eq, 25.42 mmol), 1-methyl-4-(4,4,5,5-tetramethyl-1,3,2-dioxaborolan-2-yl)-1H-pyrazole (1.5 eq, 38.13 mmol), KF (3.0 M, 1 eq, 25.4 mL), and Pd(amphos)Cl₂ (1.80 g, 10 mol %) were suspended in dioxane (127 mL) and the reaction mixture was sparged with N₂ for 5 mins and then heated to 60° C. overnight. The cooled reaction was diluted with EtOAc (500 mL) and washed with saturated NH₄Cl (2×200 mL), water (200 mL) and brine (200 mL). The combined organics were dried (MgSO₄) and evaporated to dryness in vacuo. The residue was purified using silica gel column chromatography (0-70% EtOAc/heptane) to afford tert-butyl 3-(6-(1-methyl-1H-pyrazol-4-yl)pyrrolo[1,2-b]pyridazin-4-yl)-3,8-diazabicyclo[3.2.1]octane-8-carboxylate (10.9 g, 96.3%). LCMS m/z=409.3 [M+H]⁺

### Preparation 29

### 4-(3,8-diazabicyclo[3.2.1]octan-3-yl)-6-(1-methyl-1H-pyrazol-4-yl)pyrrolo[1,2-b]pyridazine hydrochloride

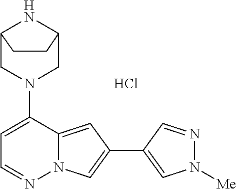 [SMILES: Cn1cc(-c2cc3c(N4CC5CCC(C4)N5)ccnn3c2)cn1 ; Cl]

To tert-butyl 3-(6-(1-methyl-1H-pyrazol-4-yl)pyrrolo[1,2-b]pyridazin-4-yl)-3,8-diazabicyclo[3.2.1]octane-8-carboxylate (Preparation 28, 1.83 g, 4.48 mmol) in DCM (18 mL) was added HCl (4 M, 11.2 mL) and the mixture stirred at rt for 2 h. The reaction mixture was evaporated to dryness in vacuo and dried under high vacuum to give 4-(3,8-diazabicyclo[3.2.1]octan-3-yl)-6-(1-methyl-1H-pyrazol-4-yl)pyrrolo[1,2-b]pyridazine hydrochloride as a light yellow solid (1.685 g). LCMS m/z=309.2 [M+H]⁺.

### Preparation 30

### 4-(8-(2-((tert-butyldimethylsilyl)oxy)cyclobutyl)-3,8-diazabicyclo[3.2.1]octan-3-yl)-6-(1-methyl-1H-pyrazol-4-yl)pyrrolo[1,2-b]pyridazine

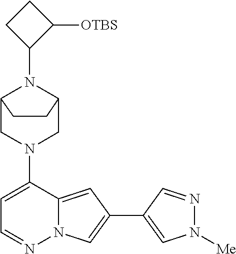 [SMILES: Cn1cc(-c2cc3c(N4CC5CCC(C4)N5C4CCC4O[Si](C)(C)C(C)(C)C)ccnn3c2)cn1]

NaBH₃CN (273 mg, 4.35 mmol) was added to a solution of 4-(3,8-diazabicyclo[3.2.1]octan-3-yl)-6-(1-methyl-1H-pyrazol-4-yl)pyrrolo[1,2-b]pyridazine hydrochloride (Preparation 29, 300 mg, 0.870 mmol) and 2-((tert-butyldimethylsilyl)oxy)cyclobutan-1-one (872 mg, 4.35 mmol) in MeOH (10 mL) and the mixture stirred at 50° C. for 18 h. The mixture was diluted with water (15 mL) and extracted with EtOAc (3×20 mL). The combined organics were washed with brine (3×10 mL), dried (Na₂SO₄) and evaporated to dryness in vacuo give 4-(8-(2-((tert-butyldimethylsilyl)oxy)cyclobutyl)-3,8-diazabicyclo[3.2.1]octan-3-yl)-6-(1-methyl-1H-pyrazol-4-yl)pyrrolo[1,2-b]pyridazine as a yellow oil (428 mg) which was used without further purification. LCMS m/z=379.2 [M+H−TBS]⁺.

### Preparation 31

### 4-(3,8-diazabicyclo[3.2.1]octan-3-yl)-6-bromopyrrolo[1,2-b]pyridazine hydrochloride

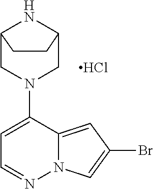 [SMILES: Brc1cc2c(N3CC4CCC(C3)N4)ccnn2c1 ; Cl]

To a solution of tert-butyl 3-(6-bromopyrrolo[1,2-b]pyridazin-4-yl)-3,8-diazabicyclo[3.2.1]octane-8-carboxylate (Preparation 27, 2.3 g, 5.65 mmol) in DCM (20 mL) was added HCl/dioxane (4 M, 15 mL) and the resulting mixture stirred at 25° C. for 3 h. The reaction mixture was evaporated to dryness in vacuo to afford 4-(3,8-diazabicyclo[3.2.1]octan-3-yl)-6-bromopyrrolo[1,2-b]pyridazine hydrochloride as a white solid (3.5 g) which was used without further purification.

### Preparation 32

### 3-(3-(6-bromopyrrolo[1,2-b]pyridazin-4-yl)-3,8-diazabicyclo[3.2.1]octan-8-yl)cyclobutane-1-carbonitrile

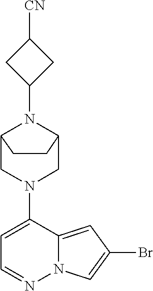 [SMILES: N#CC1CC(N2C3CCC2CN(c2ccnn4cc(Br)cc24)C3)C1]

NaBH₃CN (914.3 mg, 14.55 mmol) was added to a solution of 4-(3,8-diazabicyclo[3.2.1]octan-3-yl)-6-bromopyrrolo[1,2-b]pyridazine hydrochloride (Preparation 31, 1.0 g, 2.91 mmol) and 3-oxocyclobutane-1-carbonitrile (830.2 mg, 8.73 mmol). in MeOH (10 mL) and the mixture stirred at 50° C. for 4 h. The mixture was evaporated to dryness in vacuo and the residue purified by prep-HPLC-3 (gradient 43-73%) to give 3-(3-(6-bromopyrrolo[1,2-b]pyridazin-4-yl)-3,8-diazabicyclo[3.2.1]octan-8-yl)cyclobutane-1-carbonitrile as a white solid (210 mg, 18.7%) as a white solid. LCMS m/z=386.1 [M+H]⁺.

### Preparation 33

### (1S,3s)-3-(3-(6-bromopyrrolo[1,2-b]pyridazin-4-yl)-3,8-diazabicyclo[3.2.1]octan-8-yl)cyclobutane-1-carbonitrile

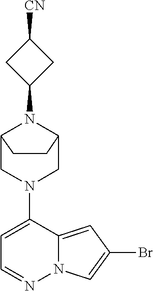 [SMILES: N#C[C@H]1C[C@@H](N2C3CCC2CN(c2ccnn4cc(Br)cc24)C3)C1]

*Stereochemistry assigned by NOE studies

3-(3-(6-bromopyrrolo[1,2-b]pyridazin-4-yl)-3,8-diazabicyclo[3.2.1]octan-8-yl)cyclobutane-1-carbonitrile (Preparation 32, 210 mg, 0.544 mmol) was separated by SFC-1 (45% EtOH) to give (1S,3s)-3-(3-(6-bromopyrrolo[1,2-b]pyridazin-4-yl)-3,8-diazabicyclo[3.2.1]octan-8-yl)cyclobutane-1-carbonitrile as a white solid (130 mg, 61.9%). ¹H NMR (400 MHz, CDCl₃) δ: 7.77 (d, 1H), 7.62 (s, 1H), 6.50 (s, 1H), 5.74 (d, 1H), 3.64-3.60 (m, 2H), 3.28-3.02 (m, 5H), 2.85-2.78 (m, 1H), 2.76-2.53 (m, 2H), 2.48-2.31 (m, 2H), 2.00-1.89-2 (m, 4H).

### Preparation 34

### 6-bromo-4-(8-(3,3-difluorocyclobutyl)-3,8-diazabicyclo[3.2.1]octan-3-yl)pyrrolo[1,2-b]pyridazine

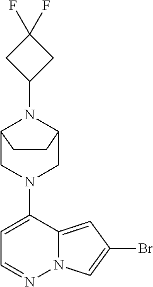 [SMILES: FC1(F)CC(N2C3CCC2CN(c2ccnn4cc(Br)cc24)C3)C1]

TEA (132 mg, 1.30 mmol) was added to a solution of 6-bromopyrrolo[1,2-b]pyridazin-4-yl trifluoromethanesulfonate (Preparation 26, 150 mg, 0.435 mmol) and 8-(3,3-difluorocyclobutyl)-3,8-diazabicyclo[3.2.1]octane dihydrochloride (Preparation 8, 119.6 mg, 0.435 mmol) in DMF (5 mL) and the mixture stirred at 90° C. for 1 h. The mixture was poured into H₂O (30 mL) and extracted with EtOAc (3×20 mL). The combined organics were washed with brine (30 mL), dried (Na₂SO₄) and evaporated to dryness in vacuo. The residue was purified by column chromatography (0-16% EtOAc/PE) to give 6-bromo-4-(8-(3,3-difluorocyclobutyl)-3,8-diazabicyclo[3.2.1]octan-3-yl)pyrrolo[1,2-b]pyridazine as a brown oil (165 mg, 95.6%). ¹H NMR (500 MHz, CDCl₃) δ: 7.77 (d, 1H), 7.61 (d, 1H), 6.51 (d, 1H), 5.74 (d, 1H), 3.64-3.62 (m, 2H), 3.30-3.28 (m, 2H), 3.19-3.16 (m, 2H), 2.92-2.90 (m, 1H), 2.73-2.71 (m, 2H), 2.46-2.43 (m, 2H), 1.99-1.86 (m, 4H).

### Preparation 35

### tert-butyl 3-(6-(4,4,5,5-tetramethyl-1,3,2-dioxaborolan-2-yl)pyrrolo[1,2-b]pyridazin-4-yl)-3,8-diazabicyclo[3.2.1]octane-8-carboxylate

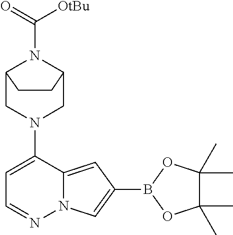 [SMILES: CC(C)(C)OC(=O)N1C2CCC1CN(c1ccnn3cc(B4OC(C)(C)C(C)(C)O4)cc13)C2]

A mixture of tert-butyl 3-(6-bromopyrrolo[1,2-b]pyridazin-4-yl)-3,8-diazabicyclo[3.2.1]octane-8-carboxylate (Preparation 27, 598.73 mg, 1.47 mmol), bis(pinacolato)diboron (746.58 mg, 2.94 mmol), KOAc (432.80 mg, 4.41 mmol) and Pd(dppf)Cl₂ (120.05 mg, 0.147 mmol) in MeCN (2.94 mL) was purged with N₂ and stirred at 65° C. overnight under N₂. The mixture was cooled to rt, concentrated in vacuo and the crude purified by column chromatography (heptane/EtOAc=0-50%) to afford tert-butyl 3-(6-(4,4,5,5-tetramethyl-1,3,2-dioxaborolan-2-yl)pyrrolo[1,2-b]pyridazin-4-yl)-3,8-diazabicyclo[3.2.1]octane-8-carboxylate (560.0 mg, 83.8% yield) as a white solid. LCMS m/z=455.3 [M+H]⁺.

### Preparation 36

### tert-butyl 3-(6-(6-chloropyridazin-4-yl)pyrrolo[1,2-b]pyridazin-4-yl)-3,8-diazabicyclo[3.2.1]octane-8-carboxylate

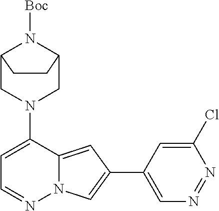 [SMILES: CC(C)(C)OC(=O)N1C2CCC1CN(c1ccnn3cc(-c4cnnc(Cl)c4)cc13)C2]

A mixture of tert-butyl 3-(6-(4,4,5,5-tetramethyl-1,3,2-dioxaborolan-2-yl)pyrrolo[1,2-b]pyridazin-4-yl)-3,8-diazabicyclo[3.2.1]octane-8-carboxylate (Preparation 35, 563.4 mg, 1.24 mmol), 3,5-dichloropyridazine (277.1 mg, 1.86 mmol), Pd(amphos)Cl₂ (61.5 mg, 0.087 mmol) and KF (3.0 M, 1.24 mL) were dissolved in dioxane (6.20 mL) and the reaction mixture was purged with N₂ for 5 mins, then heated to 80° C. for 4 h. The cooled mixture was diluted with EtOAc, washed with NH₄Cl (2×) and brine, dried over MgSO₄, filtered and concentrated in vacuo. The crude residue was purified by automated silica gel chromatography (10 to 55% EtOAc/heptane) to provide tert-butyl 3-(6-(6-chloropyridazin-4-yl)pyrrolo[1,2-b]pyridazin-4-yl)-3,8-diazabicyclo[3.2.1]octane-8-carboxylate (287 mg, 52.5%) as a yellow solid. LCMS m/z=441.2 [M+H]⁺.

### Preparation 37

### tert-butyl 3-(6-(pyridazin-4-yl)pyrrolo[1,2-b]pyridazin-4-yl)-3,8-diazabicyclo[3.2.1]octane-8-carboxylate

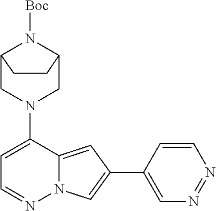 [SMILES: CC(C)(C)OC(=O)N1C2CCC1CN(c1ccnn3cc(-c4ccnnc4)cc13)C2]

A solution of 6-bromopyrrolo[1,2-b]pyridazin-4-yl trifluoromethanesulfonate (Preparation 26, 150 mg, 0.368 mmol), 4-(tributylstannyl)pyridazine (204 mg, 0.552 mmol), CuCl (4.4 mg, 0.0442 mmol), CsF (167.8 mg, 1.10 mmol) and Pd(PPh₃)₄ (42.6 mg, 0.0368 mmol) in DMF (5 mL) under N₂ was heated at 150° C. for 1 h under microwave. The mixture was poured into water (10 mL) and extracted with EtOAc (2×10 mL). The combined extracts were washed with brine (2 10 mL), dried (Na₂SO₄) and evaporated to dryness in vacuo. The residue was purified by column chromatography (PE/EtOAc=20/1 to 1/1) to give tert-butyl 3-(6-(pyridazin-4-yl)pyrrolo[1,2-b]pyridazin-4-yl)-3,8-diazabicyclo[3.2.1]octane-8-carboxylate as a yellow oil (80 mg, 53.4%). ¹H NMR (400 MHz, CDCl₃) δ: 9.47 (s, 1H), 9.13 (d, 1H), 8.08 (d, 1H), 7.87 (d, 1H), 7.62-7.64 (m, 1H), 6.82 (d, 1H), 5.84 (d, 1H), 4.44-4.53 (m, 2H), 3.60-3.74 (m, 2H), 3.27-3.36 (m, 2H), 1.98-2.09 (m, 4H), 1.50 (s, 9H).

### Preparation 38

### tert-butyl 3-(6-(6-methoxypyridazin-4-yl)pyrrolo[1,2-b]pyridazin-4-yl)-3,8-diazabicyclo[3.2.1]octane-8-carboxylate

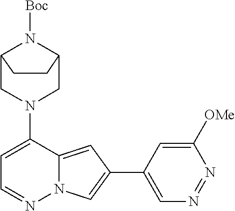 [SMILES: COc1cc(-c2cc3c(N4CC5CCC(C4)N5C(=O)OC(C)(C)C)ccnn3c2)cnn1]

To tert-butyl 3-(6-(6-chloropyridazin-4-yl)pyrrolo[1,2-b]pyridazin-4-yl)-3,8-diazabicyclo[3.2.1]octane-8-carboxylate (Preparation 36, 46 mg, 0.104 mmol) in MeOH/DMSO (504 μL/500 μL) was added NaOMe (25% wt in MeOH, 239 μL 1.04 mmol) and the reaction heated to 50° C. overnight. The cooled reaction was diluted with sat. aq NH₄Cl and EtOAc, the layers separated and the aqueous phase extracted with EtOAc (2×). The combined organic layers were washed with brine, dried (MgSO₄), filtered, and evaporated under reduced pressure to afford tert-butyl 3-(6-(6-methoxypyridazin-4-yl)pyrrolo[1,2-b]pyridazin-4-yl)-3,8-diazabicyclo[3.2.1]octane-8-carboxylate. LCMS m/z=437.3 [M+H]⁺.

### Preparation 39

### tert-butyl 3-(6-(6-methylpyridazin-4-yl)pyrrolo[1,2-b]pyridazin-4-yl)-3,8-diazabicyclo[3.2.1]octane-8-carboxylate

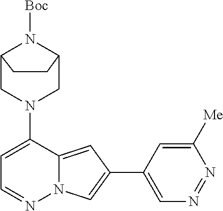 [SMILES: Cc1cc(-c2cc3c(N4CC5CCC(C4)N5C(=O)OC(C)(C)C)ccnn3c2)cnn1]

To a solution of tert-butyl 3-(6-(6-chloropyridazin-4-yl)pyrrolo[1,2-b]pyridazin-4-yl)-3,8-diazabicyclo[3.2.1]octane-8-carboxylate (Preparation 36, 100 mg, 0.227 mmol) and methylboronic acid (20.4 mg, 0.340 mmol) in dioxane (10 mL) and H₂O (1 mL) was added K₂CO₃ (94.0 mg, 0.680 mmol) and Pd(dppf)Cl₂ (16.6 mg, 0.0227 mmol) at 25° C. and the resulting mixture stirred at 100° C. for 16 hours. The mixture was diluted with H₂O (20 mL) and extracted with EtOAc (3×10 mL). The combined organics were washed by brine (20 mL), dried (Na₂SO₄) and evaporated to dryness in vacuo. The residue was purified by column chromatography (PE/EtOAc=15/1 to 1/1) to give tert-butyl 3-(6-(6-methylpyridazin-4-yl)pyrrolo[1,2-b]pyridazin-4-yl)-3,8-diazabicyclo[3.2.1]octane-8-carboxylate as a yellow solid (70 mg, 73.4%). LCMS m/z=421.4. [M+H]⁺; ¹H NMR (500 MHz, CDCl₃) δ: 9.31 (s, 1H), 8.06 (d, 1H), 7.86 (d, 1H), 7.48 (s, 1H), 6.81 (s, 1H), 5.83 (d, 1H), 4.44-4.49 (m, 2H), 3.65-3.80 (m, 2H), 3.25-3.30 (m, 2H), 2.76 (s, 3H), 2.06-2.10 (m, 4H), 1.50 (s, 9H).

### Preparation 40

### 4-(3,8-diazabicyclo[3.2.1]octan-3-yl)-6-(6-methoxypyridazin-4-yl)pyrrolo[1,2-b]pyridazine hydrochloride

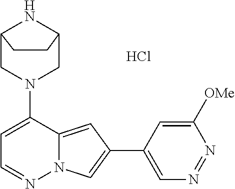 [SMILES: COc1cc(-c2cc3c(N4CC5CCC(C4)N5)ccnn3c2)cnn1 ; Cl]

To a solution of tert-butyl 3-(6-(6-methoxypyridazin-4-yl)pyrrolo[1,2-b]pyridazin-4-yl)-3,8-diazabicyclo[3.2.1]octane-8-carboxylate (Preparation 38, 73.0 mg, 0.167 mmol) in DCM (1.50 mL) was added 4M HCl (334.5 μL) and the reaction stirred at rt for 30 mins. The mixture was evaporated under reduced pressure to afford 4-(3,8-diazabicyclo[3.2.1]octan-3-yl)-6-(6-methoxypyridazin-4-yl)pyrrolo[1,2-b]pyridazine hydrochloride. LCMS m/z=337.2 [M+H]⁺.

### Preparation 41

### 4-(3,8-diazabicyclo[3.2.1]octan-3-yl)-6-(pyridazin-4-yl)pyrrolo[1,2-b]pyridazine hydrochloride

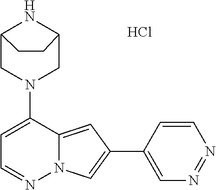 [SMILES: c1cc(-c2cc3c(N4CC5CCC(C4)N5)ccnn3c2)cnn1 ; Cl]

The title compound was prepared as a yellow solid (70 mg) from tert-butyl 3-(6-(pyridazin-4-yl)pyrrolo[1,2-b]pyridazin-4-yl)-3,8-diazabicyclo[3.2.1]octane-8-carboxylate (Preparation 37, 80 mg, 0.197 mmol) using an analogous method to that described for Preparation 48. LCMS m/z=307.2 [M+H]⁺

### Preparation 42

### 4-(3,8-diazabicyclo[3.2.1]octan-3-yl)-6-(6-methylpyridazin-4-yl)pyrrolo[1,2-b]pyridazine hydrochloride

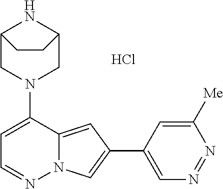 [SMILES: Cc1cc(-c2cc3c(N4CC5CCC(C4)N5)ccnn3c2)cnn1 ; Cl]

The title compound was prepared as a brown solid (70 mg) from tert-butyl 3-(6-(6-methylpyridazin-4-yl)pyrrolo[1,2-b]pyridazin-4-yl)-3,8-diazabicyclo[3.2.1]octane-8-carboxylate (Preparation 39, 70 mg, 0.166 mmol) using an analogous method to that described for Preparation 48. LCMS m/z=321.1 [M+H]⁺

### Preparation 43

### 6-(1-methyl-1H-pyrazol-4-yl)pyrrolo[2,1-f][1,2,4]triazin-4-ol

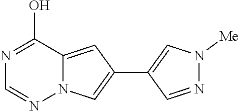 [SMILES: Cn1cc(-c2cc3c(O)ncnn3c2)cn1]

To a solution of 6-bromopyrrolo[2,1-f][1,2,4]triazin-4-ol in dioxane (70 mL), EtOH (15 mL) and H₂O (10 mL) was added 1-methyl-4-(4,4,5,5-tetramethyl-1,3,2-dioxaborolan-2-yl)-1H-pyrazole (1.94 g, 9.34 mmol), Cs₂CO₃ (4.6 g, 14.02 mmol), Pd(dppf)Cl₂ (341.9 mg, 0.467 mmol) and the mixture stirred at 110° C. for 15 h under N₂. The reaction was diluted with H₂O (50 mL) and extracted with EtOAc (3×80 mL). The combined organics were washed with brine (50 mL), dried (Na₂SO₄) and evaporated to dryness in vacuo. The residue was purified by column chromatography (15:1 to 1:2 PE/EtOAc) to give 6-(1-methyl-1H-pyrazol-4-yl)pyrrolo[2,1-f][1,2,4]triazin-4-ol as a yellow solid (300 mg, 29.8%). ¹H NMR (500 MHz, DMSO-d₆) δ: 11.65 (brs, 1H), 8.01-7.99 (m, 1H), 7.83-7.80 (m, 2H), 7.76 (s, 1H), 7.06 (d, 1H), 3.83 (s, 3H).

### Preparation 44

### 4-chloro-6-(1-methyl-1H-pyrazol-4-yl)pyrrolo[2,1-f][1,2,4]triazine

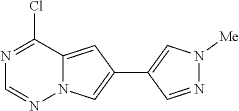 [SMILES: Cn1cc(-c2cc3c(Cl)ncnn3c2)cn1]

A solution of 6-(1-methyl-1H-pyrazol-4-yl)pyrrolo[2,1-f][1,2,4]triazin-4-ol (Preparation 43, 300 mg, 1.39 mmol) in POCl₃ (3 mL) was stirred at 100° C. for 1 h. The reaction mixture was evaporate and the residue dissolved in DCM (20 mL) the pH adjusted to pH ˜7 with aqueous NaHCO₃. The organic layer was dried (Na₂SO₄) and evaporated to dryness in vacuo to give 4-chloro-6-(1-methyl-1H-pyrazol-4-yl)pyrrolo[2,1-f][1,2,4]triazine as a white solid (300 mg, crude) which was used to next step without further purification. LCMS m/z=234.0 [M+H]⁺.

### Preparation 45

### tert-butyl 3-(6-(1-methyl-1H-pyrazol-4-yl)pyrrolo[2,1-f][1,2,4]triazin-4-yl)-3,8-diazabicyclo[3.2.1]octane-8-carboxylate

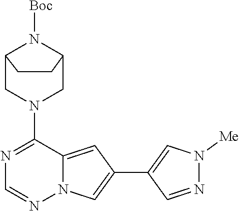 [SMILES: Cn1cc(-c2cc3c(N4CC5CCC(C4)N5C(=O)OC(C)(C)C)ncnn3c2)cn1]

tert-Butyl 3,8-diazabicyclo[3.2.1]octane-8-carboxylate (363.4 mg, 1.71 mmol) was added to a solution of 4-chloro-6-(1-methyl-1H-pyrazol-4-yl)pyrrolo[2,1-f][1,2,4]triazine (Preparation 44, 200 mg, 0.856 mmol) and DIPEA (332 mg, 2.57 mmol) in DMF (5 mL) and the resulting stirred at 25° C. for 1 h. The mixture was diluted with H₂O (20 mL), extracted with EtOAc (2×20 mL). The combined organics were washed with brine (2×20 mL), dried (Na₂SO₄) and evaporated to dryness in vacuo. The residue was purified by chromatography on silica gel (15:1 to 1:1, PE/EtOAc) to give tert-butyl 3-(6-(1-methyl-1H-pyrazol-4-yl)pyrrolo[2,1-f][1,2,4]triazin-4-yl)-3,8-diazabicyclo[3.2.1]octane-8-carboxylate as a white solid (210 mg, 59.9%). LCMS m/z=410.2 [M+H]⁺.

### Preparation 46

### 4-(3,8-diazabicyclo[3.2.1]octan-3-yl)-6-(1-methyl-1H-pyrazol-4-yl)pyrrolo[2,1-f][1,2,4]triazine hydrochloride

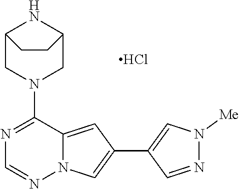 [SMILES: Cn1cc(-c2cc3c(N4CC5CCC(C4)N5)ncnn3c2)cn1 ; Cl]

A solution of tert-butyl 3-(6-(1-methyl-1H-pyrazol-4-yl)pyrrolo[2,1-f][1,2,4]triazin-4-yl)-3,8-diazabicyclo[3.2.1]octane-8-carboxylate (Preparation 45, 200 mg, 0.488 mmol) in HCl/EtOAc (4 M, 5.0 mL) was stirred at 25° C. for 1 h. The mixture was evaporated to dryness to afford 4-(3,8-diazabicyclo[3.2.1]octan-3-yl)-6-(1-methyl-1H-pyrazol-4-yl)pyrrolo[2,1-f][1,2,4]triazine hydrochloride as a white solid (180.0 mg) which was used without further purification. LCMS m/z=310.2 [M+H]⁺.

### Preparation 47

### 4-chloro-7-tosyl-7H-pyrrolo[2,3-d]pyrimidine

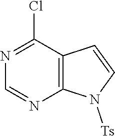 [SMILES: Cc1ccc(S(=O)(=O)n2ccc3c(Cl)ncnc32)cc1]

To a solution of 4-chloro-7H-pyrrolo[2,3-d]pyrimidine (25.0 g, 13.02 mmol) in acetone (250 mL) was added TsCl (37.2 g, 195.4 mmol) and 2M NaOH (97.6 mL) at 0° C. and the reaction stirred at 25° C. for 3 h. The solid was filtered, washed with acetone/H₂O (v/v=1/1), collected and dried under vacuum to afford 4-chloro-7-tosyl-7H-pyrrolo[2,3-d]pyrimidine (45.6 g, 91.0% yield) as a white solid.

¹H NMR (400 MHz, CDCl₃) δ: 8.76 (s, 1H), 8.08 (d, 2H), 7.77 (d, 1H), 4.32 (d, 2H), 6.70 (d, 1H), 2.40 (s, 3H).

### Preparation 48

### 4-chloro-6-iodo-7-tosyl-7H-pyrrolo[2,3-d]pyrimidine

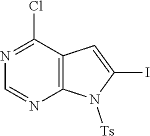 [SMILES: Cc1ccc(S(=O)(=O)n2c(I)cc3c(Cl)ncnc32)cc1]

To a solution of 4-chloro-7-tosyl-7H-pyrrolo[2,3-d]pyrimidine (Preparation 47, 10 g, 32.5 mmol) in THE (200 mL) was added LDA (2 M, 24.37 mL) drop wise at −78° C. under N₂ and the mixture stirred at −78° C. for 1 h. A solution of I₂ (10.7 g, 42.2 mmol) in THE (50 mL) was added drop wise and the mixture stirred at −78° C. for 1 h. The reaction was quenched using sat. aq. NH₄Cl (5.0 mL), diluted with H₂O (10.0 mL) and extracted with EtOAc (20 mL×3). The combined organic extracts were washed with brine (10.0 mL), dried over Na₂SO₄, filtered, and the filtrate concentrated in vacuo. The crude was purified by column chromatography on silica gel (PE/EtOAc=10/1 to 3/1) to afford 4-chloro-6-iodo-7-tosyl-7H-pyrrolo[2,3-d]pyrimidine (6.0 g, 42.6% yield) as a yellow solid. LCMS m/z=433.9 [M+H]⁺.

### Preparation 49

### 4-chloro-6-(1-methyl-1H-pyrazol-4-yl)-7-tosyl-7H-pyrrolo[2,3-d]pyrimidine

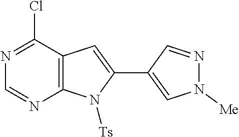 [SMILES: Cc1ccc(S(=O)(=O)n2c(-c3cnn(C)c3)cc3c(Cl)ncnc32)cc1]

Pd(dppf)Cl₂ (143.4 mg, 0.196 mmol) was added to a solution of 4-chloro-6-iodo-7-tosyl-7H-pyrrolo[2,3-d]pyrimidine (Preparation 48, 850 mg, 1.96 mmol), 1-methyl-4-(4,4,5,5-tetramethyl-1,3,2-dioxaborolan-2-yl)-1H-pyrazole (612 mg, 2.94 mmol) and K₃PO₄ (832 mg, 3.92 mmol) in dioxane (3 mL) and H₂O (0.3 mL) at 25° C. under N₂ and the mixture stirred at 30° C. for 12 h. The reaction was diluted with H₂O (10 mL) and extracted with EtOAc (3×10 mL). The combine organics were washed with brine (20 mL), dried (Na₂SO₄) and evaporated to dryness. The residue was purified by column chromatography on SiO₂ (15:1 to 1:1, PE/EtOAc) to give 4-chloro-6-(1-methyl-1H-pyrazol-4-yl)-7-tosyl-7H-pyrrolo[2,3-d]pyrimidine as a pink solid (210 mg, 27.6%). ¹H NMR (500 MHz, CDCl₃) δ: 8.79 (s, 1H), 7.77-7.74 (m, 3H), 7.64 (s, 1H), 7.22 (d, 2H), 6.55 (s, 1H), 4.03 (s, 3H), 2.37 (s, 3H).

### Preparation 50

### tert-butyl 3-(6-(1-methyl-1H-pyrazol-4-yl)-7-tosyl-7H-pyrrolo[2,3-d]pyrimidin-4-yl)-3,8-diazabicyclo[3.2.1]octane-8-carboxylate

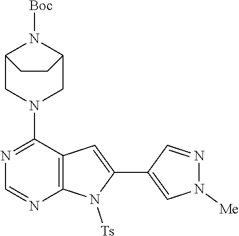 [SMILES: Cc1ccc(S(=O)(=O)n2c(-c3cnn(C)c3)cc3c(N4CC5CCC(C4)N5C(=O)OC(C)(C)C)ncnc32)cc1]

The title compound was prepared as a yellow gum (230 mg, 75.4%) from tert-butyl 3,8-diazabicyclo[3.2.1]octane-8-carboxylate and 4-chloro-6-(1-methyl-1H-pyrazol-4-yl)-7-tosyl-7H-pyrrolo[2,3-d]pyrimidine (Preparation 49) using an analogous method to that described for Preparation 45. LCMS m/z=564.7 [M+H]⁺

### Preparation 51

### tert-butyl 3-(6-(1-methyl-1H-pyrazol-4-yl)-7H-pyrrolo[2,3-d]pyrimidin-4-yl)-3,8-diazabicyclo[3.2.1]octane-8-carboxylate

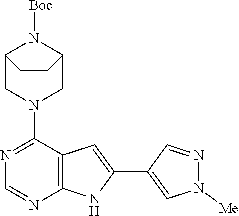 [SMILES: Cn1cc(-c2cc3c(N4CC5CCC(C4)N5C(=O)OC(C)(C)C)ncnc3[nH]2)cn1]

K₂CO₃ (161.8 mg, 1.17 mmol) was added to a solution of tert-butyl (1R,5S)-3-(6-(1-methyl-1H-pyrazol-4-yl)-7-tosyl-7H-pyrrolo[2,3-d]pyrimidin-4-yl)-3,8-diazabicyclo[3.2.1]octane-8-carboxylate (Preparation 50, 220 mg, 0.390 mmol) in MeOH (8 mL) and the mixture stirred at 50° C. for 4 h. The reaction was concentrated under reduced pressure and the residue was purified by column chromatography on SiO₂ (25%-100% EtOAc/PE) to give tert-butyl 3-(6-(1-methyl-1H-pyrazol-4-yl)-7H-pyrrolo[2,3-d]pyrimidin-4-yl)-3,8-diazabicyclo[3.2.1]octane-8-carboxylate as a yellow gum (149 mg, 93.2%). LCMS m/z=410.2 [M+H]⁺

### Preparation 52

### 4-(3,8-diazabicyclo[3.2.1]octan-3-yl)-6-(1-methyl-1H-pyrazol-4-yl)-7H-pyrrolo[2,3-d]pyrimidine hydrochloride

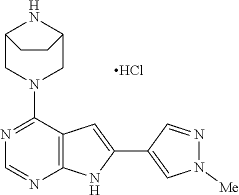 [SMILES: Cn1cc(-c2cc3c(N4CC5CCC(C4)N5)ncnc3[nH]2)cn1 ; Cl]

The title compound was prepared from tert-butyl 3-(6-(1-methyl-1H-pyrazol-4-yl)-7H-pyrrolo[2,3-d]pyrimidin-4-yl)-3,8-diazabicyclo[3.2.1]octane-8-carboxylate (Preparation 51, 140 mg, 0.341 mmol) using an analogous method to that described for Preparation 46. Yield: 160 mg; LCMS m/z=309.7 [M+H]⁺.

### Preparation 53

### 2-bromo-8-hydroxyimidazo[1,2-b]pyridazine

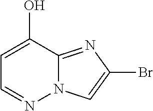 [SMILES: Oc1ccnn2cc(Br)nc12]

A mixture of 2-bromo-8-hydroxyimidazo[1,2-b]pyridazine-7-carboxylic acid (Intermediate 1, step 5, WO2014/039595, 300 mg, 1.16 mmol) in conc. HCl (5 mL, 36% purity) was stirred at 100° C. for 15 h. The cooled mixture was evaporated under reduced pressure and the residue purified by prep-HPLC-2 (gradient 17-47%) to give 2-bromo-8-hydroxyimidazo[1,2-b]pyridazine (60.0 mg, 24.1% yield) as white solid. LCMS m/z=214.2, 216.2 [M+H]⁺.

### Preparation 54

### 2-bromo-8-chloroimidazo[1,2-b]pyridazine

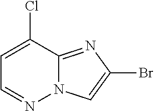 [SMILES: Clc1ccnn2cc(Br)nc12]

2-Bromo-8-hydroxyimidazo[1,2-b]pyridazine (Preparation 53, 20.0 mg, 0.093 mmol) and DIPEA (36.2 mg, 0.28 mmol) were added to POCl₃ (3.29 g, 21.46 mmol) slowly and the reaction stirred at 120° C. for 3 h. Further POCl₃ (10.0 mL) was added and the reaction stirred at 120° C. for an additional 15 h. The cooled reaction was concentrated in vacuo, quenched with H₂O (1.0 mL) and then purified by prep-HPLC-2 (gradient 25-55%) to give 2-bromo-8-chloroimidazo[1,2-b]pyridazine (21.0 mg, 96.7% yield) as yellow solid. LCMS m/z=234.0 [M+H]⁺.

### Preparation 55

### tert-butyl 3-(2-bromoimidazo[1,2-b]pyridazin-8-yl)-3,8-diazabicyclo[3.2.1]octane-8-carboxylate

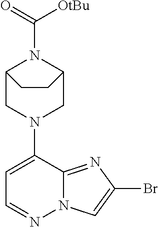 [SMILES: CC(C)(C)OC(=O)N1C2CCC1CN(c1ccnn3cc(Br)nc13)C2]

To a solution of 2-bromo-8-chloroimidazo[1,2-b]pyridazine (Preparation 54, 21.0 mg, 0.090 mmol) in n-BuOH (3.0 mL) was added tert-butyl 3,8-diazabicyclo[3.2.1]octane-8-carboxylate (24.9 mg, 0.117 mmol) and DIPEA (46.7 mg, 0.361 mmol) and the mixture stirred at 130° C. for 1 h under microwave irradiation. The cooled mixture was concentrated in vacuo and the residue purified by column chromatography on silica gel (PE/EtOAc=15/1 to 3/1) to give tert-butyl 3-(2-bromoimidazo[1,2-b]pyridazin-8-yl)-3,8-diazabicyclo[3.2.1]octane-8-carboxylate (25 mg, 67.8% yield) as colorless gum. LCMS m/z=410.1 [M+H]⁺.

### Preparation 56

### tert-butyl 3-(2-(1-methyl-1H-pyrazol-4-yl)imidazo[1,2-b]pyridazin-8-yl)-3,8-diazabicyclo[3.2.1]octane-8-carboxylate

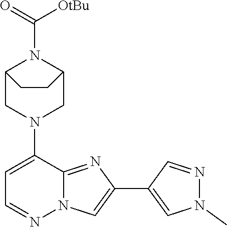 [SMILES: Cn1cc(-c2cn3nccc(N4CC5CCC(C4)N5C(=O)OC(C)(C)C)c3n2)cn1]

tert-Butyl 3-(2-(1-methyl-1H-pyrazol-4-yl)imidazo[1,2-b]pyridazin-8-yl)-3,8-diazabicyclo[3.2.1]octane-8-carboxylate was obtained as a white solid, 105 mg, 69.8% yield, from tert-butyl 3-(2-bromoimidazo[1,2-b]pyridazin-8-yl)-3,8-diazabicyclo[3.2.1]octane-8-carboxylate (Preparation 55) and 1-methyl-4-(4,4,5,5-tetramethyl-1,3,2-dioxaborolan-2-yl)-1H-pyrazole, following the procedure described in Preparation 28. LCMS m/z=410.2 [M+H]⁺.

### Preparation 57

### 8-(3,8-diazabicyclo[3.2.1]octan-3-yl)-2-(1-methyl-1H-pyrazol-4-yl)imidazo[1,2-b]pyridazine hydrochloride

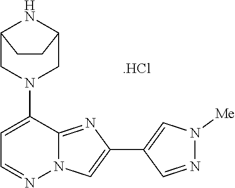 [SMILES: Cn1cc(-c2cn3nccc(N4CC5CCC(C4)N5)c3n2)cn1 ; ".HCl"]

To a solution of tert-butyl 3-(2-(1-methyl-1H-pyrazol-4-yl)imidazo[1,2-b]pyridazin-8-yl)-3,8-diazabicyclo[3.2.1]octane-8-carboxylate (Preparation 56, 105.0 mg, 0.256 mmol) in DCM (2.0 mL) was added HCl/dioxane (4 M, 4 mL) and the mixture stirred at 20° C. for 2 h. The mixture was evaporated under reduced pressure to give 8-(3,8-diazabicyclo[3.2.1]octan-3-yl)-2-(1-methyl-1H-pyrazol-4-yl)imidazo[1,2-b]pyridazine hydrochloride (89.0 mg, crude) as yellow solid. LCMS m/z=310.2 [M+H]⁺.

### EXAMPLES

### Example 1 and 2

### (1R,3r)-1-methyl-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile and (1S,3s)-1-methyl-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile

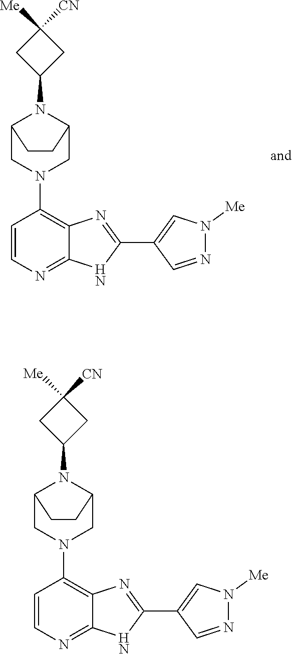 [SMILES: Cn1cc(-c2nc3c(N4CC5CCC(C4)N5[C@H]4C[C@](C)(C#N)C4)ccnc3[nH]2)cn1 ; Cn1cc(-c2nc3c(N4CC5CCC(C4)N5[C@H]4C[C@@](C)(C#N)C4)ccnc3[nH]2)cn1]

* Relative stereochemistry arbitrarily assigned

Part 1. NaBH₃CN (27.3 mg, 0.434 mmol) was added to a solution of 7-(3,8-diazabicyclo[3.2.1]octan-3-yl)-2-(1-methyl-1H-pyrazol-4-yl)-3H-imidazo[4,5-b]pyridine hydrochloride (Preparation 12, 50 mg, 0.145 mmol) and 3,3-dimethylcyclobutan-1-one (78.9 mg, 0.723 mmol) in MeOH (2 mL) and the resulting mixture stirred at 50° C. for 18 h.

The mixture was concentrated and the residue was purified by prep-HPLC-3 (gradient 20-50%) to give 1-methyl-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile as a white solid (21 mg, 36.1%). LCMS m/z=403.2 [M+H]⁺.

Part 2. 1-methyl-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile (Part 1, 21 mg, 0.0522 mmol) was separated by SFC chromatography (SFC-1, 45% EtOH) to give the title compounds.

*Peak 1, Example 1; (1R,3r)-1-methyl-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile (6.1 mg, 29%). LCMS m/z=403.3 [M+H]⁺; ¹H NMR (500 MHz, MeOH-d₄) δ: 8.20 (s, 1H), 8.05 (s, 1H), 7.86 (d, 1H), 6.45 (d, 1H), 4.56-4.51 (m, 2H), 3.98 (s, 3H), 3.37-3.31 (m, 3H), 3.23-3.20 (m, 2H), 2.71-2.69 (m, 2H), 2.09-1.99 (m, 4H), 1.89-1.87 (m, 2H), 1.54 (s, 3H).

*Peak 2, Example 2; (1S,3s)-1-methyl-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile (6.2 mg, 29.5%). LCMS m/z=403.3 [M+H]⁺; ¹H NMR (400 MHz, MeOH-d₄) δ: 8.20 (s, 1H), 8.06 (s, 1H), 7.86 (d, 1H), 6.45 (d, 1H), 4.62-4.55 (m, 2H), 3.98 (s, 3H), 3.36-3.29 (m, 2H), 3.26-3.23 (m, 3H), 2.48-2.47 (m, 2H), 2.45-2.36 (m, 2H), 1.99-1.97 (m, 2H), 1.89-1.87 (m, 2H), 1.56 (s, 3H).

### Example 3 and 4

### (1R,3r)-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile and (1S,3s)-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile

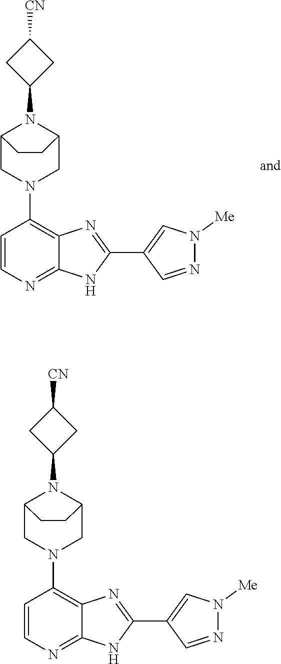 [SMILES: Cn1cc(-c2nc3c(N4CC5CCC(C4)N5[C@H]4C[C@H](C#N)C4)ccnc3[nH]2)cn1 ; Cn1cc(-c2nc3c(N4CC5CCC(C4)N5[C@H]4C[C@@H](C#N)C4)ccnc3[nH]2)cn1]

* Relative stereochemistry arbitrarily assigned

The title compounds were prepared from 7-(3,8-diazabicyclo[3.2.1]octan-3-yl)-2-(1-methyl-1H-pyrazol-4-yl)-3H-imidazo[4,5-b]pyridine hydrochloride (Preparation 12, 80 mg, 0.231 mmol) and 3-oxocyclobutane-1-carbonitrile using an analogous method to that described for Example 1 and 2.

*Peak 1, Example 3; (1R,3r)-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile (10.6 mg, 26.5%). LCMS m/z=389.2 [M+H]⁺; ¹H NMR (400 MHz, DMSO-d₆) δ: 12.89 (brs, 1H), 8.26 (s, 1H), 7.99 (s, 1H), 7.86-7.85 (m, 1H), 6.37-6.35 (m, 1H), 4.51-4.49 (m, 2H), 3.91 (s, 3H), 3.32-3.08 (m, 7H), 2.40-2.25 (m, 4H), 1.87-1.69 (m, 3H).

*Peak 2, Example 4; (1S,3s)-3-(3-(2-(1-methyl-1H-pyrazol-4-yl)-3H-imidazo[4,5-b]pyridin-7-yl)-3,8-diazabicyclo[3.2.1]octan-8-yl)cyclobutane-1-carbonitrile; (22.8 mg, 57%). LCMS m/z=389.2 [M+H]⁺; ¹H NMR (400 MHz, MeOH-d₄) δ: 8.20 (s, 1H), 8.05 (s, 1H), 7.85 (d, 1H), 6.45 (d, 1H), 4.57-4.54 (m 2H), 3.98 (s, 3H), 3.38-3.35 (m, 2H), 3.25-3.18 (m, 3H), 3.16-3.00 (m, 1H), 2.64-2.60 (m, 2H), 2.27-2.25 (m, 2H), 1.98-1.87 (m, 4H).

### Example 5 and 6

### 7-(8-((1r,3R)-3-methoxycyclobutyl)-3,8-diazabicyclo[3.2.1]octan-3-yl)-2-(1-methyl-1H-pyrazol-4-yl)-3H-imidazo[4,5-b]pyridine and 7-(8-((1s,3S)-3-methoxycyclobutyl)-3,8-diazabicyclo[3.2.1]octan-3-yl)-2-(1-methyl-1H-pyrazol-4-yl)-3H-imidazo[4,5-b]pyridine

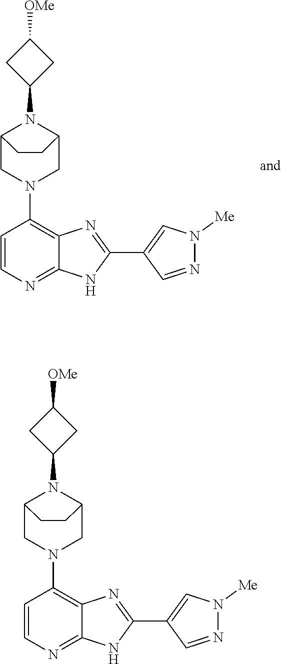 [SMILES: CO[C@H]1C[C@H](N2C3CCC2CN(c2ccnc4[nH]c(-c5cnn(C)c5)nc24)C3)C1 ; CO[C@H]1C[C@@H](N2C3CCC2CN(c2ccnc4[nH]c(-c5cnn(C)c5)nc24)C3)C1]

* Relative stereochemistry arbitrarily assigned

The title compounds were prepared from 7-(3,8-diazabicyclo[3.2.1]octan-3-yl)-2-(1-methyl-1H-pyrazol-4-yl)-3H-imidazo[4,5-b]pyridine hydrochloride (Preparation 12, 50 mg, 0.162 mmol) and 3-methoxycyclobutan-1-one using an analogous method to that described for Example 1 and 2.

*Peak 1, Example 5; 7-(8-((1r,3R)-3-methoxycyclobutyl)-3,8-diazabicyclo[3.2.1]octan-3-yl)-2-(1-methyl-1H-pyrazol-4-yl)-3H-imidazo[4,5-b]pyridine (9.3 mg, 27.8%). LCMS m/z=394.3 [M+H]⁺; ¹H NMR (500 MHz, CDCl₃) δ: 8.08 (s, 1H), 8.01 (s, 2H), 6.34 (d, 1H), 4.60-4.58 (m, 2H), 4.09-4.06 (m, 1H), 3.99 (s, 3H), 3.41-3.34 (m, 4H), 3.27 (s, 3H), 3.23-3.21 (m, 1H), 2.22-2.14 (m, 4H), 1.95-1.86 (m, 4H)

*Peak 2, Example 6; 7-(8-((1s,3S)-3-methoxycyclobutyl)-3,8-diazabicyclo[3.2.1]octan-3-yl)-2-(1-methyl-1H-pyrazol-4-yl)-3H-imidazo[4,5-b]pyridine (19.8 mg, 59.6%). LCMS m/z=389.2 [M+H]⁺; ¹H NMR (400 MHz, CDCl₃) δ: 8.07 (s, 1H), 8.02 (s, 1H), 8.00-7.98 (m, 1H), 6.33 (d, 1H), 4.58-4.56 (m, 2H), 3.99 (s, 3H), 3.65-3.62 (m, 1H), 3.39-3.34 (m, 4H), 3.25 (s, 3H), 2.53-2.51 (m, 1H), 2.50-2.49 (m, 2H), 1.95-1.85 (m, 6H).

### Example 7 and 8

### (1RS,2RS)-2-(3-(2-(1-methyl-1H-pyrazol-4-yl)-3H-imidazo[4,5-b]pyridin-7-yl)-3,8-diazabicyclo[3.2.1]octan-8-yl)cyclobutan-1-ol and (1RS,2SR)-2-(3-(2-(1-methyl-1H-pyrazol-4-yl)-3H-imidazo[4,5-b]pyridin-7-yl)-3,8-diazabicyclo[3.2.1]octan-8-yl)cyclobutan-1-ol

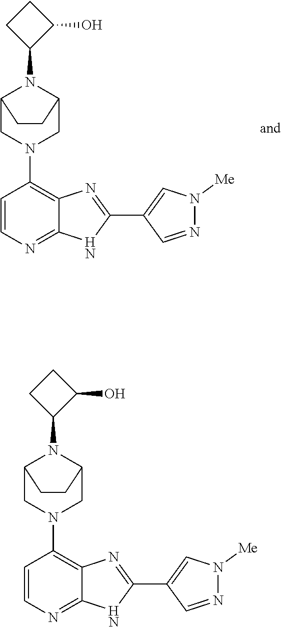 [SMILES: Cn1cc(-c2nc3c(N4CC5CCC(C4)N5[C@H]4CC[C@@H]4O)ccnc3[nH]2)cn1 ; Cn1cc(-c2nc3c(N4CC5CCC(C4)N5[C@H]4CC[C@H]4O)ccnc3[nH]2)cn1]

* Relative stereochemistry arbitrarily assigned

Part 1. NaBH₃CN (60.9 mg, 0.970 mmol) was added to a solution of 7-(3,8-diazabicyclo[3.2.1]octan-3-yl)-2-(1-methyl-1H-pyrazol-4-yl)-3H-imidazo[4,5-b]pyridine hydrochloride (Preparation 12, 50 mg, 0.145 mmol) and 2-((tert-butyldimethylsilyl)oxy)cyclobutan-1-one (323.8 mg, 1.62 mmol) in MeOH (5 mL) and the resulting mixture stirred at 50° C. for 18 h. The reaction was diluted with H₂O (20 mL) and extracted with EtOAc (3×20 mL). The combined organics were washed with brine (15 mL), dried (Na₂SO₄) and evaporated to dryness in vacuo. The residue was purified by prep-TLC (DCM/MeOH, 10:1) to afford 7-((1R,5S)-8-(2-((tert-butyldimethylsilyl)oxy)cyclobutyl)-3,8-diazabicyclo[3.2.1]octan-3-yl)-2-(1-methyl-1H-pyrazol-4-yl)-3H-imidazo[4,5-b]pyridine as a yellow oil (130 mg, 81.5%). LCMS m/z=494.4 [M+H]⁺.

Part 2. HCl/dioxane (4 M, 5 mL) was added to a solution of 7-((1R,5S)-8-(2-((tert-butyldimethylsilyl)oxy)cyclobutyl)-3,8-diazabicyclo[3.2.1]octan-3-yl)-2-(1-methyl-1H-pyrazol-4-yl)-3H-imidazo[4,5-b]pyridine (Part 1, 130 mg, 0.263 mmol) in DCM (6 mL). The reaction mixture was evaporated to dryness in vacuo and the residue purified by prep-HPLC-4 (Gradient 15-45%) to give the isomeric title compounds.

*Peak 1, Example 7; (1RS,2RS)-2-(3-(2-(1-methyl-1H-pyrazol-4-yl)-3H-imidazo[4,5-b]pyridin-7-yl)-3,8-diazabicyclo[3.2.1]octan-8-yl)cyclobutan-1-ol as a yellow solid (29.5 mg, 29.5%). LCMS m/z=380.0 [M+H]⁺; ¹H NMR (500 MHz, DMSO-d₆) δ: 12.84 (br, 1H), 8.26 (s, 1H), 8.00 (s, 1H), 7.85 (d, 1H), 6.33 (d, 1H), 5.19-5.17 (m, 1H), 4.47-4.44 (m, 2H), 3.91 (s, 3H), 3.79-3.76 (m, 1H), 3.55-3.53 (m, 1H), 3.26-3.24 (m, 1H), 3.10-3.07 (m, 2H), 2.76-2.74 (m, 1H), 2.00-1.92 (m, 3H), 1.79-1.76 (m, 1H), 1.68-1.66 (m, 2H), 1.42-1.40 (m, 1H), 1.15-1.14 (m, 1H).

*Peak 2, Example 8; (1RS,2SR)-2-(3-(2-(1-methyl-1H-pyrazol-4-yl)-3H-imidazo[4,5-b]pyridin-7-yl)-3,8-diazabicyclo[3.2.1]octan-8-yl)cyclobutan-1-ol as a yellow solid (14.6 mg, 14.6%). LCMS m/z=380.0 [M+H]⁺; ¹H NMR (500 MHz, DMSO-d₆) δ: 12.85 (br, 1H), 8.27 (s, 1H), 8.00 (s, 1H), 7.85 (d, 1H), 6.35 (d, 1H), 4.62-4.61 (m, 1H), 4.53-4.50 (m, 2H), 4.10-4.08 (m, 1H), 3.91 (s, 3H), 3.48-3.46 (m, 1H), 3.26-3.24 (m, 1H), 3.20-3.14 (m, 2H), 3.06-3.04 (m, 1H), 2.00-1.67 (m, 6H).

### Example 9

### 2-(1-methyl-1H-pyrazol-4-yl)-7-(8-(oxetan-3-yl)-3,8-diazabicyclo[3.2.1]octan-3-yl)-3H-imidazo[4,5-b]pyridine

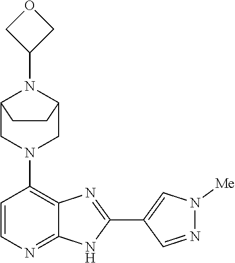 [SMILES: Cn1cc(-c2nc3c(N4CC5CCC(C4)N5C4COC4)ccnc3[nH]2)cn1]

NaBH₃CN (32.7 mg, 0.521 mmol) was added to a solution of 7-(3,8-diazabicyclo[3.2.1]octan-3-yl)-2-(1-methyl-1H-pyrazol-4-yl)-3H-imidazo[4,5-b]pyridine hydrochloride (Preparation 12, 30 mg, 0.087 mmol) and oxetan-3-one (62.5 mg, 0.868 mmol) in MeOH (5 mL) and the resulting mixture stirred at 50° C. for 16 h. The mixture was concentrated and the residue was purified by prep-HPLC-3 (gradient 7-37%) to give 2-(1-methyl-1H-pyrazol-4-yl)-7-(8-(oxetan-3-yl)-3,8-diazabicyclo[3.2.1]octan-3-yl)-3H-imidazo[4,5-b]pyridine as a white solid (5 mg, 15.8%). LCMS m/z=366.2 [M+H]⁺; ¹H NMR (400 MHz, MeOH-d₄) δ: 8.18 (s, 1H), 8.03 (s, 1H), 7.84 (d, 1H), 6.44 (d, 1H), 4.78-4.75 (m, 4H), 4.61-4.59 (m, 4H), 3.96 (s, 3H), 3.81-3.78 (m, 1H), 3.28-3.26 (m, 2H), 1.97-1.89 (m, 4H).

### Example 10

### 7-((1R,5S)-8-(3-(difluoromethyl)cyclobutyl)-3,8-diazabicyclo[3.2.1]octan-3-yl)-2-(1-methyl-1H-pyrazol-4-yl)-3H-imidazo[4,5-b]pyridine

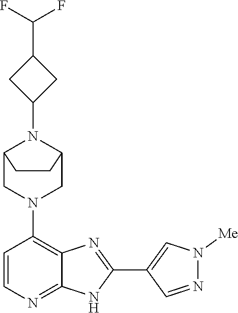 [SMILES: Cn1cc(-c2nc3c(N4CC5CCC(C4)N5C4CC(C(F)F)C4)ccnc3[nH]2)cn1]

The title compound was prepared as a yellow solid (39, 32.6%) from 7-(3,8-diazabicyclo[3.2.1]octan-3-yl)-2-(1-methyl-1H-pyrazol-4-yl)-3H-imidazo[4,5-b]pyridine hydrochloride (Preparation 12, 100 mg, 0.289 mmol) and 3-(difluoromethyl)cyclobutan-1-one using an analogous method to that described for Example 9. LCMS m/z=414.3 [M+H]⁺; ¹H NMR (500 MHz, DMSO-d₆) δ: 12.86 (br, 1H), 8.26 (s, 1H), 8.00 (s, 1H), 7.85 (d, 1H), 6.33 (d, 1H), 6.19-5.94 (m, 1H), 4.21-4.19 (m, 2H), 3.92 (s, 3H), 3.09-2.98 (m, 3H), 2.43-2.36 (m, 2H), 2.17-2.01 (m, 3H), 1.86-1.68 (m, 6H).

### Example 11

### 7-(8-(azetidin-3-yl)-3,8-diazabicyclo[3.2.1]octan-3-yl)-2-(1-methyl-1H-pyrazol-4-yl)-3H-imidazo[4,5-b]pyridine hydrochloride

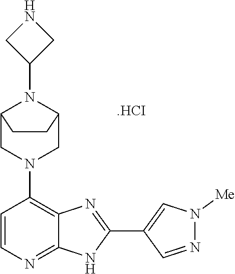

Part 1. NaBH₃CN (32.7 mg, 0.521 mmol) was added to a solution of 7-(3,8-diazabicyclo[3.2.1]octan-3-yl)-2-(1-methyl-1H-pyrazol-4-yl)-3H-imidazo[4,5-b]pyridine hydrochloride (Preparation 12, 100 mg, 0.289 mmol) and tert-butyl 3-oxoazetidine-1-carboxylate (742.5 mg, 4.34 mmol) in MeOH (6 mL) and the resulting mixture stirred at 50° C. for 36 h. The reaction mixture was concentrated and the residue diluted with H₂O (10 mL) and extracted with EtOAc (3×5 mL). The combined extracts were washed with brine (15 mL), dried (Na₂SO₄) and evaporated to dryness in vacuo. The residue was purified by column chromatography over SiO2 (DCM/MeOH, 6:1 to 0:1) to afford tert-butyl 3-(3-(2-(1-methyl-1H-pyrazol-4-yl)-3H-imidazo[4,5-b]pyridin-7-yl)-3,8-diazabicyclo[3.2.1]octan-8-yl)azetidine-1-carboxylate as a yellow oil (261.6 mg). LCMS m/z=465.2 [M+H]⁺

Part 2. HCl/dioxane (4 M, 3 mL) was added to a solution of tert-butyl 3-(3-(2-(1-methyl-1H-pyrazol-4-yl)-3H-imidazo[4,5-b]pyridin-7-yl)-3,8-diazabicyclo[3.2.1]octan-8-yl)azetidine-1-carboxylate (261.6 mg, 0.564 mmol) and the resulting mixture stirred at 25° C. for 1 h. The reaction mixture was evaporated to dryness and the residue was purified by prep-HPLC-2 (gradient 5-35%) to give 7-(8-(azetidin-3-yl)-3,8-diazabicyclo[3.2.1]octan-3-yl)-2-(1-methyl-1H-pyrazol-4-yl)-3H-imidazo[4,5-b]pyridine as a white solid (16 mg, 45.4% yield). LCMS m/z=365.2 [M+H]⁺; ¹H NMR (400 MHz, MeOH-d₄) δ: 8.19 (s, 1H), 8.05 (s, 1H), 7.87 (d, 1H), 6.46 (d, 1H), 4.58-4.55 (m, 2H), 3.98 (s, 3H), 3.98-3.86 (m, 2H), 3.79-3.74 (m, 2H), 3.60-3.58 (m, 1H), 3.40-3.36 (m, 2H), 3.28-3.24 (m, 2H), 1.99-1.90 (m, 4H).

### Example 12

### 2-(1-methyl-1H-pyrazol-4-yl)-7-(8-(1-methylazetidin-3-yl)-3,8-diazabicyclo[3.2.1]octan-3-yl)-3H-imidazo[4,5-b]pyridine trifluoroacetate

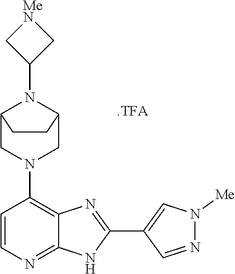 [SMILES: CN1CC(N2C3CCC2CN(c2ccnc4[nH]c(-c5cnn(C)c5)nc24)C3)C1 ; ".TFA"]

To a mixture of 7-(8-(azetidin-3-yl)-3,8-diazabicyclo[3.2.1]octan-3-yl)-2-(1-methyl-1H-pyrazol-4-yl)-3H-imidazo[4,5-b]pyridine hydrochloride (Example 11, 70.6 mg, 0.176 mmol) in MeOH (3 mL) was added paraformaldehyde (39.6 mg, 0.440 mmol) and NaBH₃CN (33.2 mg, 0.528 mmol) and the mixture stirred at 25° C. for 16 h. The reaction mixture was concentrated and the residue was purified by prep-HPLC-5 (gradient 10-30%) to give 2-(1-methyl-1H-pyrazol-4-yl)-7-(8-(1-methylazetidin-3-yl)-3,8-diazabicyclo[3.2.1]octan-3-yl)-3H-imidazo[4,5-b]pyridine as a white solid (16 mg, 45.4%). LCMS m/z=379.2 [M+H]⁺; ¹H NMR (400 MHz, MeOH-d₄) δ: 8.36 (s, 1H), 8.13 (s, 1H), 7.92 (d, 1H), 6.77 (d, 1H), 4.60-4.55 (m, 2H), 4.43-4.25 (m, 2H), 4.11-4.07 (m, 2H), 3.99 (s, 3H), 3.62-3.51 (m, 5H), 3.06-2.99 (m, 3H), 2.01-1.99 (m, 2H), 1.89-1.81 (m, 2H).

### Example 13

### 3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile

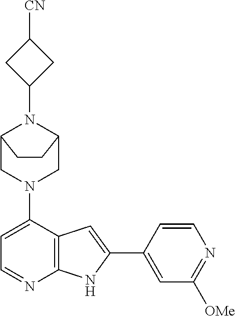 [SMILES: COc1cc(-c2cc3c(N4CC5CCC(C4)N5C4CC(C#N)C4)ccnc3[nH]2)ccn1]

NaBH₃CN (67.6 mg, 1.08 mmol) was added to a solution of 4-(3,8-diazabicyclo[3.2.1]octan-3-yl)-2-(2-methoxypyridin-4-yl)-1H-pyrrolo[2,3-b]pyridine hydrochloride (Preparation 22, 80 mg, 0.215 mmol) and 3-oxocyclobutane-1-carbonitrile (102.3 mg, 1.08 mmol) in MeOH (5 mL) and the resulting mixture stirred at 50° C. for 18 h. The reaction mixture was concentrated and purified by prep-HPLC-3 (gradient 27-57%) to give 3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile as a yellow solid (44.9 mg, 50.4%). LCMS m/z=415.3 [M+H]⁺; ¹H NMR (400 MHz, CDCl₃) δ: 8.21 (d, 1H), 8.11 (d, 1H), 7.29-7.27 (m, 1H), 7.16 (s, 1H), 6.94 (s, 1H), 6.37 (d, 1H), 4.01 (s, 3H), 3.84-3.79 (m, 2H), 3.37-3.34 (m, 4H), 3.08-3.06 (m, 1H), 2.81-2.60 (m, 1H), 2.58-2.51 (m, 2H), 2.37-2.33 (m, 2H), 1.95-1.87 (m, 4H).

### Example 14 and 15

### (1S,3s)-3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile and (1R,3r)-3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile

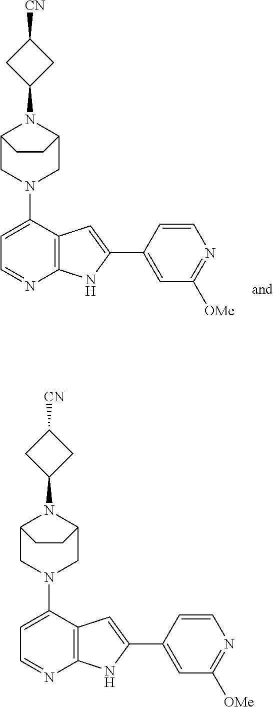 [SMILES: COc1cc(-c2cc3c(N4CC5CCC(C4)N5[C@H]4C[C@@H](C#N)C4)ccnc3[nH]2)ccn1 ; COc1cc(-c2cc3c(N4CC5CCC(C4)N5[C@H]4C[C@H](C#N)C4)ccnc3[nH]2)ccn1]

* Relative stereochemistry arbitrarily assigned

The title compounds were obtained from 3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile (Example 13) by SFC chromatography (SFC-2).

*Peak 1, Example 14. (1S,3s)-3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile; LCMS m/z=415.3 [M+H]⁺; ¹H NMR (500 MHz, CDCl₃) δ: 8.15 (d, 1H), 7.92 (br d, 1H), 7.14 (dd, 1H), 7.00 (s, 1H), 6.88 (s, 1H), 6.30 (d, 1H), 3.80 (br d, 2H), 3.41-3.25 (m, 5H), 3.16-3.09 (m, 1H), 2.45 (ddd, 2H), 2.34-2.22 (m, 2H), 1.99-1.90 (m, 3H), 1.79 (br d, 4H).

*Peak 2, Example 15. (1R,3r)-3-(3-(2-(2-methoxypyridin-4-yl)-1H-pyrrolo[2,3-b]pyridin-4-yl)-3,8-diazabicyclo[3.2.1]octan-8-yl)cyclobutane-1-carbonitrile; LCMS m/z=415.3 [M+H]⁺; ¹H NMR (500 MHz, CDCl₃) δ: 8.24 (d, 1H), 8.10 (d, 1H), 7.23 (dd, 1H), 7.09 (s, 1H), 6.96 (s, 1H), 6.37 (d, 1H), 4.03 (s, 3H), 3.81 (br d, 2H), 3.40-3.32 (m, 4H), 3.10 (s, 1H), 2.84 (s, 1H), 2.65-2.51 (m, 2H), 2.43-2.30 (m, 2H), 1.91 (br s, 4H).

### Example 16 and 17

### (1S,3s)-3-(3-(2-(1-methyl-1H-pyrazol-4-yl)-1H-pyrrolo[2,3-b]pyridin-4-yl)-3,8-diazabicyclo[3.2.1]octan-8-yl)cyclobutane-1-carbonitrile and (1R,3r)-3-(3-(2-(1-methyl-1H-pyrazol-4-yl)-1H-pyrrolo[2,3-b]pyridin-4-yl)-3,8-diazabicyclo[3.2.1]octan-8-yl)cyclobutane-1-carbonitrile

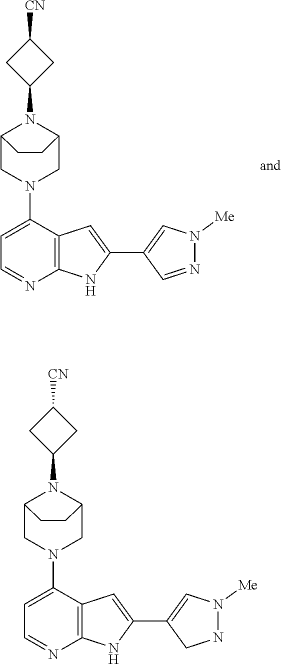 [SMILES: Cn1cc(-c2cc3c(N4CC5CCC(C4)N5[C@H]4C[C@@H](C#N)C4)ccnc3[nH]2)cn1 ; CN1C=C(c2cc3c(N4CC5CCC(C4)N5[C@H]4C[C@H](C#N)C4)ccnc3[nH]2)CN1]

* Relative stereochemistry arbitrarily assigned

Part 1. NaBH₃CN (145.8 mg, 2.32 mmol) was added to a solution of 4-(3,8-diazabicyclo[3.2.1]octan-3-yl)-2-(1-methyl-1H-pyrazol-4-yl)-1H-pyrrolo[2,3-b]pyridine hydrochloride (Preparation 20 Sch-26-01, 160 mg, 0.519 mmol) and 3-oxocyclobutane-1-carbonitrile (353 mg, 3.71 mmol) in MeOH (6 mL) and the resulting mixture stirred at 50° C. for 40 h. The reaction mixture was concentrated and purified by prep-HPLC-4 (gradient 21-51% to give 3-(3-(2-(1-methyl-1H-pyrazol-4-yl)-1H-pyrrolo[2,3-b]pyridin-4-yl)-3,8-diazabicyclo[3.2.1]octan-8-yl)cyclobutane-1-carbonitrile as a white solid (85 mg, 47.3%). LCMS m/z=388.1 [M+H]⁺

Part 2. The title compounds were obtained from 3-(3-(2-(1-methyl-1H-pyrazol-4-yl)-1H-pyrrolo[2,3-b]pyridin-4-yl)-3,8-diazabicyclo[3.2.1]octan-8-yl)cyclobutane-1-carbonitrile (Part 1) by SFC chromatography (SFC-1, 35% EtOH).

*Peak 1, Example 16. (1S,3s)-3-(3-(2-(1-methyl-1H-pyrazol-4-yl)-1H-pyrrolo[2,3-b]pyridin-4-yl)-3,8-diazabicyclo[3.2.1]octan-8-yl)cyclobutane-1-carbonitrile (42.3 mg). LCMS m/z=388.3 [M+H]⁺; ¹H NMR (400 MHz, MeOH-d₄) δ: 7.99 (s, 1H), 7.89 (s, 1H), 7.82 (d, 1H), 6.67 (s, 1H), 6.38 (d, 1H), 3.94 (s, 3H), 3.86-3.83 (m, 2H), 3.45-3.40 (m, 3H), 3.25-3.21 (m, 3H), 2.49-2.37 (m, 4H), 2.01-1.87 (m, 4H).

*Peak 2, Example 17. (1S,3s)-3-(3-(2-(1-methyl-1H-pyrazol-4-yl)-1H-pyrrolo[2,3-b]pyridin-4-yl)-3,8-diazabicyclo[3.2.1]octan-8-yl)cyclobutane-1-carbonitrile (17.8 mg). LCMS m/z=388.3 [M+H]⁺; ¹H NMR (400 MHz, MeOH-d₄) δ: 7.99 (s, 1H), 7.89 (s, 1H), 7.83 (d, 1H), 6.68 (s, 1H), 6.40 (d, 1H), 3.94 (s, 3H), 3.86-3.83 (m, 2H), 3.41-3.28 (m, 2H), 3.17-3.25 (m, 3H), 3.00-2.98 (m, 1H), 2.65-2.58 (m, 2H), 2.27-2.25 (m, 2H), 2.01-1.89- (m, 4H).

### Example 18

### 2-(2-methoxypyridin-4-yl)-4-(8-(oxetan-3-yl)-3,8-diazabicyclo[3.2.1]octan-3-yl)-1H-pyrrolo[2,3-b]pyridine

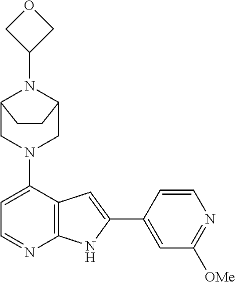 [SMILES: COc1cc(-c2cc3c(N4CC5CCC(C4)N5C4COC4)ccnc3[nH]2)ccn1]

Part 1. NaBH₃CN (67.6 mg, 1.08 mmol) was added to a solution of 4-(3,8-diazabicyclo[3.2.1]octan-3-yl)-2-(2-methoxypyridin-4-yl)-1-tosyl-1H-pyrrolo[2,3-b]pyridine hydrochloride (Preparation 21, 80 mg, 0.215 mmol) and oxetan-3-one (58.9 mg, 0.817 mmol) in MeOH (3 mL) and the resulting mixture stirred at 50° C. for 18 h. The reaction mixture was diluted with H₂O (10 mL) and extracted with EtOAc (3×20 mL). The combined organics were dried (Na₂SO₄) and evaporated to dryness in vacuo. The residue was purified by prep-TLC (10:1 DCM/MeOH) to give 2-(2-methoxypyridin-4-yl)-4-(8-(oxetan-3-yl)-3,8-diazabicyclo[3.2.1]octan-3-yl)-1-tosyl-1H-pyrrolo[2,3-b]pyridine as a white solid (30 mg) which was used without further purification. ¹H NMR (400 MHz, CDCl₃) δ: 8.21-8.16 (m, 2H), 7.84-7.82 (m, 2H), 7.20-7.18 (m, 2H), 7.09-7.08 (m, 1H), 6.89 (s, 1H), 6.60 (s, 1H), 6.43 (d, 1H), 4.75-4.73 (m, 2H), 4.58-4.55 (m, 2H), 4.01 (s, 3H), 3.70-3.65 (m, 1H), 3.64-3.56 (m, 2H), 3.33-3.32 (m, 2H), 3.30-3.20 (m, 2H), 2.35 (s, 3H), 1.89-1.74 (m, 4H).

Part 2. 5 N NaOH (4.4 mg, 0.110 mmol) was added to a solution of 2-(2-methoxypyridin-4-yl)-4-(8-(oxetan-3-yl)-3,8-diazabicyclo[3.2.1]octan-3-yl)-1-tosyl-1H-pyrrolo[2,3-b]pyridine (Part 1, 30.0 mg, 0.055 mmol) in MeOH (2 mL) and the resulting mixture stirred at 50° C. for 8 h. The reaction mixture was concentrated and purified by prep-HPLC-3 (gradient 23-52%) to give 2-(2-methoxypyridin-4-yl)-4-(8-(oxetan-3-yl)-3,8-diazabicyclo[3.2.1]octan-3-yl)-1H-pyrrolo[2,3-b]pyridine as a yellow solid (10 mg, 11.9% over 2 parts). LCMS m/z=392.2 [M+H]⁺; ¹H NMR (500 MHz, CDCl₃) δ: 8.21 (d, 1H), 8.08 (d, 1H), 7.24-7.23 (m, 1H), 7.10 (s, 1H), 6.95 (s, 1H), 6.37 (d, 1H), 4.77-4.75 (m, 2H), 4.63-4.61 (m, 2H), 4.00 (s, 3H), 3.86-3.83 (m, 2H), 3.73-3.77 (m, 1H), 3.45-3.47 (m, 2H), 3.29-3.40 (m, 2H), 1.87-1.96 (m, 4H).

### Example 19

### 4-(8-(3-(difluoromethyl)cyclobutyl)-3,8-diazabicyclo[3.2.1]octan-3-yl)-2-(2-methoxypyridin-4-yl)-1H-pyrrolo[2,3-b]pyridine

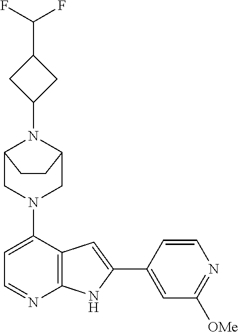 [SMILES: COc1cc(-c2cc3c(N4CC5CCC(C4)N5C4CC(C(F)F)C4)ccnc3[nH]2)ccn1]

NaBH₃CN (46.2 mg, 0.735 mmol) was added to a solution of 4-(3,8-diazabicyclo[3.2.1]octan-3-yl)-2-(2-methoxypyridin-4-yl)-1-tosyl-1H-pyrrolo[2,3-b]pyridine hydrochloride (Preparation 21, 60 mg, 0.123 mmol) and 3-(difluoromethyl)cyclobutan-1-one (73.6 mg, 0.613 mmol) in MeOH (5 mL) and the resulting mixture stirred at 50° C. for 16 h. The mixture was purified by prep-HPLC-3 (gradient 39-69%) to give 4-(8-(3-(difluoromethyl)cyclobutyl)-3,8-diazabicyclo[3.2.1]octan-3-yl)-2-(2-methoxypyridin-4-yl)-1H-pyrrolo[2,3-b]pyridine as a white solid (5.5 mg, 10.2%). LCMS m/z=440.3 [M+H]⁺; ¹H NMR (400 MHz, DMSO-d₃) δ: 11.98 (brs, 1H), 8.11 (d, 1H), 7.88 (d, 1H), 7.50 (d, 1H), 7.33 (d, 2H), 6.27 (d, 1H), 6.17-5.88 (m, 1H), 4.08-4.06 (m, 1H), 3.84 (s, 3H), 3.77-3.74 (m, 2H), 3.14-3.10 (m, 3H), 3.01-2.96 (m, 1H), 2.15-2.11 (m, 1H), 1.84-1.83 (m, 2H), 1.77-1.67 (m, 6H)

### Example 20

### 2-(3-(3-(6-(1-methyl-1H-pyrazol-4-yl)pyrrolo[1,2-b]pyridazin-4-yl)-3,8-diazabicyclo[3.2.1]octan-8-yl)oxetan-3-yl)acetonitrile

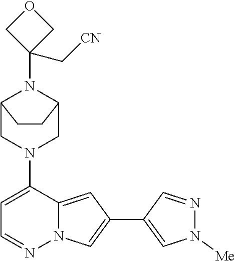 [SMILES: Cn1cc(-c2cc3c(N4CC5CCC(C4)N5C4(CC#N)COC4)ccnn3c2)cn1]

A mixture of 4-(3,8-diazabicyclo[3.2.1]octan-3-yl)-6-(1-methyl-1H-pyrazol-4-yl)pyrrolo[1,2-b]pyridazine hydrochloride (Preparation 29, 40 mg, 0.116 mmol), 2-(oxetan-3-ylidene)acetonitrile (110 mg, 1.16 mmol) and TEA (117 mg, 1.16 mmol) in DMF (1 mL) heated in a sealed vial at 100° C. for 24 h. The reaction mixture was cooled to rt, diluted with EtOAc and sat. aq NH₄Cl and filtered through celite. The layers were separated and the aqueous extracted with EtOAc (2×). The combined organics were washed with brine, dried (MgSO₄) and evaporated to dryness in vacuo. The residue was dissolved in DMSO (2 mL) and purified by PSR auto-purification to afford 2-(3-(3-(6-(1-methyl-1H-pyrazol-4-yl)pyrrolo[1,2-b]pyridazin-4-yl)-3,8-diazabicyclo[3.2.1]octan-8-yl)oxetan-3-yl)acetonitrile (6.6 mg, 14.1%). LCMS m/z=404.2 [M+H]⁺.

### Example 21

### 3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile

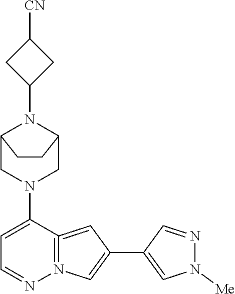

NaBH₃CN (43.7 mg, 0.696 mmol) was added to a solution of 4-(3,8-diazabicyclo[3.2.1]octan-3-yl)-6-(1-methyl-1H-pyrazol-4-yl)pyrrolo[1,2-b]pyridazine hydrochloride (Preparation 29, 80 mg, 0.232 mmol) and 3-oxocyclobutanecarbonitrile (110.3 mg, 1.16 mmol) in MeOH (2.3 mL) and the mixture stirred at 50° C. overnight. The reaction mixture was evaporated to dryness in vacuo and the residue diluted with sat. aq NaHCO₃, H₂O and EtOAc. The organic layer was separated and the aqueous layer extracted with EtOAc (×2). The combined organics were dried (MgSO₄) and evaporated to dryness in vacuo and purified by PSR auto-purification to afford 3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile. LCMS m/z=388.2 [M+H]⁺.

### Example 22-26

The title compounds were prepared from 4-(3,8-diazabicyclo[3.2.1]octan-3-yl)-6-(1-methyl-1H-pyrazol-4-yl)pyrrolo[1,2-b]pyridazine hydrochloride (Preparation 29) and the appropriate ketone using an analogous method to that described for Example 21.

| Example No. | Name/Structure/HPLC/Reactants/Data |
|---|---|
| 22 | 3-(3-(6-(1-methyl-1H-pyrazol-4-yl)pyrrolo[1,2-b]pyridazin-4-yl)-3,8- |
| | diazabicyclo[3.2.1]octan-8-yl)-1-(trifluoromethyl)cyclobutan-1-ol |
| | 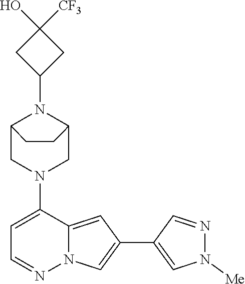 [SMILES: Cn1cc(-c2cc3c(N4CC5CCC(C4)N5C4CC(O)(C(F)(F)F)C4)ccnn3c2)cn1] |
| | Ketone: 3-hydroxy-3-(trifluoromethyl)cyclobutan-1-one |
| | Yield: 17.1 mg, 22%; LCMS m/z = 447 [M + H]⁺ |
| 23 | 6-(1-methyl-1H-pyrazol-4-yl)-4-(8-(oxetan-3-y1)-3,8- |
| | diazabicyclo[3.2.1]octan-3-yl)pyrrolo[1,2-b]pyridazine |
| | 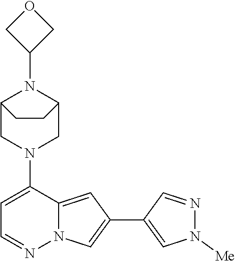 [SMILES: Cn1cc(-c2cc3c(N4CC5CCC(C4)N5C4COC4)ccnn3c2)cn1] |
| | Ketone: oxetan-3-one |
| | Yield: 18.8 mg, 30%; LCMS m/z = 365 [M + H]⁺ |
| 24 | 4-(8-(3-methoxycyclobutyl)-3,8-diazabicyclo[3.2.1]octan-3-yl)-6-(1- |
| | methyl-1H-pyrazol-4-yl)pyrrolo[1,2-b]pyridazine |
| | 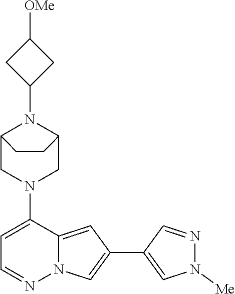 [SMILES: COC1CC(N2C3CCC2CN(c2ccnn4cc(-c5cnn(C)c5)cc24)C3)C1] |
| | Ketone: 3-methoxycyclobutan-1-one |
| | Yield: 38.8 mg, 57%; LCMS m/z = 393 [M + H]⁺ |
| 25 | 3-(3-(6-(1-methyl-1H-pyrazol-4-yl)pyrrolo[1,2-b]pyridazin-4-yl)-3,8- |
| | diazabicyclo[3.2.1]octan-8-yl)cyclobutan-1-ol |
| | 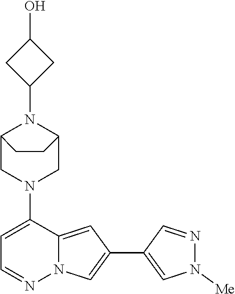 [SMILES: Cn1cc(-c2cc3c(N4CC5CCC(C4)N5C4CC(O)C4)ccnn3c2)cn1] |
| | Ketone: 3-hydroxycyclobutan-1-one |
| | Yield: 9.7 mg, 15%; LCMS m/z = 379 [M + H]⁺ |
| 26 | 4-(8-cyclobutyl-3,8-diazabicyclo[3.2.1]octan-3-y1)-6-(1-methyl-1H- |
| | pyrazol-4-yl)pyrrolo[1,2-b]pyridazine N |
| | 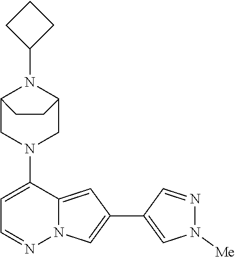 [SMILES: Cn1cc(-c2cc3c(N4CC5CCC(C4)N5C4CCC4)ccnn3c2)cn1] |
| | Ketone: cyclobutanone |
| | Yield: 19.6 mg, 31%; LCMS m/z = 363 [M + H]⁺ |

### Example 27 and 28

### (1R,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile and (1S,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile

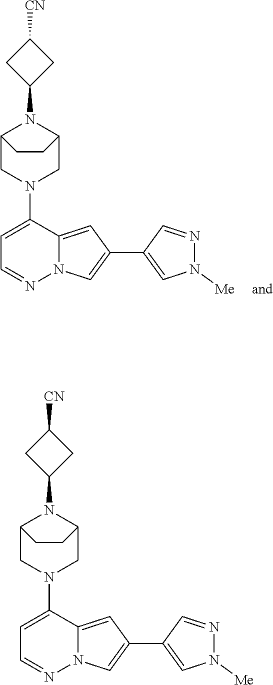 [SMILES: Cn1cc(-c2cc3c(N4CC5CCC(C4)N5[C@H]4C[C@H](C#N)C4)ccnn3c2)cn1 ; Cn1cc(-c2cc3c(N4CC5CCC(C4)N5[C@H]4C[C@@H](C#N)C4)ccnn3c2)cn1]

* Relative stereochemistry arbitrarily assigned

The title compounds were obtained from 3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile (Example 21 using SFC chromatography (SFC-3).

*Peak 1, Example 27; (1R,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile. Yield: 67.9 mg, 15.1%; LCMS m/z=388.3 [M+H]⁺; ¹H NMR (400 MHz, CDCl₃) δ: 7.76-7.70 (m, 3H), 7.56 (s, 1H), 6.51 (d, 1H), 5.71 (d, 1H), 3.94 (s, 3H), 3.72-3.69 (m, 2H), 3.37-3.30 (m, 3H), 3.18-3.15 (m, 3H), 2.49-2.46 (m, 2H), 2.34-2.30 (m, 2H), 1.94-1.92 (m, 4H).

*Peak 2, Example 28; (1S,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile. Yield: 157.3 mg, 35%; LCMS m/z=388.3 [M+H]⁺; ¹H NMR (400 MHz, CDCl₃) δ: 7.76-7.70 (m, 3H), 7.56 (s, 1H), 6.51 (s, 1H), 5.71 (d, 1H), 3.94 (s, 3H), 3.79-3.69 (m, 2H), 3.30-3.28 (m, 2H), 3.20-3.17 (m, 2H), 3.06-3.01 (m, 1H), 2.80-2.76 (m, 1H), 2.56-2.53 (m, 2H), 2.35-2.29 (m, 2H), 1.90-1.88 (m, 4H).

### Example 29 and 30

### 4-(8-((1r,3R)-3-methoxycyclobutyl)-3,8-diazabicyclo[3.2.1]octan-3-yl)-6-(1-methyl-1H-pyrazol-4-yl)pyrrolo[1,2-b]pyridazine and 4-(8-((1s,3S)-3-methoxycyclobutyl)-3,8-diazabicyclo[3.2.1]octan-3-yl)-6-(1-methyl-1H-pyrazol-4-yl)pyrrolo[1,2-b]pyridazine

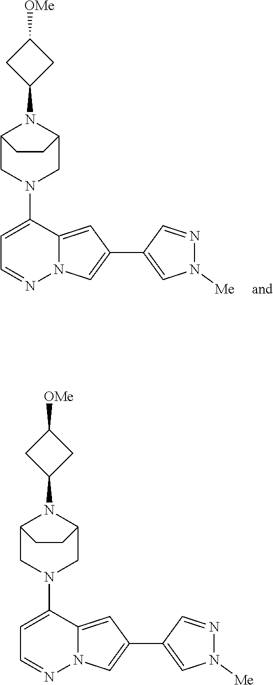 [SMILES: CO[C@H]1C[C@H](N2C3CCC2CN(c2ccnn4cc(-c5cnn(C)c5)cc24)C3)C1 ; CO[C@H]1C[C@@H](N2C3CCC2CN(c2ccnn4cc(-c5cnn(C)c5)cc24)C3)C1]

* Relative stereochemistry arbitrarily assigned

The title compounds were obtained from 4-(8-(3-methoxycyclobutyl)-3,8-diazabicyclo[3.2.1]octan-3-yl)-6-(1-methyl-1H-pyrazol-4-yl)pyrrolo[1,2-b]pyridazine (Example 24) using SFC chromatography (SFC-4).

Peak 1, Example 29; 4-(8-((1r,3R)-3-methoxycyclobutyl)-3,8-diazabicyclo[3.2.1]octan-3-yl)-6-(1-methyl-1H-pyrazol-4-yl)pyrrolo[1,2-b]pyridazine. Yield: 8.1 mg, 22.4%

Peak 2, Example 30; 4-(8-((1s,3S)-3-methoxycyclobutyl)-3,8-diazabicyclo[3.2.1]octan-3-yl)-6-(1-methyl-1H-pyrazol-4-yl)pyrrolo[1,2-b]pyridazine. Yield: 9.4 mg, 26%

### Example 31 and 32

### (1R,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol and (1S,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol

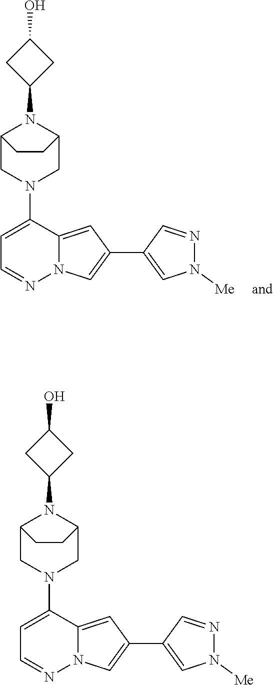 [SMILES: Cn1cc(-c2cc3c(N4CC5CCC(C4)N5[C@H]4C[C@H](O)C4)ccnn3c2)cn1 ; Cn1cc(-c2cc3c(N4CC5CCC(C4)N5[C@H]4C[C@@H](O)C4)ccnn3c2)cn1]

* Relative stereochemistry arbitrarily assigned

The title compounds were obtained from 3-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol (Example 25,) using SFC chromatography (SFC-5).

*Peak 1, Example 31; (1R,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol. Yield: 6.2 mg, 10.1%; LCMS m/z=379.3 [M+H]⁺

*Peak 2, Example 32; (1S,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol. Yield: 10.7 mg, 17.5%; LCMS m/z=379.3 [M+H]⁺

### Example 33

### 4-(8-(2-oxaspiro[3.3]heptan-6-yl)-3,8-diazabicyclo[3.2.1]octan-3-yl)-6-(1-methyl-1H-pyrazol-4-yl)pyrrolo[1,2-b]pyridazine

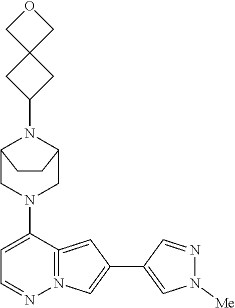

NaBH₃CN (27.3 mg, 0.435 mmol) was added to a solution of 4-(3,8-diazabicyclo[3.2.1]octan-3-yl)-6-(1-methyl-1H-pyrazol-4-yl)pyrrolo[1,2-b]pyridazine hydrochloride (Preparation 29, 50 mg, 0.232 mmol) and 2-oxaspiro[3.3]heptan-6-one (110.3 mg, 1.16 mmol) in MeOH (5 mL) and the mixture stirred at 50° C. overnight. Additional 2-oxaspiro[3.3]heptan-6-one (81.3 mg, 0.725 μmol) and NaBH₃CN (27.3 mg, 0.435 μmol) was added and the reaction stirred for 2 h at 50° C. The reaction mixture was evaporated to dryness and the residue purified by prep-HPLC-4 (gradient 23-53%) to afford 4-(8-(2-oxaspiro[3.3]heptan-6-yl)-3,8-diazabicyclo[3.2.1]octan-3-yl)-6-(1-methyl-1H-pyrazol-4-yl)pyrrolo[1,2-b]pyridazine as a yellow solid (39.5 mg, 67.4%). LCMS m/z=405.3 [M+H]⁺; H NMR (500 MHz, CDCl₃) δ: 7.74 (d, 1H), 7.69-7.72 (m, 2H), 7.55 (s, 1H), 6.52 (s, 1H), 5.71 (d, 1H), 4.70-4.73 (m, 2H), 4.64-4.66 (m, 2H), 3.94 (s, 3H), 3.67-3.71 (m, 2H), 3.32-3.34 (m, 2H), 3.18-3.21 (m, 2H), 2.83-2.86 (m, 1H), 2.41-2.45 (m, 2H), 2.10-2.12 (m, 2H), 1.86-1.96 (m, 4H).

### Example 34 and 35

### (1r,3r)-1-methyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile and (1s,3s)-1-methyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile

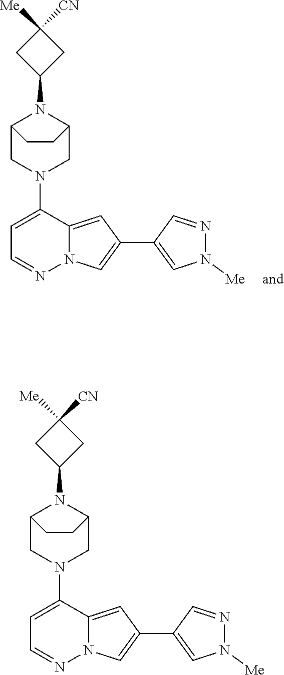 [SMILES: Cn1cc(-c2cc3c(N4CC5CCC(C4)N5[C@H]4C[C@](C)(C#N)C4)ccnn3c2)cn1 ; Cn1cc(-c2cc3c(N4CC5CCC(C4)N5[C@H]4C[C@@](C)(C#N)C4)ccnn3c2)cn1]

* Relative stereochemistry arbitrarily assigned

Part 1: NaBH₃CN (24.5 mg, 0.389 mmol) was added to a solution of 4-(3,8-diazabicyclo[3.2.1]octan-3-yl)-6-(1-methyl-1H-pyrazol-4-yl)pyrrolo[1,2-b]pyridazine hydrochloride (Preparation 29, 40 mg, 0.130 mmol) and 1-methyl-3-oxocyclobutane-1-carbonitrile (70.8 mg, 0.649 mmol) in MeOH (3 mL) and the mixture stirred at 50° C. for 18 h. The reaction mixture was evaporated to dryness and the residue purified by prep-HPLC-4 (gradient 23-53%) to afford 1-methyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile as a yellow solid (35 mg, 67.2%). LCMS m/z=402.3 [M+H]⁺.

Part 2: 1-methyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile (35.0 mg, 0.087 mmol) was purified by SFC chromatography (SFC-1, 55% IPA) to give:

*Peak 1, Example 34; (1r,3r)-1-methyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile as a yellow solid (16.5 mg, 47.1%); LCMS m/z=402.3 [M+H]⁺; ¹H NMR (500 MHz, MeOH-d₄) δ: 7.88 (s, 1H), 7.73-7.76 (m, 2H), 6.74 (d, 1H), 5.86 (d, 1H), 3.92 (s, 3H), 3.82-3.85 (m, 2H), 3.37-3.42 (m, 2H), 3.34-3.35 (m, 1H), 3.18-3.22 (m, 2H), 2.68-2.73 (m, 2H), 2.03-2.10 (m, 4H), 1.91-1.94 (m, 2H), 1.55 (s, 3H). and

*Peak 2, Example 35; (1s,3s)-1-methyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile as a yellow solid (15.6 mg, 44.6%); LCMS m/z=402.3 [M+H]⁺; ¹H NMR (500 MHz, MeOH-d₄) δ: 7.88 (s, 1H), 7.74-7.76 (m, 2H), 6.74 (s, 1H), 5.86 (d, 1H), 3.92 (s, 3H), 3.82-3.85 (m, 2H), 3.35-3.41 (m, 2H), 3.34-3.35 (m, 1H), 3.22-3.25 (m, 2H), 2.45-2.48 (m, 2H), 2.35-2.38 (m, 2H), 2.01-2.03 (m, 2H), 1.90-1.92 (m, 2H), 1.56 (s, 3H).

### Example 36 and 37

### 6-(1-methyl-1H-pyrazol-4-yl)-4-(8-((1s,3s)-3-(methylsulfonyl)cyclobutyl)-3,8-diazabicyclo[3.2.1]octan-3-yl)pyrrolo[1,2-b]pyridazine and 6-(1-methyl-1H-pyrazol-4-yl)-4-(8-((1r,3r)-3-(methylsulfonyl)cyclobutyl)-3,8-diazabicyclo[3.2.1]octan-3-yl)pyrrolo[1,2-b]pyridazine

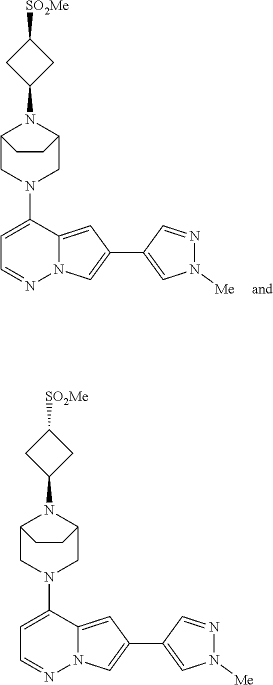 [SMILES: Cn1cc(-c2cc3c(N4CC5CCC(C4)N5[C@H]4C[C@@H](S(C)(=O)=O)C4)ccnn3c2)cn1 ; Cn1cc(-c2cc3c(N4CC5CCC(C4)N5[C@H]4C[C@H](S(C)(=O)=O)C4)ccnn3c2)cn1]

* Relative stereochemistry arbitrarily assigned

The title compounds were obtained from 4-(3,8-diazabicyclo[3.2.1]octan-3-yl)-6-(1-methyl-1H-pyrazol-4-yl)pyrrolo[1,2-b]pyridazine hydrochloride (Preparation 29) and 3-(methylsulfonyl)cyclobutan-1-one using an analogous method to that described for Example 34 and Example 35. SFC chromatography (SFC-1, 50% IPA).

*Peak 1, Example 36; 6-(1-methyl-1H-pyrazol-4-yl)-4-(8-((1s,3s)-3-(methylsulfonyl)cyclobutyl)-3,8-diazabicyclo[3.2.1]octan-3-yl)pyrrolo[1,2-b]pyridazine as a yellow solid (56 mg, 70%); LCMS m/z=441.3 [M+H]⁺; ¹H NMR (400 MHz, DMSO-d₆) δ: 8.08 (s, 1H), 7.93 (s, 1H), 7.84-7.86 (m, 2H), 6.85 (s, 1H), 5.87 (s, 1H), 3.90 (s, 3H), 3.71-3.79 (m, 3H), 3.38-3.40 (m, 2H), 3.08-3.17 (m, 3H), 2.94 (s, 3H), 2.42-2.44 (m, 2H), 2.13-2.17 (m, 2H), 1.82-1.93 (m, 4H).

*Peak 2, Example 37; 6-(1-methyl-1H-pyrazol-4-yl)-4-(8-((1r,3r)-3-(methylsulfonyl)cyclobutyl)-3,8-diazabicyclo[3.2.1]octan-3-yl)pyrrolo[1,2-b]pyridazine as a yellow solid (6 mg, 7.5%); LCMS m/z=441.3 [M+H]⁺; ¹H NMR (400 MHz, DMSO-d₆) δ: 8.00 (s, 1H), 7.86 (s, 1H), 7.76-7.78 (m, 2H), 6.78 (s, 1H), 5.81 (d, J=5.2 Hz, 1H), 3.87-3.88 (m, 1H), 3.82 (s, 3H), 3.73-3.77 (m, 2H), 3.51-3.53 (m, 2H), 3.12-3.13 (m, 3H), 2.91 (s, 3H), 2.49-2.50 (m, 2H), 2.25-2.31 (m, 2H), 1.77-1.88 (m, 4H).

### Example 38 and 39

### (1r,3r)-1-(2-methoxyethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile and (1s,3s)-1-(2-methoxyethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile

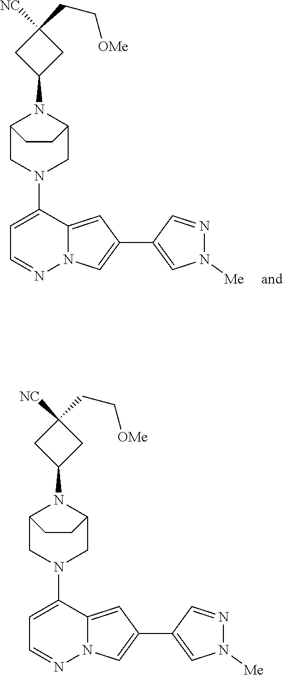 [SMILES: COCC[C@]1(C#N)C[C@H](N2C3CCC2CN(c2ccnn4cc(-c5cnn(C)c5)cc24)C3)C1 ; COCC[C@]1(C#N)C[C@@H](N2C3CCC2CN(c2ccnn4cc(-c5cnn(C)c5)cc24)C3)C1]

* Relative stereochemistry arbitrarily assigned

The title compounds were obtained 4-(3,8-diazabicyclo[3.2.1]octan-3-yl)-6-(1-methyl-1H-pyrazol-4-yl)pyrrolo[1,2-b]pyridazine hydrochloride (Preparation 29) and 1-(2-methoxyethyl)-3-oxocyclobutane-1-carbonitrile using an analogous method to that described for Example 34 and Example 35. SFC chromatography (SFC-1, 35% EtOH).

*Peak 1, Example 38; (1r,3r)-1-(2-methoxyethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile as a white solid (10.4 mg, 12%); LCMS m/z=446.2 [M+H]⁺; ¹H NMR (400 MHz, CDCl₃) δ: 7.75 (d, 1H), 7.69-7.71 (m, 2H), 7.56 (s, 1H), 6.51 (s, 1H), 5.71 (d, 1H), 3.94 (s, 3H), 3.67-3.70 (m, 2H), 3.54-3.58 (m, 2H), 3.35 (s, 3H), 3.25-3.28 (m, 2H), 3.14-3.18 (m, 2H), 2.66-2.67 (m, 2H), 1.90-2.08 (m, 9H).

*Peak 2, Example 39; (1s,3s)-1-(2-methoxyethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile as a white solid (13.6 mg, 15.7%); LCMS m/z=446.2 [M+H]⁺; ¹H NMR (400 MHz, CDCl₃) δ: 7.75 (d, 1H), 7.68-7.71 (m, 2H), 7.56 (s, 1H), 6.51 (s, 1H), 5.72 (d, 1H), 3.94 (s, 3H), 3.68-3.70 (m, 2H), 3.57-3.61 (m, 2H), 3.36 (s, 3H), 3.27-3.30 (m, 2H), 3.19-3.22 (m, 2H), 3.09-3.11 (m, 1H), 2.40-2.51 (m, 4H), 2.01-2.05 (m, 2H), 1.86-1.92 (m, 4H).

### Example 40 and 41

### 4-(8-((S)-2-oxaspiro[3.3]heptan-5-yl)-3,8-diazabicyclo[3.2.1]octan-3-yl)-6-(1-methyl-1H-pyrazol-4-yl)pyrrolo[1,2-b]pyridazine and 4-(8-((R)-2-oxaspiro[3.3]heptan-5-yl)-3,8-diazabicyclo[3.2.1]octan-3-yl)-6-(1-methyl-1H-pyrazol-4-yl)pyrrolo[1,2-b]pyridazine

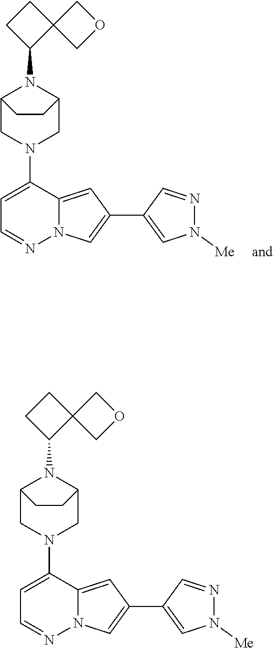 [SMILES: Cn1cc(-c2cc3c(N4CC5CCC(C4)N5[C@H]4CCC45COC5)ccnn3c2)cn1 ; Cn1cc(-c2cc3c(N4CC5CCC(C4)N5[C@@H]4CCC45COC5)ccnn3c2)cn1]

* Stereochemistry arbitrarily assigned

The title compounds were obtained 4-(3,8-diazabicyclo[3.2.1]octan-3-yl)-6-(1-methyl-1H-pyrazol-4-yl)pyrrolo[1,2-b]pyridazine hydrochloride (Preparation 29) and 2-oxaspiro[3.3]heptan-5-one using an analogous method to that described for Example 34 and Example 35. SFC chromatography (SFC-6) afforded:

*Peak 1, Example 40; 4-(8-((S)-2-oxaspiro[3.3]heptan-5-yl)-3,8-diazabicyclo[3.2.1]octan-3-yl)-6-(1-methyl-1H-pyrazol-4-yl)pyrrolo[1,2-b]pyridazine as a yellow solid (13 mg, 39.4%); LCMS m/z=405.3 [M+H]⁺; ¹H NMR (500 MHz, CDCl₃) δ: 7.77 (d, 1H), 7.71-7.73 (m, 2H), 7.58 (s, 1H), 6.56 (s, 1H), 5.74 (d, 1H), 5.30 (d, 1H), 4.62-4.66 (m, 2H), 4.47-4.50 (m, 1H), 3.95 (s, 3H), 3.73-3.80 (m, 3H), 3.27-3.39 (m, 3H), 2.97-3.00 (m, 1H), 1.86-2.13 (m, 8H).

*Peak 1, Example 41; 4-(8-((R)-2-oxaspiro[3.3]heptan-5-yl)-3,8-diazabicyclo[3.2.1]octan-3-yl)-6-(1-methyl-1H-pyrazol-4-yl)pyrrolo[1,2-b]pyridazine as a yellow solid (9 mg, 27.3%); LCMS m/z=405.3 [M+H]⁺; ¹H NMR (500 MHz, CDCl₃) δ: 7.77 (d, 1H), 7.71-7.73 (m, 2H), 7.58 (s, 1H), 6.56 (s, 1H), 5.74 (d, 1H), 5.29 (d, 1H), 4.62-4.66 (m, 2H), 4.47-4.50 (m, 1H), 3.95 (s, 3H), 3.73-3.81 (m, 3H), 3.25-3.39 (m, 3H), 2.99-3.02 (m, 1H), 1.86-2.05 (m, 8H).

### Example 42 and 43

### (1s,3s)-1-(methoxymethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile and (1r,3r)-1-(methoxymethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile

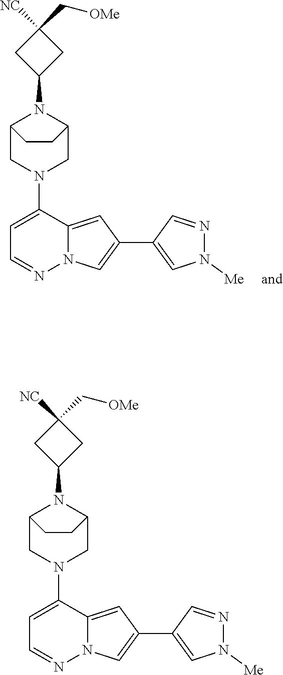 [SMILES: COC[C@]1(C#N)C[C@H](N2C3CCC2CN(c2ccnn4cc(-c5cnn(C)c5)cc24)C3)C1 ; COC[C@]1(C#N)C[C@@H](N2C3CCC2CN(c2ccnn4cc(-c5cnn(C)c5)cc24)C3)C1]

* Relative stereochemistry arbitrarily assigned

The title compounds were obtained 4-(3,8-diazabicyclo[3.2.1]octan-3-yl)-6-(1-methyl-1H-pyrazol-4-yl)pyrrolo[1,2-b]pyridazine hydrochloride (Preparation 29) and 1-(methoxymethyl)-3-oxocyclobutane-1-carbonitrile using an analogous method to that described for Example 34 and Example 35. Chiral-SFC (DAICEL CHIRALPAK OD-H, 250×30 mm, 5 μm); 60% EtOH (+0.1% NH₄OH) in CO₂).

*Peak 1, Example 42; (1s,3s)-1-(methoxymethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile as a pink solid (22.9 mg, 38.2%); LCMS m/z=432.3 [M+H]⁺; ¹H NMR (500 MHz, CDCl₃) δ: 7.75 (d, 1H), 7.69-7.71 (m, 2H), 7.56 (s, 1H), 6.51 (d, 1H), 5.71 (d, 1H), 3.94 (s, 3H), 3.68-3.72 (m, 2H), 3.59 (s, 2H), 3.44 (s, 3H), 3.28-3.30 (m, 3H), 3.15-3.18 (m, 2H), 2.58-2.59 (m, 2H), 2.12-2.14 (m, 2H), 1.89-1.96 (m, 4H).

*Peak 2, Example 43; (1r,3r)-1-(methoxymethyl)-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile as a pink solid (19.6 mg, 32.7%); LCMS m/z=432.3 [M+H]⁺; ¹H NMR (500 MHz, CDCl₃) δ: 7.76 (d, 1H), 7.69-7.71 (m, 2H), 7.56 (s, 1H), 6.51 (s, 1H), 5.72 (d, 1H), 3.94 (s, 3H), 3.68-3.71 (m, 2H), 3.55 (s, 2H), 3.46 (s, 3H), 3.21-3.30 (m, 4H), 3.05-3.10 (m, 1H), 2.39-2.46 (m, 4H), 1.88-1.93 (m, 4H).

### Example 44 and 45

### 2-((1s,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutyl)acetonitrile and 2-((1r,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutyl)acetonitrile

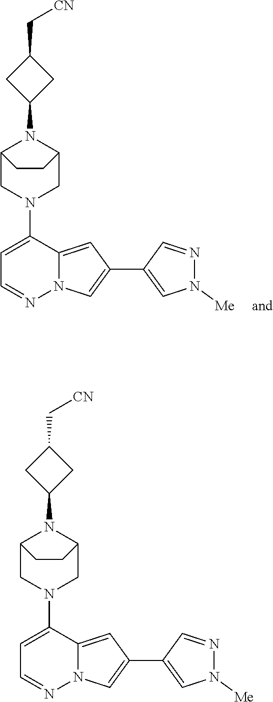 [SMILES: Cn1cc(-c2cc3c(N4CC5CCC(C4)N5[C@H]4C[C@@H](CC#N)C4)ccnn3c2)cn1 ; Cn1cc(-c2cc3c(N4CC5CCC(C4)N5[C@H]4C[C@H](CC#N)C4)ccnn3c2)cn1]

* Relative stereochemistry arbitrarily assigned

The title compounds were obtained 4-(3,8-diazabicyclo[3.2.1]octan-3-yl)-6-(1-methyl-1H-pyrazol-4-yl)pyrrolo[1,2-b]pyridazine hydrochloride (Preparation 29) and 2-(3-oxocyclobutyl)acetonitrile using an analogous method to that described for Example 34 and Example 35. SFC chromatography (SFC-3) afforded:

*Peak 1, Example 44; 2-((1s,3s)-3-(3-(6-(1-methyl-1H-pyrazol-4-yl)pyrrolo[1,2-b]pyridazin-4-yl)-3,8-diazabicyclo[3.2.1]octan-8-yl)cyclobutyl)acetonitrile as a white solid (27.9 mg); LCMS m/z=402.2 [M+H]⁺; ¹H NMR (400 MHz, CDCl₃) δ: 7.74 (d, J=5.6 Hz, 1H), 7.69-7.71 (m, 2H), 7.56 (s, 1H), 6.52 (d, J=1.6 Hz, 1H), 5.70 (d, J=1.6 Hz, 1H), 3.94 (s, 3H), 3.67-3.70 (m, 2H), 3.20-3.32 (m, 2H), 3.15-3.18 (m, 2H), 2.90-2.93 (m, 1H), 2.49-2.51 (m, 2H), 2.37-2.41 (m, 3H), 1.86-1.94 (m, 4H), 1.70-1.73 (m, 2H).

*Peak 2, Example 45; 2-((1r,3r)-3-(3-(6-(1-methyl-1H-pyrazol-4-yl)pyrrolo[1,2-b]pyridazin-4-yl)-3,8-diazabicyclo[3.2.1]octan-8-yl)cyclobutyl)acetonitrile as a white solid (6.5 mg); LCMS m/z=402.2 [M+H]⁺; ¹H NMR (400 MHz, CDCl₃) δ: 7.74-7.79 (m, 3H), 7.56 (s, 1H), 6.52 (d, J=1.6 Hz, 1H), 5.71 (d, J=5.6 Hz, 1H), 3.94 (s, 3H), 3.69-3.72 (m, 2H), 3.21-3.32 (m, 2H), 3.15-3.19 (m, 3H), 2.52 (s, 1H), 2.49-2.51 (m, 2H), 2.18-2.52 (m, 2H), 1.85-2.04 (m, 6H).

### Example 46, 47 and 48

### Rac-(1R,3R)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile and (1R,3S)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile and (1S,3R)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile

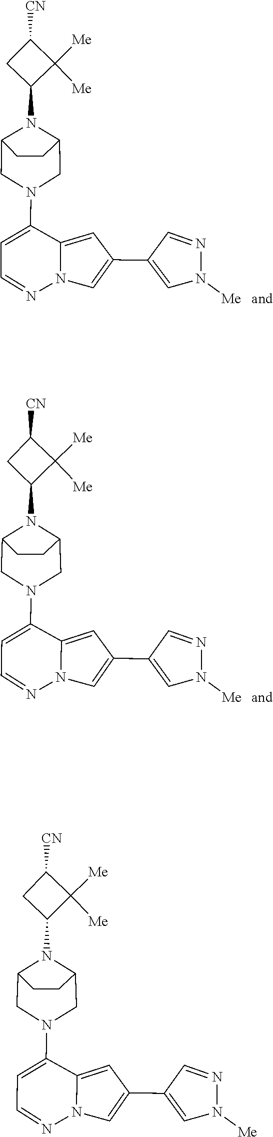 [SMILES: Cn1cc(-c2cc3c(N4CC5CCC(C4)N5[C@H]4C[C@H](C#N)C4(C)C)ccnn3c2)cn1 ; Cn1cc(-c2cc3c(N4CC5CCC(C4)N5[C@H]4C[C@@H](C#N)C4(C)C)ccnn3c2)cn1 ; Cn1cc(-c2cc3c(N4CC5CCC(C4)N5[C@@H]4C[C@H](C#N)C4(C)C)ccnn3c2)cn1]

* Structures arbitrarily assigned

The title compounds were obtained 4-(3,8-diazabicyclo[3.2.1]octan-3-yl)-6-(1-methyl-1H-pyrazol-4-yl)pyrrolo[1,2-b]pyridazine hydrochloride (Preparation 29) and 2,2-dimethyl-3-oxocyclobutane-1-carbonitrile using an analogous method to that described for Example 34 and Example 35. SFC chromatography (SFC-1, 55% EtOH).

Peak 1, Rac-(1R,3R)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile Example 46 as a pink solid (3.6, 8.4 mg); LCMS m/z=416.3 [M+H]⁺; ¹H NMR (500 MHz, CDCl₃) δ: 7.75 (d, 1H), 7.69-7.71 (m, 2H), 7.56 (s, 1H), 6.52 (s, 1H), 5.70 (d, 1H), 3.94 (s, 3H), 3.67-3.69 (m, 2H), 3.16-3.21 (m, 4H), 2.99-3.04 (m, 1H), 2.62-2.65 (m, 1H), 2.26-2.29 (m, 1H), 2.17-2.20 (m, 1H), 2.02-2.05 (m, 1H), 1.87-1.92 (m, 3H), 1.35 (s, 3H), 1.27 (s, 3H)

Peak 2, (1R,3S)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile Example 47 as a pink solid (3.6, 8.4 mg); LCMS m/z=416.3 [M+H]⁺; ¹H NMR (500 MHz, CDCl₃) δ: 7.75 (d, 1H), 7.69-7.71 (m, 2H), 7.56 (s, 1H), 6.51 (s, 1H), 5.71 (d, 1H), 3.94 (s, 3H), 3.65-3.68 (m, 2H), 3.18-3.21 (m, 4H), 2.72-2.75 (m, 1H), 2.47-2.50 (m, 1H), 2.35-2.38 (m, 1H), 2.00-2.07 (m, 2H), 1.88-2.00 (m, 3H), 1.34 (s, 3H), 1.24 (s, 3H).

Peak 3, (1S,3R)-2,2-dimethyl-3-(3-(6-(1-methyl-1H-pyrazol-4-yl)pyrrolo[1,2-b]pyridazin-4-yl)-3,8-diazabicyclo[3.2.1]octan-8-yl)cyclobutane-1-carbonitrile Example 48 as a pink solid (3.6, 8.4 mg); LCMS m/z=416.3 [M+H]⁺; ¹H NMR (500 MHz, CDCl₃) δ: 7.75 (d, 1H), 7.69-7.71 (m, 2H), 7.55 (s, 1H), 6.51 (s, 1H), 5.71 (d, 1H), 3.94 (s, 3H), 3.65-3.68 (m, 2H), 3.18-3.21 (m, 4H), 2.72-2.75 (m, 1H), 2.47-2.50 (m, 1H), 2.35-2.38 (m, 1H), 2.04-2.07 (m, 2H), 1.87-1.89 (m, 3H), 1.34 (s, 3H), 1.24 (s, 3H).

### Example 49 and 50

### Rac-(1S,2S)-2-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol and rac-(1R,2S)-2-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol

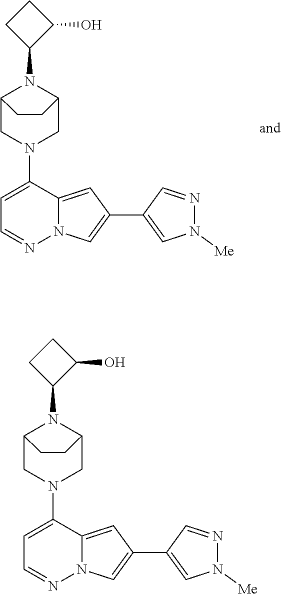 [SMILES: Cn1cc(-c2cc3c(N4CC5CCC(C4)N5[C@H]4CC[C@@H]4O)ccnn3c2)cn1 ; Cn1cc(-c2cc3c(N4CC5CCC(C4)N5[C@H]4CC[C@H]4O)ccnn3c2)cn1]

* Relative stereochemistry arbitrarily assigned

To a solution of 4-(8-(2-((tert-butyldimethylsilyl)oxy)cyclobutyl)-3,8-diazabicyclo[3.2.1]octan-3-yl)-6-(1-methyl-1H-pyrazol-4-yl)pyrrolo[1,2-b]pyridazine (Preparation 30, 428 mg, 0.869 mmol) in DCM (5 mL) was added HCl/dioxane (4 M, 5 mL) and the mixture stirred at 25° C. for 5 minutes. The mixture was evaporated to dryness in vacuo and the residue purified by prep-HPLC-3 (gradient 30-60%) to give:

*Peak 1, Example 49; rac-(1S,2S)-2-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol as a brown solid (40.6 mg, 12.5%); LCMS m/z=379.3 [M+H]⁺; ¹H NMR (400 MHz, CDCl₃) δ: 7.70-7.75 (m, 3H), 7.56 (s, 1H), 6.53 (s, 1H), 5.71 (d, 1H), 4.07-4.10 (m, 1H), 3.94 (s, 3H), 3.61-3.72 (m, 3H), 3.24-3.33 (m, 3H), 2.85-2.88 (m, 1H), 2.21-2.24 (m, 1H), 1.86-2.08 (m, 6H), 1.38-1.52 (m, 1H).

*Peak 2, Example 50; rac-(1R,2S)-2-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol as a brown solid (34.3 mg, 10.5%); LCMS m/z=379.3 [M+H]⁺; ¹H NMR (400 MHz, CDCl₃) δ: 7.70-7.78 (m, 3H), 7.57 (s, 1H), 6.52 (s, 1H), 5.73 (d, 1H), 4.13-4.16 (m, 1H), 3.94 (s, 3H), 3.68-3.79 (m, 2H), 3.13-3.39 (m, 5H), 1.85-2.06 (m, 8H).

### Example 51

### Rac-4-(8-((1S,2S)-2-methoxycyclobutyl)-3,8-diazabicyclo[3.2.1]octan-3-yl)-6-(1-methyl-1H-pyrazol-4-yl)pyrrolo[1,2-b]pyridazine

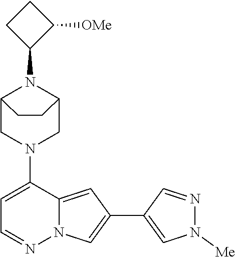 [SMILES: CO[C@H]1CC[C@@H]1N1C2CCC1CN(c1ccnn3cc(-c4cnn(C)c4)cc13)C2]

NaH (9.5 mg, 0.238 mmol, 60% purity) was added to a mixture of rac-(1S,2S)-2-(3-(6-(1-methyl-1H-pyrazol-4-yl)pyrrolo[1,2-b]pyridazin-4-yl)-3,8-diazabicyclo[3.2.1]octan-8-yl)cyclobutan-1-ol (Example 49, 30.0 mg, 0.079 mmol) in THE (2 mL) at 0° C. and stirred for 10 mins. To this was added CH₃I (33.8 mg, 0.23b mmol) and the mixture stirred at 20° C. for 18 h. The reaction mixture was concentrated and the residue purified by prep-HPLC-3 (gradient 32-62%) to afford rac-4-(8-((1S,2S)-2-methoxycyclobutyl)-3,8-diazabicyclo[3.2.1]octan-3-yl)-6-(1-methyl-1H-pyrazol-4-yl)pyrrolo[1,2-b]pyridazine as a white solid (8.1 mg, 26%). LCMS m/z=393.3 [M+H]⁺; ¹H NMR (400 MHz, CDCl₃) δ: 7.74 (d, 1H), 7.71-7.69 (m, 2H), 7.56 (s, 1H), 6.53 (s, 1H), 5.70 (d, 1H), 3.94 (s, 3H), 3.73-3.66 (m, 3H), 3.55-3.53 (m, 1H), 3.32-3.23 (m, 6H), 2.95-2.92 (m, 1H), 2.14-1.86 (m, 6H), 1.54-1.36 (m, 2H).

### Example 52

### 4-(8-(3,3-difluorocyclobutyl)-3,8-diazabicyclo[3.2.1]octan-3-yl)-6-(1-methyl-1H-pyrazol-4-yl)pyrrolo[1,2-b]pyridazine

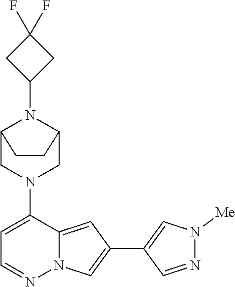 [SMILES: Cn1cc(-c2cc3c(N4CC5CCC(C4)N5C4CC(F)(F)C4)ccnn3c2)cn1]

To a solution of 6-bromo-4-(8-(3,3-difluorocyclobutyl)-3,8-diazabicyclo[3.2.1]octan-3-yl)pyrrolo[1,2-b]pyridazine (Preparation 34, 165 mg, 0.333 mmol) in dioxane (5 mL) and H₂O (0.5 mL) was added 1-methyl-4-(4,4,5,5-tetramethyl-1,3,2-dioxaborolan-2-yl)-1H-pyrazole (103.7 mg, 0.498 mmol), K₂CO₃ (91.8 mg, 0.665 mmol) and Pd(dppf)Cl₂ (24.3 mg, 0.033 mmol) and the mixture stirred at 70° C. for 1 h under N₂. The mixture was evaporated to dryness in vacuo and the residue purified by prep-HPLC-6 (gradient 44-59%) to afford 4-(8-(3,3-difluorocyclobutyl)-3,8-diazabicyclo[3.2.1]octan-3-yl)-6-(1-methyl-1H-pyrazol-4-yl)pyrrolo[1,2-b]pyridazine as a yellow solid (80 mg, 60.4%). LCMS m/z=399.1 [M+H]⁺; H NMR (400 MHz, CDCl₃) δ: 7.74 (d, 1H), 7.70-7.68 (m, 2H), 7.54 (s, 1H), 6.50 (s, 1H), 5.70 (d, 1H), 3.92 (s, 3H), 3.70-3.67 (m, 2H), 3.30-3.28 (m, 2H), 3.20-3.16 (m, 2H), 2.93-2.91 (m, 1H), 2.71-2.68 (m, 2H), 2.46-2.42 (m, 2H), 1.99-1.89 (m, 4H).

### Example 53

### 6-(6-methoxypyridazin-4-yl)-4-(8-(oxetan-3-yl)-3,8-diazabicyclo[3.2.1]octan-3-yl)pyrrolo[1,2-b]pyridazine

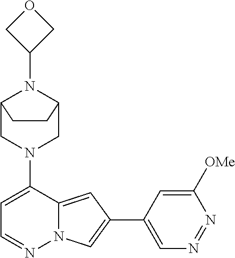 [SMILES: COc1cc(-c2cc3c(N4CC5CCC(C4)N5C4COC4)ccnn3c2)cnn1]

The title compound was prepared from 4-(3,8-diazabicyclo[3.2.1]octan-3-yl)-6-(6-methoxypyridazin-4-yl)pyrrolo[1,2-b]pyridazine hydrochloride (Preparation 40) and oxetan-3-one using an analogous method to that used for Example 9. Purification using prep-HPLC-3 (gradient 30-51%) to afford 6-(6-methoxypyridazin-4-yl)-4-(8-(oxetan-3-yl)-3,8-diazabicyclo[3.2.1]octan-3-yl)pyrrolo[1,2-b]pyridazine as a white solid (6.2 mg, 23.6%). LCMS m/z=393. [M+H]⁺; ¹H NMR (400 MHz, CDCl₃) δ: 8.05 (d, 1H), 7.95 (d, 1H), 7.82 (d, 1H), 7.07 (d, 1H), 6.72 (d, 1H), 5.77 (d, 1H), 4.73-4.77 (m, 2H), 4.57-4.61 (m, 2H), 3.82 (s, 3H), 3.69-3.73 (m, 3H), 3.26-3.34 (m, 4H), 1.89-1.98 (m, 4H).

### Example 54-57

The title compounds were prepared from the appropriate amine and ketone using an analogous method to that described for Example 9.

| Example No. | Name/Structure/HPLC/Reactants/Data |
|---|---|
| 54 | 3-(3-(6-(6-methoxypyridazin-4-yl)pyrrolo[1,2-b]pyridazin-4-y1)-3,8- |
| | diazabicyclo[3.2.1]octan-8-yl)cyclobutane-1-carbonitrile |
| | 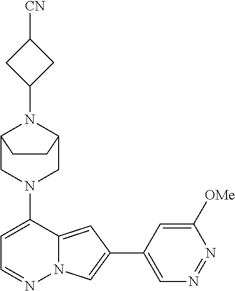 [SMILES: COc1cc(-c2cc3c(N4CC5CCC(C4)N5C4CC(C#N)C4)ccnn3c2)cnn1] |
| | Amine: 4-(3,8-diazabicyclo[3.2.1]octan-3-yl)-6-(6-methoxypyridazin-4- |
| | yl)pyrrolo[1,2-b]pyridazine hydrochloride (Preparation 40); Ketone: 3- |
| | oxocyclobutane-1-carbonitrile |
| | prep-HPLC-3 (gradient 27-57%). Yield: 7.9 mg, 28.4%; LCMS m/z = |
| | 416.2 [M + H]⁺; ¹H NMR (400 MHz, CDCl₃) δ: 8.05 (d, 1H), 7.95 (s, 1H), |
| | 7.83 (d, 1H), 7.07 (d, 1H), 6.72 (s, 1H), 5.76 (d, 1H), 3.82 (s, 3H), 3.73- |
| | 3.67 (m, 2H), 3.33-3.30 (m, 2H), 3.25-3.21 (m, 2H), 3.06-3.03 (m, 1H), |
| | 2.82-2.61 (m, 1H), 2.58-2.53 (m, 2H), 2.36-2.30 (m, 2H), 1.96-1.86 (m, |
| | 4H). |
| 55 | 3-(3-(6-(pyridazin-4-yl)pyrrolo[1,2-b]pyridazin-4-y1)-3,8- |
| | diazabicyclo[3.2.1]octan-8-yl)cyclobutane-1-carbonitrile |
| | 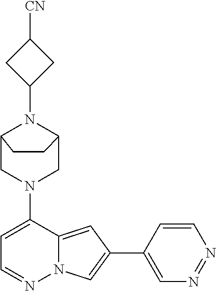 [SMILES: N#CC1CC(N2C3CCC2CN(c2ccnn4cc(-c5ccnnc5)cc24)C3)C1] |
| | Amine: 4-(3,8-diazabicyclo[3.2.1 ]octan-3-yl)-6-(pyridazin-4- |
| | yl)pyrrolo[1,2-b]pyridazine hydrochloride (Preparation 41); Ketone: 3- |
| | oxocyclobutane-1-carbonitrile |
| | prep-HPLC-3 (gradient 26-56%). Yield: 14.6 mg, 43.3%; LCMS m/z = |
| | 386.3 [M + H]⁺; ¹H NMR (400 MHz, CDCl₃) δ: 9.46 (s, 1H), 9.12 (d, 1H), |
| | 8.07 (s, 1H), 7.84 (d, 1H), 7.62-7.60 (m, 1H), 6.82 (d, 1H), 5.78 (d, 1H), |
| | 3.76-3.71 (m, 2H), 3.34-2.73 (m, 6H), 2.58-2.55 (m, 2H), 2.34-2.31 (m, |
| | 2H), 1.98-1.88 (m, 4H). |
| 56 | 4-(8-(oxetan-3-yl)-3,8-diazabicyclo[3.2.1]octan-3-yl)-6-(pyridazin-4- |
| | yl)pyrrolo[1,2-b]pyridazine |
| | 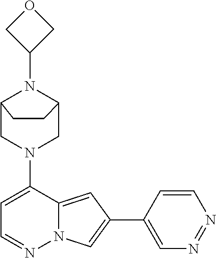 [SMILES: c1cc(-c2cc3c(N4CC5CCC(C4)N5C4COC4)ccnn3c2)cnn1] |
| | Amine: 4-(3,8-diazabicyclo[3.2.1 ]octan-3-yl)-6-(pyridazin-4- |
| | y1)pyrrolo[1,2-b]pyridazine hydrochloride (Preparation 41); Ketone: |
| | oxetan-3-one |
| | prep-HPLC-3 (gradient 17-47%). Yield: 6.8 mg, 21.4%; LCMS m/z = |
| | 363.2 [M + H]⁺; ¹H NMR (400 MHz, CDCl₃) δ: 9.45 (d, 1H), 9.10-9.12 |
| | (m, 1H), 8.06 (d, 1H), 7.82-7.85 (m, 1H), 7.60-7.63 (m,1H), 6.82-6.85 |
| | (m, 1H), 5.76-5.80 (m, 1H), 4.73-4.77 (m, 2H), 4.57-4.61 (m, 2H), 3.69- |
| | 3.77 (m, 3H), 3.27-3.37 (m, 4H), 1.88-1.98 (m, 4H). |
| 57 | 3-(3-(6-(6-methylpyridazin-4-yl)pyrrolo[1,2-b]pyridazin-4-yl)-3,8- |
| | diazabicyclo[3.2.1]octan-8-yl)cyclobutane-1-carbonitrile |
| | 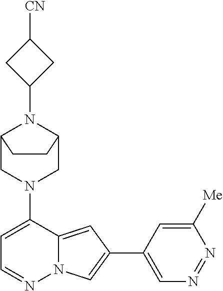 [SMILES: Cc1cc(-c2cc3c(N4CC5CCC(C4)N5C4CC(C#N)C4)ccnn3c2)cnn1] |
| | Amine: 4-(3,8-diazabicyclo[3.2.1]octan-3-yl)-6-(6-methylpyridazin-4- |
| | yl)pyrrolo[1,2-b]pyridazine hydrochloride (Preparation 42); Ketone: 3- |
| | oxocyclobutane-1-carbonitrile |
| | prep-HPLC-3 (gradient 30-60%). Yield: 2.5 mg, 8%; LCMS m/z = 400.3 |
| | [M + H]⁺; ¹H NMR (500 MHz, CDCl₃) δ: 9.29 (d, 1H), 8.05 (s, 1H), 7.83 |
| | (d, 1H), 7.46 (d, 1H), 6.81 (d, 1H), 5.78-5.75 (m, 1H), 3.76-3.70 (m, 2H), |
| | 3.34-3.24 (m, 4H), 3.06-3.03 (m, 1H), 2.82-2.80 (m, 1H), 2.75 (s, 3H), |
| | 2.58-2.55 (m, 2H), 2.35-2.31 (m, 2H), 1.95-1.89 (m, 4H). |

### Example 58 and 59

### (1R,3r)-3-(3-(6-(3-methylisoxazol-5-yl)pyrrolo[1,2-b]pyridazin-4-yl)-3,8-diazabicyclo[3.2.1]octan-8-yl)cyclobutane-1-carbonitrile and (1S,3s)-3-(3-(6-(3-methylisoxazol-5-yl)pyrrolo[1,2-b]pyridazin-4-yl)-3,8-diazabicyclo[3.2.1]octan-8-yl)cyclobutane-1-carbonitrile

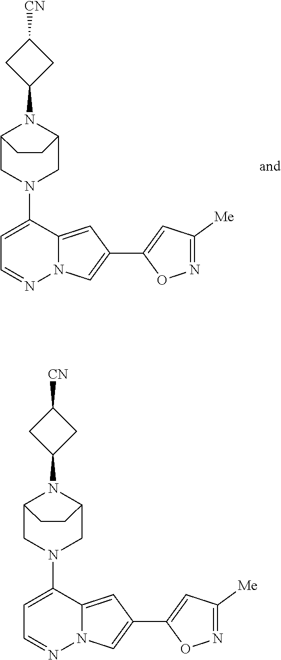 [SMILES: Cc1cc(-c2cc3c(N4CC5CCC(C4)N5[C@H]4C[C@H](C#N)C4)ccnn3c2)on1 ; Cc1cc(-c2cc3c(N4CC5CCC(C4)N5[C@H]4C[C@@H](C#N)C4)ccnn3c2)on1]

* Relative stereochemistry arbitrarily assigned

Part 1. To a solution of 3-(3-(6-bromopyrrolo[1,2-b]pyridazin-4-yl)-3,8-diazabicyclo[3.2.1]octan-8-yl)cyclobutane-1-carbonitrile (Preparation 32, 70 mg, 0.181 mmol) in dioxane (3 mL) and H₂O (0.3 mL) was added 3-methyl-5-(4,4,5,5-tetramethyl-1,3,2-dioxaborolan-2-yl)isoxazole (56.8 mg, 0.272 mmol), K₂CO₃ (50.1 mg, 0.362 mmol) and Pd(dppf)Cl₂ (13.3 mg, 0.0181 mmol) under N₂ and the mixture was stirred at 100° C. for 1 h. The mixture was concentrated and purified by prep-HPLC-3 (gradient 36-66%) to give 3-(3-(6-(3-methylisoxazol-5-yl)pyrrolo[1,2-b]pyridazin-4-yl)-3,8-diazabicyclo[3.2.1]octan-8-yl)cyclobutane-1-carbonitrile as a white solid (25 mg, 34.7%). LCMS m/z=389.3 [M+H]⁺.

Part 2. 3-(3-(6-(3-methylisoxazol-5-yl)pyrrolo[1,2-b]pyridazin-4-yl)-3,8-diazabicyclo[3.2.1]octan-8-yl)cyclobutane-1-carbonitrile (Part 1, 25 mg) was purified by SFC chromatography (SFC-7) to give:

*Peak 1, Example 58; (1R,3r)-3-(3-(6-(3-methylisoxazol-5-yl)pyrrolo[1,2-b]pyridazin-4-yl)-3,8-diazabicyclo[3.2.1]octan-8-yl)cyclobutane-1-carbonitrile (5.5 mg, 22%); LCMS m/z=389.3 [M+H]⁺; ¹H NMR (400 MHz, CDCl₃) δ: 7.94 (s, 1H), 7.80 (d, 1H), 6.80 (s, 1H), 6.23 (s, 1H), 5.73 (d, 1H), 3.71-3.75 (m, 3H), 3.31-3.36 (m, 3H), 3.15-3.23 (m, 3H), 2.48-2.50 (m, 2H), 2.34 (s, 3H), 2.30-2.32 (m, 1H), 1.88-1.98 (m, 4H).

*Peak 2, Example 59; (1S,3s)-3-(3-(6-(3-methylisoxazol-5-yl)pyrrolo[1,2-b]pyridazin-4-yl)-3,8-diazabicyclo[3.2.1]octan-8-yl)cyclobutane-1-carbonitrile (8.2 mg, 32.8%); LCMS m/z=389.3 [M+H]⁺; ¹H NMR (400 MHz, CDCl₃) δ: 7.94 (s, 1H), 7.81 (d, 1H), 6.80 (s, 1H), 6.23 (s, 1H), 5.74 (d, 1H), 3.69-3.75 (m, 3H), 3.21-3.31 (m, 4H), 3.02-3.06 (m, 1H), 2.78-2.81 (m, 1H), 2.54-2.57 (m, 2H), 2.34 (s, 3H), 2.30-2.32 (m, 1H), 1.87-1.95 (m, 4H).

### Example 60

### (1S,3s)-3-(3-(6-(1-(difluoromethyl)-1H-pyrazol-4-yl)pyrrolo[1,2-b]pyridazin-4-yl)-3,8-diazabicyclo[3.2.1]octan-8-yl)cyclobutane-1-carbonitrile

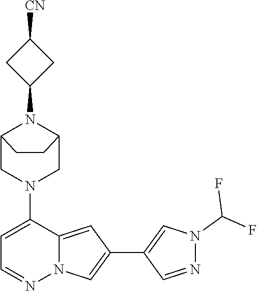 [SMILES: N#C[C@H]1C[C@@H](N2C3CCC2CN(c2ccnn4cc(-c5cnn(C(F)F)c5)cc24)C3)C1]

To a solution of (1S,3s)-3-(3-(6-bromopyrrolo[1,2-b]pyridazin-4-yl)-3,8-diazabicyclo[3.2.1]octan-8-yl)cyclobutane-1-carbonitrile (Preparation 33, 20 mg, 0.052 mmol) and 1-(difluoromethyl)-4-(4,4,5,5-tetramethyl-1,3,2-dioxaborolan-2-yl)-1H-pyrazole (25.3 mg, 0.104 mmol) in dioxane (1 mL) and H₂O (0.1 mL) was added Pd(dppf)Cl₂ (3.8 mg, 0.0052 mmol) and KF (9.0 mg, 0.155 mmol) and the mixture stirred at 70° C. for 2 h under N₂. The mixture was evaporated to dryness in vacuo and the residue purified by prep-HPLC-3 (gradient 43-73%) to give (1S,3s)-3-(3-(6-(1-(difluoromethyl)-1H-pyrazol-4-yl)pyrrolo[1,2-b]pyridazin-4-yl)-3,8-diazabicyclo[3.2.1]octan-8-yl)cyclobutane-1-carbonitrile as a white solid (12.1 mg, 55.2%). LCMS m/z=424.2 [M+H]⁺; ¹H NMR (400 MHz, CDCl₃) δ: 7.98 (s, 1H), 7.87 (s, 1H), 7.79-7.77 (m, 2H), 7.37-7.06 (m, 1H), 6.55 (s, 1H), 5.74 (d, 1H), 3.71-3.68 (m, 2H), 3.31-3.22 (m, 2H), 3.19-3.06 (m, 2H), 3.04-3.02 (m, 1H), 2.81-2.78 (m, 1H), 2.56-2.53 (m, 2H), 2.35-2.30 (m, 2H), 1.94-1.88 (m, 4H).

### Example 61 and 62

### (1S,3s)-3-(3-(6-(1-methyl-1H-pyrazol-4-yl)pyrrolo[2,1-f][1,2,4]triazin-4-yl)-3,8-diazabicyclo[3.2.1]octan-8-yl)cyclobutane-1-carbonitrile and (1R,3r)-3-(3-(6-(1-methyl-1H-pyrazol-4-yl)pyrrolo[2,1-f][1,2,4]triazin-4-yl)-3,8-diazabicyclo[3.2.1]octan-8-yl)cyclobutane-1-carbonitrile

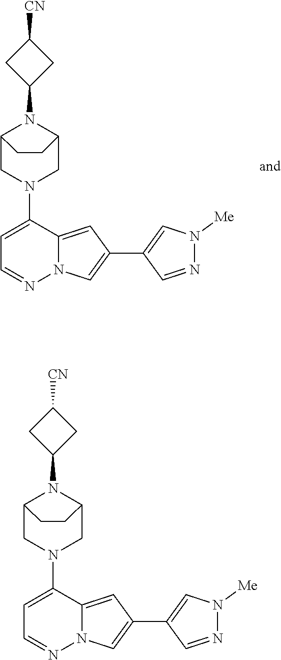 [SMILES: Cn1cc(-c2cc3c(N4CC5CCC(C4)N5[C@H]4C[C@@H](C#N)C4)ccnn3c2)cn1 ; Cn1cc(-c2cc3c(N4CC5CCC(C4)N5[C@H]4C[C@H](C#N)C4)ccnn3c2)cn1]

* Relative stereochemistry arbitrarily assigned

The title compounds were prepared from 4-(3,8-diazabicyclo[3.2.1]octan-3-yl)-6-(1-methyl-1H-pyrazol-4-yl)pyrrolo[2,1-f][1,2,4]triazine hydrochloride (Preparation 46) and 3-oxocyclobutane-1-carbonitrile using an analogous method to that described for Example 1 and 2.

Part 1. prep-HPLC-3 (gradient 23-53%) to afford 3-((1R,5S)-3-(6-(1-methyl-1H-pyrazol-4-yl)pyrrolo[2,1-f][1,2,4]triazin-4-yl)-3,8-diazabicyclo[3.2.1]octan-8-yl)cyclobutane-1-carbonitrile as a white solid (45 mg, 26.7%); LCMS m/z=389.2 [M+H]⁺.

Part 2. The compound of Part 1 was further purified by SFC-chromatography (SFC-1, 40% IPA) to give the title compounds as white solids.

*Peak 1, Example 61; (1S,3s)-3-(3-(6-(1-methyl-1H-pyrazol-4-yl)pyrrolo[2,1-f][1,2,4]triazin-4-yl)-3,8-diazabicyclo[3.2.1]octan-8-yl)cyclobutane-1-carbonitrile (22 mg, 48.9%). LCMS m/z=389.1 [M+H]⁺; ¹H NMR (400 MHz, MeOH-d₄) δ: 7.88 (s, 1H), 7.78-7.76 (m, 3H), 7.04 (s, 1H), 4.55-4.52 (m, 2H), 3.91 (s, 3H), 3.45-3.23 (m, 4H), 3.21-3.19 (m, 1H), 3.04-2.99 (m, 1H), 2.66-2.62 (m, 2H), 2.32-2.27 (m, 2H), 1.99-1.96 (m, 2H), 1.73-1.70 (m, 2H).

*Peak 2, Example 62; (1R,3r)-3-(3-(6-(1-methyl-1H-pyrazol-4-yl)pyrrolo[2,1-f][1,2,4]triazin-4-yl)-3,8-diazabicyclo[3.2.1]octan-8-yl)cyclobutane-1-carbonitrile (10.5 mg, 23.3%). LCMS m/z=389.2 [M+H]⁺; ¹H NMR (400 MHz, MeOH-d₄) δ: 7.88 (s, 1H), 7.77-7.75 (m, 3H), 7.04 (s, 1H), 4.54-4.50 (m, 2H), 3.91 (s, 3H), 3.42-3.36 (m, 5H), 3.29-3.27 (m, 1H), 2.52-2.47 (m, 2H), 2.46-2.37 (m, 2H), 1.98-1.94 (m, 2H), 1.70-1.68 (m, 2H).

### Example 63 and 64

### (1R,3r)-3-(3-(6-(1-methyl-1H-pyrazol-4-yl)-7H-pyrrolo[2,3-d]pyrimidin-4-yl)-3,8-diazabicyclo[3.2.1]octan-8-yl)cyclobutane-1-carbonitrile and (1S,3s)-3-(3-(6-(1-methyl-1H-pyrazol-4-yl)-7H-pyrrolo[2,3-d]pyrimidin-4-yl)-3,8-diazabicyclo[3.2.1]octan-8-yl)cyclobutane-1-carbonitrile

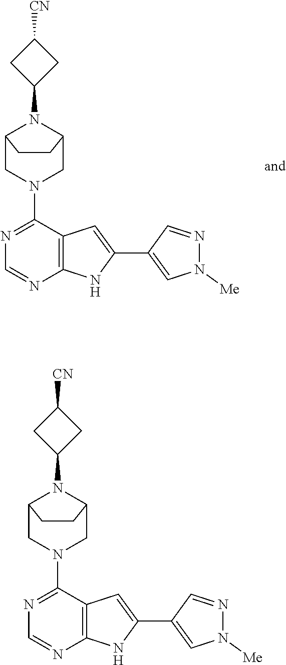 [SMILES: Cn1cc(-c2cc3c(N4CC5CCC(C4)N5[C@H]4C[C@H](C#N)C4)ncnc3[nH]2)cn1 ; Cn1cc(-c2cc3c(N4CC5CCC(C4)N5[C@H]4C[C@@H](C#N)C4)ncnc3[nH]2)cn1]

* Relative stereochemistry arbitrarily assigned

The title compounds were prepared from 4-(3,8-diazabicyclo[3.2.1]octan-3-yl)-6-(1-methyl-1H-pyrazol-4-yl)-7H-pyrrolo[2,3-d]pyrimidine hydrochloride (Preparation 52) and 3-oxocyclobutane-1-carbonitrile using an analogous method to that described for Example 1 and 2.

Part 1. prep-HPLC-3 (gradient 17-47%) to afford 3-(3-(6-(1-methyl-1H-pyrazol-4-yl)-7H-pyrrolo[2,3-d]pyrimidin-4-yl)-3,8-diazabicyclo[3.2.1]octan-8-yl)cyclobutane-1-carbonitrile as a white solid (81 mg, 48.1%). LCMS m/z=389.1 [M+H]⁺.

Part 2. The compound of Part 1 was further purified by SFC-chromatography (SFC-1, 50% EtOH) to give the title compounds as white solids.

*Peak 1, Example 63; (1R,3r)-3-(3-(6-(1-methyl-1H-pyrazol-4-yl)-7H-pyrrolo[2,3-d]pyrimidin-4-yl)-3,8-diazabicyclo[3.2.1]octan-8-yl)cyclobutane-1-carbonitrile (24.1 mg, 26.4%). LCMS m/z=389.2 [M+H]⁺; ¹H NMR (400 MHz, MeOH-d₄) δ: 8.09 (s, 1H), 7.98 (s, 1H), 7.88 (s, 1H), 6.74 (s, 1H), 4.43-4.40 (m, 2H), 3.94 (s, 3H), 3.42-3.36 (m, 6H), 2.51-2.39 (m, 4H), 1.98-1.96 (m, 2H), 1.73-1.72 (m, 2H).

*Peak 2, Example 64; (1S,3s)-3-(3-(6-(1-methyl-1H-pyrazol-4-yl)-7H-pyrrolo[2,3-d]pyrimidin-4-yl)-3,8-diazabicyclo[3.2.1]octan-8-yl)cyclobutane-1-carbonitrile (9.8 mg, 12.1%). LCMS m/z=389.2 [M+H]⁺; ¹H NMR (400 MHz, MeOH-d₄) δ: 8.10 (s, 1H), 7.98 (s, 1H), 7.88 (s, 1H), 6.74 (s, 1H), 4.46-4.42 (m, 2H), 3.94 (s, 3H), 3.48-3.40 (m, 4H), 3.27-3.25 (m, 1H), 3.03-3.01 (m, 1H), 2.66-2.64 (m, 2H), 2.35-2.30 (m, 2H), 2.00-1.97 (m, 2H), 1.78-1.75 (m, 2H).

### Example 65 and 66

### (1S,3s)-3-(3-(2-(1-methyl-1H-pyrazol-4-yl)imidazo[1,2-b]pyridazin-8-yl)-3,8-diazabicyclo[3.2.1]octan-8-yl)cyclobutane-1-carbonitrile and (1R,3r)-3-(3-(2-(1-methyl-1H-pyrazol-4-yl)imidazo[1,2-b]pyridazin-8-yl)-3,8-diazabicyclo[3.2.1]octan-8-yl)cyclobutane-1-carbonitrile

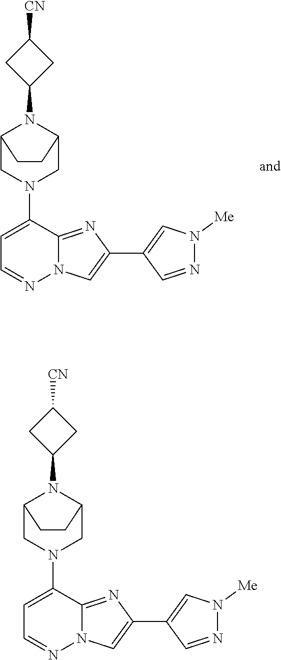 [SMILES: Cn1cc(-c2cn3nccc(N4CC5CCC(C4)N5[C@H]4C[C@@H](C#N)C4)c3n2)cn1 ; Cn1cc(-c2cn3nccc(N4CC5CCC(C4)N5[C@H]4C[C@H](C#N)C4)c3n2)cn1]

* Relative stereochemistry arbitrarily assigned

The title compounds were prepared from 8-(3,8-diazabicyclo[3.2.1]octan-3-yl)-2-(1-methyl-1H-pyrazol-4-yl)imidazo[1,2-b]pyridazine hydrochloride (Preparation 57) and 3-oxocyclobutane-1-carbonitrile using an analogous method to that described for Example 1 and 2.

Part 1, prep-HPLC-3 (gradient 28-58%) to afford 3-(3-(2-(1-methyl-1H-pyrazol-4-yl)imidazo[1,2-b]pyridazin-8-yl)-3,8-diazabicyclo[3.2.1]octan-8-yl)cyclobutane-1-carbonitrile (140 mg, 51.9%) as a white solid.

Part 2, The compound of Part 1 was further purified by SFC-chromatography (SFC-1, 45% IPA) to give the title compounds as white solids.

*Peak 1, Example 65; (1S,3s)-3-(3-(2-(1-methyl-1H-pyrazol-4-yl)imidazo[1,2-b]pyridazin-8-yl)-3,8-diazabicyclo[3.2.1]octan-8-yl)cyclobutane-1-carbonitrile Yield: 25 mg, 17.9%; LCMS m/z=389.1 [M+H]⁺; ¹H NMR (400 MHz, CDCl₃) δ: 7.87 (d, 1H), 7.82-7.81 (m, 2H), 7.75 (s, 1H), 5.88 (d, 1H), 4.65-4.62 (m, 2H), 3.94 (s, 3H), 3.35-3.32 (m, 2H), 3.30-3.22 (m, 2H), 3.07-3.02 (m, 1H), 2.82-2.77 (m, 1H), 2.57-2.50 (m, 2H), 2.36-2.33 (m, 2H), 1.89 (s, 4H).

*Peak 2, Example 66; (1R,3r)-3-(3-(2-(1-methyl-1H-pyrazol-4-yl)imidazo[1,2-b]pyridazin-8-yl)-3,8-diazabicyclo[3.2.1]octan-8-yl)cyclobutane-1-carbonitrile Yield: 65 mg, 46.3%; LCMS m/z=389.1 [M+H]⁺; ¹H NMR (400 MHz, CDCl₃) δ: 7.87 (d, 1H), 7.83-7.82 (m, 2H), 7.75 (s, 1H), 5.89 (d, 1H), 4.67-4.64 (m, 2H), 3.94 (s, 3H), 3.34-3.23 (m, 6H), 2.50-2.34 (m, 4H), 1.93-1.91 (m, 4H).

### BIOLOGICAL ASSAYS

As summarized in Table 3 below, compounds of the disclosure were assessed for their ability to inhibit TYK2, JAK1, JAK2, and JAK3 activity. The inhibitory properties of the compounds of the disclosure described herein can be evidenced by testing in any one of the following protocols.

The kinase activity of recombinantly generated catalytic kinase (also known as JH1) domain of human JAK1, JAK2, JAK3 and TYK2 were evaluated in a plate-based assay using the ADP-Glo™ Kinase Assay platform. Specifically, 4 nM of recombinant JAK1 kinase domain is used to phosphorylate 50 μM of a JAK3-342 (sequence ALVDGYFRLTT (SEQ ID NO:1)) peptide in the presence of 35 μM ATP. Catalytic activities of recombinant JAK2, JAK3 and TYK2 kinase domain (0.2, 0.3 and 2 nM, respectively) are evaluated by the phosphorylation status of the JAK3-974 (50 μM; sequence LPLDKDYYVVR (SEQ ID NO:2)) peptide with the addition of ATP (15, 4 and 10 μM, respectively). The reactions proceed for 100 minutes and the catalytic activity is quantified by first depleting the unused ATP, converting the hydrolyzed ADP into ATP to generate luminescence in a luciferase reaction; which is the basis of the ADP-Glo platform. Compounds are tested at either 10 μM or 1 μM top concentration, 11 points of 3-fold dilution. The data is normalized and the percent activity versus log concentration of compound is fitted with a 4-parameter logistic model to generate a curve and an IC50 value.

TABLE 3
Experimental Data

| | TYK2 biochem | JAK1 biochem | JAK2 biochem | JAK3 biochem |
|---|---|---|---|---|
| Ex. | IC50 (nM) | IC50 (nM) | IC50 (nM) | IC50 (nM) |
| No. | Mean | Mean | Mean | Mean |
| 1 | 13.4 | 175 | 102 | 850 |
| 2 | 1.20 | 14.7 | 9 | 68 |
| 3 | 44.0 | 430 | 236 | 1450 |
| 4 | 2.47 | 18.7 | 9.93 | 65.3 |
| 5 | 145 | 900 | 870 | 9700 |
| 6 | 305 | 1800 | 1150 | >10000 |
| 7 | 610 | >10000 | 3300 | >10000 |
| 8 | 830 | 7100 | 3150 | >10000 |
| 9 | 42.5 | 275 | 220 | >1000 |
| 10 | 226 | 340 | 180 | 1350 |
| 11 | 330 | 970 | 635 | >5000 |
| 12 | 580 | 2850 | 2300 | >10000 |
| 13 | 4.05 | 7.90 | 14 | 82 |
| 14 | 21.5 | 205 | 170 | 1800 |
| 15 | 2.13 | 13.7 | 15 | 74.3 |
| 16 | 17.5 | 315 | 117 | 550 |
| 17 | 2.80 | 36.7 | 20 | 67.7 |
| 18 | 30.5 | 49.5 | 97 | >1000 |
| 19 | 40.5 | 68 | 115 | 785 |
| 20 | 40.5 | 520 | 265 | 1400 |
| 21 | 2.87 | 60.5 | 30.5 | 150 |
| 22 | 305 | 5200 | 1200 | >10000 |
| 23 | 14.5 | 310 | 185 | 2150 |
| 24 | 19.0 | 230 | 380 | 4250 |
| 25 | 435 | 6250 | 5050 | >10000 |
| 26 | 195 | 6100 | 3300 | >10000 |
| 27 | 25.8 | 828 | 320 | 1450 |
| 28 | 2.29 | 53.6 | 20.2 | 99 |
| 29 | 187 | 6500 | 3600 | >10000 |
| 30 | 367 | >10000 | 4670 | >10000 |
| 31 | 205 | 9200 | 4000 | >10000 |
| 32 | 460 | >10000 | 6300 | >10000 |
| 33 | 42.5 | 1350 | 740 | 6200 |
| 34 | 23 | 720 | 295 | 2550 |
| 35 | 4.70 | 89 | 31.5 | 190 |
| 36 | 50.5 | 435 | 240 | 1700 |
| 37 | 310 | 4300 | 1450 | >10000 |
| 38 | 185 | 1400 | 1100 | 7300 |
| 39 | 6.95 | 88 | 54 | 460 |
| 40 | 320 | >10000 | 1900 | >10000 |
| 41 | 71 | 2200 | 420 | 8450 |
| 42 | 100 | 675 | 400 | 2400 |
| 43 | 3.70 | 73 | 39 | 260 |
| 44 | 82 | 1700 | 900 | 3550 |
| 45 | 195 | 2400 | 2700 | 9400 |
| 46 | 34 | 955 | 215 | 1850 |
| 47 | 2.33 | 72.3 | 17.3 | 130 |
| 48 | 35 | 925 | 180 | 1350 |
| 49 | 425 | >10000 | 7700 | >10000 |
| 50 | 505 | >10000 | 7350 | >10000 |
| 51 | 745 | >10000 | 6050 | >10000 |
| 52 | 1.90 | 57.2 | 35 | 574 |
| 53 | 715 | 4200 | 5700 | >10000 |
| 54 | 72 | 420 | 305 | 1600 |
| 55 | 11.5 | 130 | 80.5 | 625 |
| 56 | 90 | 820 | 950 | >10000 |
| 57 | 13 | 134 | 108 | 690 |
| 58 | 40.5 | 795 | 385 | 3450 |
| 59 | 4.25 | 65.5 | 26 | 220 |
| 60 | 2.38 | 25.5 | 17.5 | 132 |
| 61 | 104 | 1200 | 1060 | 1050 |
| 62 | 655 | >10000 | 9000 | >10000 |
| 63 | 260 | 4250 | 1800 | 1650 |
| 64 | 41.5 | 340 | 260 | 205 |
| 65 | 70.7 | 227 | 293 | 1650 |
| 66 | 3.83 | 15.1 | 21.3 | 102 |

1. A compound of Formula (I): 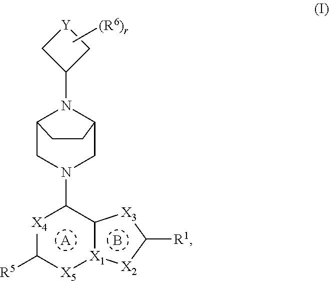
or a pharmaceutically acceptable salt thereof, wherein:
ring A and ring B together form a bicyclic heteroaryl ring;
X₁ is Nor C;
X₂ is N, NH or CR²;
X₃ is N or CR³;
X₄ is N or CR⁴;
X₅ is N or CH;
Y is CR⁷R⁸, O or NR⁹;
R¹ and R⁵ are each independently selected from H, halo, CN, —NR^{1a}R^{1b}, —OR^{1c}, C₁₋₆ alkyl, C₃₋₈ cycloalkyl, C₆₋₁₀ aryl, 4 to 10 membered heterocycloalkyl and 5 to 10 membered heteroaryl, wherein the C₁₋₆ alkyl, C₃₋₈ cycloalkyl, C₆₋₁₀ aryl, 4 to 10 membered heterocycloalkyl and 5 to 10 membered heteroaryl represented by R¹ and R⁵ are each optionally substituted with one or more R¹⁰;
R², R³, and R⁴ are each independently selected from H, halo, —CN, —NR^{1a}R^{1b}, —OR^{1c}, C₁₋₄ alkyl and C₁₋₄ haloalkyl;
R⁶, for each occurrence, is independently selected from H, halo, CN, —NR^{1a}R^{1b}, —OR^{1c}, —SO₂R^{1′} or C₁₋₆ alkyl optionally substituted with one or more substituents independently selected from halo, CN, —NR^{1a}R^{1b}, and —OR^{1c}; or two R⁶ together with the carbon atom from which they are attached form a 3 to 6 membered heterocycloalkyl or a C₃₋₆ cycloalkyl;
R⁷ and R⁸ are each independently selected from H, halo, CN, —NR^{1a}R^{1b}, —OR^{1c}, —SO₂R¹⁰ or C₁₋₆ alkyl optionally substituted with one or more substituents independently selected from halo, CN, —NR^{1a}R^{1b}, and —OR^{1c}, or R⁷ and R⁸ together with the carbon atom from which they are attached form a 3 to 6 membered heterocycloalkyl or a C₃₋₆ cycloalkyl;
R⁹ is H or C₁₋₆alkyl optionally substituted with one or more substituents independently selected from halo, CN, —NR^{1a}R^{1b}, and —OR^{1c};
R¹⁰, for each occurrence, is independently selected from halo, —CN, —NR^{1a}R^{1b}, —OR^{1c}, —C(O)OR^{1c}, C₁₋₆ alkyl, C₁₋₆ haloalkyl, C₃₋₆ cycloalkyl, C₆₋₁₀ aryl, 4 to 7 membered monocyclic heterocycloalkyl, or 5 to 6 membered heteroaryl; wherein the C₁₋₆ alkyl, C₁₋₆ haloalkyl, C₃₋₆ cycloalkyl, C₆₋₁₀ aryl, 4 to 7 membered monocyclic heterocycloalkyl, and 5 to 6 membered heteroaryl represented by R¹⁰ are each optionally substituted with one or more substituents independently selected from halo, C₁₋₄ alkyl, C₁₋₄haloalkyl, C₃₋₆ cycloalkyl, —NR^{1a}R^{1b}, —OR^{1c} and 4 to 6 membered monocyclic heterocycloalkyl;
R^{1a} and R^{1b} are each independently H or C₁₋₄ alkyl;
R^{1c} is H, C₁₋₄ alkyl or C₁₋₄ haloalkyl; and
r is 0 or an integer from 1 to 4.

2. The compound of claim 1, or a pharmaceutically acceptable salt thereof, where X₂ is NH or CR².

3. The compound of claim 1 or 2, wherein the compound is represented by Formula (II), (III), (IV), (V), (VI) or (VII): 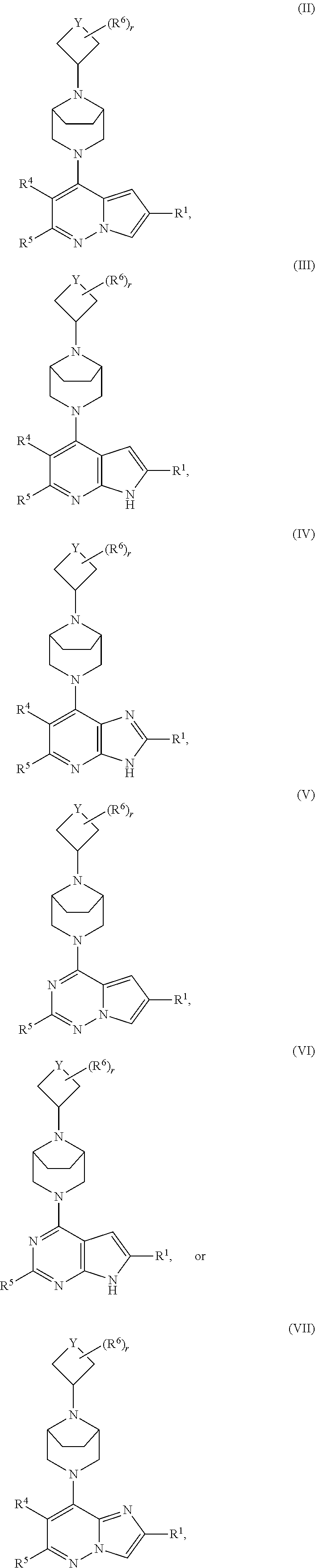
or a pharmaceutically acceptable salt thereof.

4. The compound of any one of claims 1-3, or a pharmaceutically acceptable salt thereof, wherein Y is CR⁷R⁸.

5. The compound of any one of claims 1-3, or a pharmaceutically acceptable salt thereof, wherein Y is O.

6. The compound of any one of claims 1-3, or a pharmaceutically acceptable salt thereof, wherein Y is NR⁹.

7. The compound of any one of claims 1-6, or a pharmaceutically acceptable salt thereof, wherein R¹ is a 5 to 6-membered heteroaryl optionally substituted with 1, 2 or 3 R¹⁰.

8. The compound of claim 7, or a pharmaceutically acceptable salt thereof, wherein R¹ is a 5 to 6-membered heteroaryl selected from pyrazole, imidazole, oxazole, isoxazole, thiazole, isothiazole, triazole, pyrrole, pyridine, pyridazine, pyrimidine and pyrazine, each of which is optionally substituted with 1, 2 or 3 R¹⁰.

9. The compound of claim 7, or a pharmaceutically acceptable salt thereof, wherein R¹ is a 5 to 6-membered heteroaryl selected from pyrazole, isoxazole, pyridine and pyridazine, each of which is optionally substituted with 1 or 2 R¹⁰.

10. The compound of any one of claims 1-9, or a pharmaceutically acceptable salt thereof, wherein R¹ is a 5 to 6-membered heteroaryl selected from: 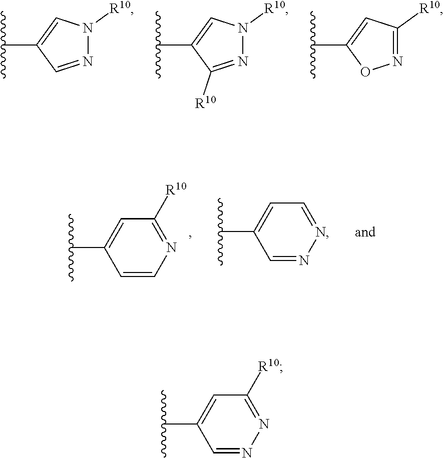
wherein 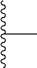
represents a point of attachment to the ring B.

11. The compound of any one of claims 1-10, or a pharmaceutically acceptable salt thereof, wherein:
R¹⁰ is independently selected from halo, —OR^{1c}, C₁₋₆ alkyl and C₁₋₆ haloalkyl; and
R^{1c} is C₁₋₄ alkyl.

12. The compound of any one of claims 1-11, or a pharmaceutically acceptable salt thereof, wherein R¹⁰ is independently selected from F, —OCH₃, —CH₃ and —CHF₂.

13. The compound of any one of claims 1-12, or a pharmaceutically acceptable salt thereof, wherein R², R³, and R⁴ are each independently selected from H, halo, C₁₋₄ alkyl and C₁₋₄ haloalkyl.

14. The compound of any one of claims 1-13, or a pharmaceutically acceptable salt thereof, wherein R², R³, and R⁴ are H.

15. The compound of any one of claims 1-14, or a pharmaceutically acceptable salt thereof, wherein R⁵ is H, halo, C₁₋₄ alkyl, C₁₋₄ haloalkyl or 5 to 6-membered heteroaryl optionally substituted with 1, 2 or 3 R¹⁰.

16. The compound of any one of claims 1-15, or a pharmaceutically acceptable salt thereof, wherein R⁵ is H.

17. The compound of any one of claims 1-16, or a pharmaceutically acceptable salt thereof, wherein:
R⁷ and R⁸ are each independently selected from H, halo, CN, —OR^{1c}, —SO₂R^{1c}, and C₁₋₆ alkyl optionally substituted with one or more substituents independently selected from halo, CN, and —OR^{1c}, or R⁷ and R⁸ together with the carbon atom from which they are attached form a 4-6 membered heterocycloalkyl; and
R^{1c} is H or C₁₋₃alkyl.

18. The compound of any one of claims 1-17, or a pharmaceutically acceptable salt thereof, wherein R⁷ and R⁸ are each independently selected from H, F, CN, —OH, —OCH₃, —SO₂CH₃, —CH₃, —CHF₂, —CF₃, —CH₂—CN, —CH₂—O—CH₃, and —CH₂CH₂—O—CH₃, or R⁷ and R⁸ together with the carbon atom from which they are attached form:

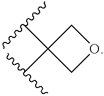 [SMILES: *C1(*)COC1]

19. The compound of any one of claims 1-18, or a pharmaceutically acceptable salt thereof, wherein r is 0, 1 or 2.

20. The compound of any one of claims 1-19, or a pharmaceutically acceptable salt thereof, wherein:
R⁶, for each occurrence, is independently selected from H, —OR^{1c}, C₁₋₄haloalkyl and C₁₋₄ alkyl optionally substituted with CN, or two R⁶ together with the carbon atoms from which they are attached form a 4-6 membered heterocycloalkyl; and
R^{1c} is H or C₁₋₃alkyl.

21. The compound of any one of claims 1-20, or a pharmaceutically acceptable salt thereof, wherein R⁶, for each occurrence, is independently selected from H, —OH, —OCH₃, —CH₂—CN, and —CH₃, or two R⁶ together with the carbon atom from which they are attached form:

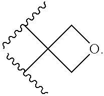 [SMILES: *C1(*)COC1]

22. The compound of claim 1-6, or a pharmaceutically acceptable salt thereof, wherein:
R¹ is a 5 to 6-membered heteroaryl optionally substituted with 1, 2 or 3 R¹⁰;
R², R³, and R⁴ are each independently selected from H, halo, C₁₋₄ alkyl and C₁₋₄ haloalkyl;
R⁵ is H, halo, C₁₋₄ alkyl, C₁₋₄ haloalkyl or 5 to 6-membered heteroaryl optionally substituted with 1, 2 or 3 R¹⁰;
R⁶, for each occurrence, is independently selected from H, —OR^{1c}, C₁₋₄haloalkyl and C₁₋₄alkyl optionally substituted with CN, or two R⁶ together with the carbon atoms from which they are attached form a 4 membered heterocycloalkyl;
R⁷ and R⁸ are each independently selected from H, halo, CN, —OR^{1c}, —SO₂R^{1c}, and C₁₋₆ alkyl optionally substituted with one or more substituents independently selected from halo, CN, and —OR^{1c}, or R⁷ and R⁸ together with the carbon atom from which they are attached form a 4 membered heterocycloalkyl;
R⁹ is H or C₁₋₆ alkyl;
R¹⁰ is independently selected from halo, —OR^{1c}, C₁₋₆ alkyl and C₁₋₆ haloalkyl;
R^{1a} and R^{1b} are each independently H or C₁₋₄ alkyl;
R^{1c} is H, C₁₋₄ alkyl; and
r is 0, 1 or 2.

23. The compound of claim 22, or a pharmaceutically acceptable salt thereof, wherein:
R¹ is a 5 to 6-membered heteroaryl selected from pyrazole, imidazole, oxazole, isoxazole, thiazole, isothiazole, triazole, pyrrole, pyridine, pyridazine, pyrimidine and pyrazine, each of which is optionally substituted with 1, 2 or 3 R¹⁰;
R², R³, and R⁴ are H;
R⁵ is H, halo, C₁₋₄ alkyl or C₁₋₄ haloalkyl;
R⁶, for each occurrence, is independently selected from H, —OR^{1c}, C₁₋₄haloalkyl and C₁₋₄ alkyl optionally substituted with CN, or two R⁶ together with the carbon atoms from which they are attached form a 4-6 membered heterocycloalkyl;
R⁷ and R⁸ are each independently selected from H, halo, CN, —OR^{1c}, —SO₂R^{1c}, and C₁₋₆ alkyl optionally substituted with one or more substituents independently selected from halo, CN, and —OR^{1c}, or R⁷ and R⁸ together with the carbon atom from which they are attached form a 4-6 membered heterocycloalkyl;
R⁹ is H or C₁₋₄alkyl;
R¹⁰ is independently selected from halo, —OR^{1c}, C₁₋₄alkyl and C₁₋₄haloalkyl; and
R^{1c} is H or C₁₋₄alkyl.

24. The compound of any one of claims 1-4 and 7-23, wherein the compound is represented by Formula (IIA), (IIB), (IIIA), (IIIB), (IVA) or (IVB): 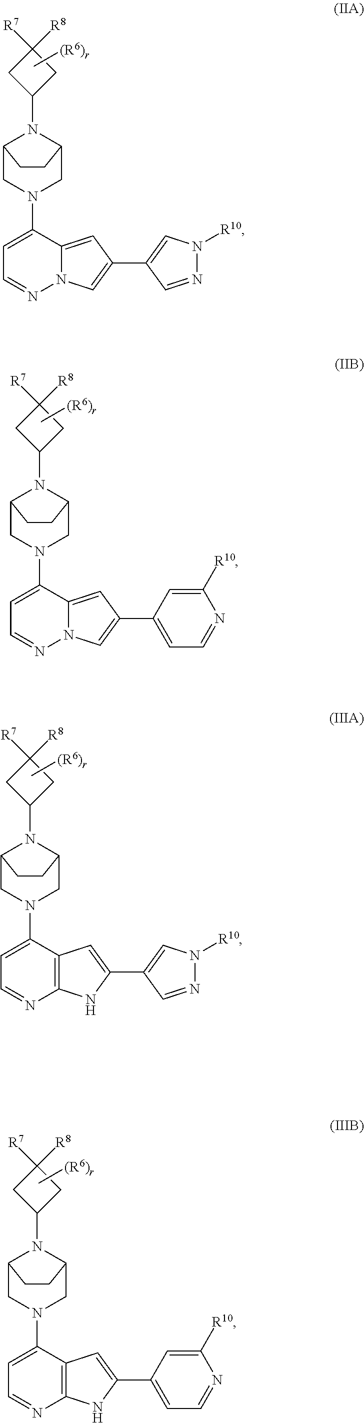 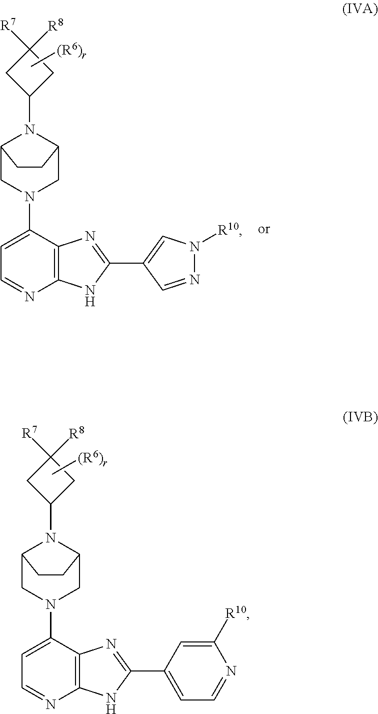
or a pharmaceutically acceptable salt thereof.

25. The compound of any one of claims 1-4 and 7-24, or a pharmaceutically acceptable salt thereof, wherein:
R⁶, for each occurrence, is independently selected from H and C₁₋₄alkyl;
R⁷ and R⁸ are each independently selected from H, halo, CN, and C₁₋₄alkyl;
R¹⁰ is selected from —OR^{1c}, C₁₋₄alkyl, and C₁₋₄haloalkyl;
R^{1c} is C₁₋₄alkyl; and
r is 0, 1 or 2.

26. The compound of any one of claims 1-4 and 7-24, or a pharmaceutically acceptable salt thereof, wherein:
R⁶ is H or CH₃;
R⁷ and R⁸ are each independently selected from H, F, CH₃ and CN;
R¹⁰ is selected from CH₃, CHF₂ and OCH₃; and
r is 0, 1 or 2.

27. The compound of any one of claims 24-26, or a pharmaceutically acceptable salt thereof, wherein r is 0.

28. A pharmaceutical composition comprising a compound according to any one of claims 1-27, or a pharmaceutically acceptable salt thereof, and a pharmaceutically acceptable carrier.

29. A method of inhibiting tyrosine kinase 2 (TYK2) activity in a subject in need thereof comprising administering to the subject an effective amount of a compound according to any one of claims 1-27 or a pharmaceutically acceptable salt thereof or a pharmaceutical composition according to claim 28.

30. A method of treating a disease or disorder responsive to inhibition of tyrosine kinase 2 (TYK2) in a subject comprising administering to the subject an effective amount of a compound according to any one of claims 1-27 or a pharmaceutically acceptable salt thereof or a pharmaceutical composition according to claim 28.

31. The method of claim 30, wherein the disease or disorder is inflammation, autoimmune disease, neuroinflammation, arthritis, rheumatoid arthritis, spondyloarthropathies, systemic lupus erythematous, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis and sunburn.
